SEQUENCE LISTING (228 sequences)

SEQ ID NO: 1 - DNA, 366 bp, synthetic construct
  misc_feature 1..366: note = Synthetic
  source 1..366: mol_type = other DNA
caggtacagctgcagcagtcaggtccaggactgctgaagccctcgcagaccctctcactcacctgtgccatctccggggacagtgtctctagcaacagtgctgcttggagttggatcaggcagtccccatcgagaggccttgagtggctgggaaggacatattacagggccaactggtttaatgattatgcactttctgtgaaaagtcgaataaccatcaacccagtcacatccacgaaccacttctccctgcagctgcactctgtgactcccgaggacacggctgtgtattactgtgcaagagaaggggctctgggatactactttgactcctggggccagggaaccctggtcaccgtctcctca

SEQ ID NO: 2 - protein, 122 aa, synthetic construct
  REGION 1..122: note = Synthetic
QVQLQQSGPGLLKPSQTLSLTCAISGDSVSSNSAAWSWIRQSPSRGLEWLGRTYYRANWFNDYALSVKSRITINPVTSTNHFSLQLHSVTPEDTAVYYCAREGALGYYFDSWGQGTLVTVSS

SEQ ID NO: 3 - DNA, 30 bp, synthetic construct
  misc_feature 1..30: note = Synthetic
  source 1..30: mol_type = other DNA
ggggacagtgtctctagcaacagtgctgct

SEQ ID NO: 4 - protein, 10 aa, synthetic construct
  REGION 1..10: note = Synthetic
GDSVSSNSAA

SEQ ID NO: 5 - DNA, 27 bp, synthetic construct
  misc_feature 1..27: note = Synthetic
  source 1..27: mol_type = other DNA
acatattacagggccaactggtttaat

SEQ ID NO: 6 - protein, 9 aa, synthetic construct
  REGION 1..9: note = Synthetic
TYYRANWFN

SEQ ID NO: 7 - DNA, 36 bp, synthetic construct
  misc_feature 1..36: note = Synthetic
  source 1..36: mol_type = other DNA
gcaagagaaggggctctgggatactactttgactcc

SEQ ID NO: 8 - protein, 12 aa, synthetic construct
  REGION 1..12: note = Synthetic
AREGALGYYFDS

SEQ ID NO: 9 - DNA, 339 bp, synthetic construct
  misc_feature 1..339: note = Synthetic
  source 1..339: mol_type = other DNA
gacatcgtgatgacccagtctccagactccctggctgtgtctctgggcgagagggccaccatcaactgcaagtccagtcaaagtgttttatacagctccaacaataagaattatttagcttggtaccaacagaaaccagggcagcctcctacactgctcatttactgggcatctacccgggaatccggggtccctgaccgattcagtggcagcgggtctgggacagatttcactctcaccatcagcagcctgcaggcggaagatgtggcaatttattactgtcaccaatattttattactccactcactttcggcggagggaccaaggtggagatcaaa

SEQ ID NO: 10 - protein, 113 aa, synthetic construct
  REGION 1..113: note = Synthetic
DIVMTQSPDSLAVSLGERATINCKSSQSVLYSSNNKNYLAWYQQKPGQPPTLLIYWASTRESGVPDRFSGSGSGTDFTLTISSLQAEDVAIYYCHQYFITPLTFGGGTKVEIK

SEQ ID NO: 11 - DNA, 36 bp, synthetic construct
  misc_feature 1..36: note = Synthetic
  source 1..36: mol_type = other DNA
caaagtgttttatacagctccaacaataagaattat

SEQ ID NO: 12 - protein, 12 aa, synthetic construct
  REGION 1..12: note = Synthetic
QSVLYSSNNKNY

SEQ ID NO: 13-14 - skipped (2 entries)

SEQ ID NO: 15 - DNA, 27 bp, synthetic construct
  misc_feature 1..27: note = Synthetic
  source 1..27: mol_type = other DNA
caccaatattttattactccactcact

SEQ ID NO: 16 - protein, 9 aa, synthetic construct
  REGION 1..9: note = Synthetic
HQYFITPLT

SEQ ID NO: 17 - DNA, 378 bp, synthetic construct
  misc_feature 1..378: note = Synthetic
  source 1..378: mol_type = other DNA
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatacaatgcagactccgtgaagggccgattcaccatctccagagacaattccaagaacacggtgtatctgcaaatgaacagcctgagagccgaggacacggctgtgtattactgtgcgagagcccggaattacgatattttgactggttattataacctcggtatggacgtctggggccaagggaccacggtcaccgtctcctca

SEQ ID NO: 18 - protein, 126 aa, synthetic construct
  REGION 1..126: note = Synthetic
QVQLVESGGGVVQPGRSLRLSCAASGFTFSSYGMHWVRQAPGKGLEWVAVIWYDGSNKYNADSVKGRFTISRDNSKNTVYLQMNSLRAEDTAVYYCARARNYDILTGYYNLGMDVWGQGTTVTVSS

SEQ ID NO: 19 - DNA, 24 bp, synthetic construct
  misc_feature 1..24: note = Synthetic
  source 1..24: mol_type = other DNA
ggattcaccttcagtagctatggc

SEQ ID NO: 20 - protein, 8 aa, synthetic construct
  REGION 1..8: note = Synthetic
GFTFSSYG

SEQ ID NO: 21 - DNA, 24 bp, synthetic construct
  misc_feature 1..24: note = Synthetic
  source 1..24: mol_type = other DNA
atatggtatgatggaagtaataaa

SEQ ID NO: 22 - protein, 8 aa, synthetic construct
  REGION 1..8: note = Synthetic
IWYDGSNK

SEQ ID NO: 23 - DNA, 57 bp, synthetic construct
  misc_feature 1..57: note = Synthetic
  source 1..57: mol_type = other DNA
gcgagagcccggaattacgatattttgactggttattataacctcggtatggacgtc

SEQ ID NO: 24 - protein, 19 aa, synthetic construct
  REGION 1..19: note = Synthetic
ARARNYDILTGYYNLGMDV

SEQ ID NO: 25 - DNA, 321 bp, synthetic construct
  misc_feature 1..321: note = Synthetic
  source 1..321: mol_type = other DNA
gacatccagatgacccagtctccatcctccctgtctgcatctgtaggagacagagtcaccatcacttgccgggcaagtcagggcattagaaatgatttaggctggtatcaacagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtctacagcataatagttacccgtacacttttggccaggggaccaagctggagatcaaa

SEQ ID NO: 26 - protein, 107 aa, synthetic construct
  REGION 1..107: note = Synthetic
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPYTFGQGTKLEIK

SEQ ID NO: 27 - DNA, 18 bp, synthetic construct
  misc_feature 1..18: note = Synthetic
  source 1..18: mol_type = other DNA
cagggcattagaaatgat

SEQ ID NO: 28 - protein, 6 aa, synthetic construct
  REGION 1..6: note = Synthetic
QGIRND

SEQ ID NO: 29-30 - skipped (2 entries)

SEQ ID NO: 31 - DNA, 27 bp, synthetic construct
  misc_feature 1..27: note = Synthetic
  source 1..27: mol_type = other DNA
ctacagcataatagttacccgtacact

SEQ ID NO: 32 - protein, 9 aa, synthetic construct
  REGION 1..9: note = Synthetic
LQHNSYPYT

SEQ ID NO: 33 - DNA, 375 bp, synthetic construct
  misc_feature 1..375: note = Synthetic
  source 1..375: mol_type = other DNA
gaagtgcagctggtggagtctgggggaaacttggtacagtctggcaggtccctgagactctcctgtacagcctctggattcgcctttgatgattttgccatgcactgggtccggcaagttccagggaagggcctggagtgggtctcaggtattagttggaatagtgataccatcggctatgcggactctgtgaagggccgattcaccatttccagagacaacgcccagaactccctgtttctgcaaatggacagtctgagagctgaggacacggccttgtattactgtgtaaaagatatggttcggggacttataggctactactactacggtatggacgtctggggccaagggaccacggtcaccgtctcctca

SEQ ID NO: 34 - protein, 125 aa, synthetic construct
  REGION 1..125: note = Synthetic
EVQLVESGGNLVQSGRSLRLSCTASGFAFDDFAMHWVRQVPGKGLEWVSGISWNSDTIGYADSVKGRFTISRDNAQNSLFLQMDSLRAEDTALYYCVKDMVRGLIGYYYYGMDVWGQGTTVTVSS

SEQ ID NO: 35 - DNA, 24 bp, synthetic construct
  misc_feature 1..24: note = Synthetic
  source 1..24: mol_type = other DNA
ggattcgcctttgatgattttgcc

SEQ ID NO: 36 - protein, 8 aa, synthetic construct
  REGION 1..8: note = Synthetic
GFAFDDFA

SEQ ID NO: 37 - DNA, 24 bp, synthetic construct
  misc_feature 1..24: note = Synthetic
  source 1..24: mol_type = other DNA
attagttggaatagtgataccatc

SEQ ID NO: 38 - protein, 8 aa, synthetic construct
  REGION 1..8: note = Synthetic
ISWNSDTI

SEQ ID NO: 39 - DNA, 54 bp, synthetic construct
  misc_feature 1..54: note = Synthetic
  source 1..54: mol_type = other DNA
gtaaaagatatggttcggggacttataggctactactactacggtatggacgtc

SEQ ID NO: 40 - protein, 18 aa, synthetic construct
  REGION 1..18: note = Synthetic
VKDMVRGLIGYYYYGMDV

SEQ ID NO: 41 - DNA, 321 bp, synthetic construct
  misc_feature 1..321: note = Synthetic
  source 1..321: mol_type = other DNA
gaaatagtgttgacgcagtctccagccatcctgtctttgtctccaggggaaagagccatcctctcctgcagggccagtcagagtatttacacctacttatcctggtaccaacagacacctggccgggctcccaggctcctcatctatgagacatccagcagggccactggcatcccagccaggttcattggcagtgggtctgggacagacttcactctcaccatcagtagcctagagcctgaagattttgcattttattactgtcagcaccgtagcgactggcctcccacttttggccaggggaccaagctggagatcaaa

SEQ ID NO: 42 - protein, 107 aa, synthetic construct
  REGION 1..107: note = Synthetic
EIVLTQSPAILSLSPGERAILSCRASQSIYTYLSWYQQTPGRAPRLLIYETSSRATGIPARFIGSGSGTDFTLTISSLEPEDFAFYYCQHRSDWPPTFGQGTKLEIK

SEQ ID NO: 43 - DNA, 18 bp, synthetic construct
  misc_feature 1..18: note = Synthetic
  source 1..18: mol_type = other DNA
cagagtatttacacctac

SEQ ID NO: 44 - protein, 6 aa, synthetic construct
  REGION 1..6: note = Synthetic
QSIYTY

SEQ ID NO: 45-46 - skipped (2 entries)

SEQ ID NO: 47 - DNA, 27 bp, synthetic construct
  misc_feature 1..27: note = Synthetic
  source 1..27: mol_type = other DNA
cagcaccgtagcgactggcctcccact

SEQ ID NO: 48 - protein, 9 aa, synthetic construct
  REGION 1..9: note = Synthetic
QHRSDWPPT

SEQ ID NO: 49 - DNA, 351 bp, synthetic construct
  misc_feature 1..351: note = Synthetic
  source 1..351: mol_type = other DNA
caggtgcagctggtggagtcggggggaggcgtggtccagcctgggaggtccctgagactctcctgtgtagcgtctggattcaccgtcagtagttatggcattcactgggtccgccaggctccaggcaagggactggagtgggtgtcagttatatggtatgatggaagaaataaagactatgcagactccgtgaagggccgattcaccatctccagagacaattccaagaacacggtgtatttggaaatgaaaggcctgagagccgaggacacggctctttattattgtgcgagagacaaaactggggattttgactcctggggccagggaaccctggtcaccgtctcctca

SEQ ID NO: 50 - protein, 117 aa, synthetic construct
  REGION 1..117: note = Synthetic
QVQLVESGGGVVQPGRSLRLSCVASGFTVSSYGIHWVRQAPGKGLEWVSVIWYDGRNKDYADSVKGRFTISRDNSKNTVYLEMKGLRAEDTALYYCARDKTGDFDSWGQGTLVTVSS

SEQ ID NO: 51 - DNA, 24 bp, synthetic construct
  misc_feature 1..24: note = Synthetic
  source 1..24: mol_type = other DNA
ggattcaccgtcagtagttatggc

SEQ ID NO: 52 - protein, 8 aa, synthetic construct
  REGION 1..8: note = Synthetic
GFTVSSYG

SEQ ID NO: 53 - DNA, 24 bp, synthetic construct
  misc_feature 1..24: note = Synthetic
  source 1..24: mol_type = other DNA
atatggtatgatggaagaaataaa

SEQ ID NO: 54 - protein, 8 aa, synthetic construct
  REGION 1..8: note = Synthetic
IWYDGRNK

SEQ ID NO: 55 - DNA, 30 bp, synthetic construct
  misc_feature 1..30: note = Synthetic
  source 1..30: mol_type = other DNA
gcgagagacaaaactggggattttgactcc

SEQ ID NO: 56 - protein, 10 aa, synthetic construct
  REGION 1..10: note = Synthetic
ARDKTGDFDS

SEQ ID NO: 57 - DNA, 321 bp, synthetic construct
  misc_feature 1..321: note = Synthetic
  source 1..321: mol_type = other DNA
gacatccagatgacccagtctccatcctccctgtctgcatctgtaggtgacagagtcaccatcacttgccgggcaagtcagaacattaacagctttttaagttggtatcagcagaaaccaggaaaagcccctaagttcctgatctatgatgcttccagtatacaaagtggggccccatcgaggttcagtggcagtggatctgggacagatttcactctcaccatcagtagtctgcaacctgaagattttgcaacttactactgtcaacagagttacagttccccgttcacttttggccaggggaccaagctggagatcaaa

SEQ ID NO: 58 - protein, 107 aa, synthetic construct
  REGION 1..107: note = Synthetic
DIQMTQSPSSLSASVGDRVTITCRASQNINSFLSWYQQKPGKAPKFLIYDASSIQSGAPSRFSGSGSGTDFTLTISSLQPEDFATYYCQQSYSSPFTFGQGTKLEIK

SEQ ID NO: 59 - DNA, 18 bp, synthetic construct
  misc_feature 1..18: note = Synthetic
  source 1..18: mol_type = other DNA
cagaacattaacagcttt

SEQ ID NO: 60 - protein, 6 aa, synthetic construct
  REGION 1..6: note = Synthetic
QNINSF

SEQ ID NO: 61-62 - skipped (2 entries)

SEQ ID NO: 63 - DNA, 27 bp, synthetic construct
  misc_feature 1..27: note = Synthetic
  source 1..27: mol_type = other DNA
caacagagttacagttccccgttcact

SEQ ID NO: 64 - protein, 9 aa, synthetic construct
  REGION 1..9: note = Synthetic
QQSYSSPFT

SEQ ID NO: 65 - DNA, 375 bp, synthetic construct
  misc_feature 1..375: note = Synthetic
  source 1..375: mol_type = other DNA
gaagtgcagctggtggagtctgggggaggcttggtacagcctggcaggtccctgagactctcctgtaaagcctctggattcgcctttgatgatttcgccatgcactgggtccggcaagctccagggaagggcctggagtgggtctcaggtattgtttggaacagtggtgacataggctatgcggactctgtgaagggccgattcaccatctccagagacaacgccaagaactccctgtatctgcaactgaatagtctgagaactgaggacacggccttgtatttctgtgtaaaagatatggttcggggacttatgggcttcaactattacggtatggacgtctggggccaagggaccacggtcaccgtctcctca

SEQ ID NO: 66 - protein, 125 aa, synthetic construct
  REGION 1..125: note = Synthetic
EVQLVESGGGLVQPGRSLRLSCKASGFAFDDFAMHWVRQAPGKGLEWVSGIVWNSGDIGYADSVKGRFTISRDNAKNSLYLQLNSLRTEDTALYFCVKDMVRGLMGFNYYGMDVWGQGTTVTVSS

SEQ ID NO: 67 - DNA, 24 bp, synthetic construct
  misc_feature 1..24: note = Synthetic
  source 1..24: mol_type = other DNA
ggattcgcctttgatgatttcgcc

SEQ ID NO: 68 - protein, 8 aa, synthetic construct
  REGION 1..8: note = Synthetic
GFAFDDFA

SEQ ID NO: 69 - DNA, 24 bp, synthetic construct
  misc_feature 1..24: note = Synthetic
  source 1..24: mol_type = other DNA
attgtttggaacagtggtgacata

SEQ ID NO: 70 - protein, 8 aa, synthetic construct
  REGION 1..8: note = Synthetic
IVWNSGDI

SEQ ID NO: 71 - DNA, 54 bp, synthetic construct
  misc_feature 1..54: note = Synthetic
  source 1..54: mol_type = other DNA
gtaaaagatatggttcggggacttatgggcttcaactattacggtatggacgtc

SEQ ID NO: 72 - protein, 18 aa, synthetic construct
  REGION 1..18: note = Synthetic
VKDMVRGLMGFNYYGMDV

SEQ ID NO: 73 - DNA, 321 bp, synthetic construct
  misc_feature 1..321: note = Synthetic
  source 1..321: mol_type = other DNA
gaaattgtgttgacgcagtctccagccaccctgtctttgtctccaggggaaagagccaccctctcctgcagggccagtcaaactattagtacttatttagtctggtaccgacagagacctggccaggctcccagtctcctcatttatgatgcatccaacagggccactgacatcccagccaggttcagtggcagtgggtctgggacagacttcactctcaccatcagcagccttgagcctgaagattttgcagtttattactgtcagcagcgtagcaactggccgatcaccttcggccaagggacacgactggagattaaa

SEQ ID NO: 74 - protein, 107 aa, synthetic construct
  REGION 1..107: note = Synthetic
EIVLTQSPATLSLSPGERATLSCRASQTISTYLVWYRQRPGQAPSLLIYDASNRATDIPARFSGSGSGTDFTLTISSLEPEDFAVYYCQQRSNWPITFGQGTRLEIK

SEQ ID NO: 75 - DNA, 18 bp, synthetic construct
  misc_feature 1..18: note = Synthetic
  source 1..18: mol_type = other DNA
caaactattagtacttat

SEQ ID NO: 76 - protein, 6 aa, synthetic construct
  REGION 1..6: note = Synthetic
QTISTY

SEQ ID NO: 77-78 - skipped (2 entries)

SEQ ID NO: 79 - DNA, 27 bp, synthetic construct
  misc_feature 1..27: note = Synthetic
  source 1..27: mol_type = other DNA
cagcagcgtagcaactggccgatcacc

SEQ ID NO: 80 - protein, 9 aa, synthetic construct
  REGION 1..9: note = Synthetic
QQRSNWPIT

SEQ ID NO: 81 - DNA, 369 bp, synthetic construct
  misc_feature 1..369: note = Synthetic
  source 1..369: mol_type = other DNA
gaagtgcagctggtggagtctgggggaggcttggtacagcctggcaggtccctgacactctcctgtgcagtctctggattcacctttgatgatcatgccatgcactgggtccggcaagctccagggaagggcctggagtgggtctcaggtattagttggaatagtgtaagtataggctatgcggactctgtgaagggccgattcacgatctccagagacaacgccaagacctccctctatctgcaaatgaacagtctgagagttgacgacacggccttatattactgtgtgcaagatttaaacgatattttgactggttatcccctctttgacttttggggccagggaaccctggtcaccgtctcctca

SEQ ID NO: 82 - protein, 123 aa, synthetic construct
  REGION 1..123: note = Synthetic
EVQLVESGGGLVQPGRSLTLSCAVSGFTFDDHAMHWVRQAPGKGLEWVSGISWNSVSIGYADSVKGRFTISRDNAKTSLYLQMNSLRVDDTALYYCVQDLNDILTGYPLFDFWGQGTLVTVSS

SEQ ID NO: 83 - DNA, 24 bp, synthetic construct
  misc_feature 1..24: note = Synthetic
  source 1..24: mol_type = other DNA
ggattcacctttgatgatcatgcc

SEQ ID NO: 84 - protein, 8 aa, synthetic construct
  REGION 1..8: note = Synthetic
GFTFDDHA

SEQ ID NO: 85 - DNA, 24 bp, synthetic construct
  misc_feature 1..24: note = Synthetic
  source 1..24: mol_type = other DNA
attagttggaatagtgtaagtata

SEQ ID NO: 86 - protein, 8 aa, synthetic construct
  REGION 1..8: note = Synthetic
ISWNSVSI

SEQ ID NO: 87 - DNA, 48 bp, synthetic construct
  misc_feature 1..48: note = Synthetic
  source 1..48: mol_type = other DNA
gtgcaagatttaaacgatattttgactggttatcccctctttgacttt

SEQ ID NO: 88 - protein, 16 aa, synthetic construct
  REGION 1..16: note = Synthetic
VQDLNDILTGYPLFDF

SEQ ID NO: 89 - DNA, 324 bp, synthetic construct
  misc_feature 1..324: note = Synthetic
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 90 - protein, 108 aa, synthetic construct
  REGION 1..108: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 91 - DNA, 18 bp, synthetic construct
  misc_feature 1..18: note = Synthetic
  source 1..18: mol_type = other DNA
cagagcattagcagctat

SEQ ID NO: 92 - protein, 6 aa, synthetic construct
  REGION 1..6: note = Synthetic
QSISSY

SEQ ID NO: 93-94 - skipped (2 entries)

SEQ ID NO: 95 - DNA, 30 bp, synthetic construct
  misc_feature 1..30: note = Synthetic
  source 1..30: mol_type = other DNA
caacagagttacagtacccctccgatcacc

SEQ ID NO: 96 - protein, 10 aa, synthetic construct
  REGION 1..10: note = Synthetic
QQSYSTPPIT

SEQ ID NO: 97 - DNA, 369 bp, synthetic construct
  misc_feature 1..369: note = Synthetic
  source 1..369: mol_type = other DNA
gaagtgcagctggtggagtctgggggaggcttggtacaggctggcaggtccctaagactctcctgtgaagcctctggattcacctttgatgattatggcatgcactgggtccggcaaggtccagggaagggcctggagtgggtctcaggtattagttggaatagtggtaacatagactatgcggactctgtgaagggccgattcaccatctccagagacaacgccaagacctccctgtatctgcaaatgaacagtctgaaaactgacgacacggccttgtatttctgtgcaaaagatgctgtggggtttaactggaactactttctctttgactactggggccagggaaccctggtcaccgtctcctca

SEQ ID NO: 98 - protein, 123 aa, synthetic construct
  REGION 1..123: note = Synthetic
EVQLVESGGGLVQAGRSLRLSCEASGFTFDDYGMHWVRQGPGKGLEWVSGISWNSGNIDYADSVKGRFTISRDNAKTSLYLQMNSLKTDDTALYFCAKDAVGFNWNYFLFDYWGQGTLVTVSS

SEQ ID NO: 99 - DNA, 24 bp, synthetic construct
  misc_feature 1..24: note = Synthetic
  source 1..24: mol_type = other DNA
ggattcacctttgatgattatggc

SEQ ID NO: 100 - protein, 8 aa, synthetic construct
  REGION 1..8: note = Synthetic
GFTFDDYG

SEQ ID NO: 101 - DNA, 24 bp, synthetic construct
  misc_feature 1..24: note = Synthetic
  source 1..24: mol_type = other DNA
attagttggaatagtggtaacata

SEQ ID NO: 102 - protein, 8 aa, synthetic construct
  REGION 1..8: note = Synthetic
ISWNSGNI

SEQ ID NO: 103 - DNA, 48 bp, synthetic construct
  misc_feature 1..48: note = Synthetic
  source 1..48: mol_type = other DNA
gcaaaagatgctgtggggtttaactggaactactttctctttgactac

SEQ ID NO: 104 - protein, 16 aa, synthetic construct
  REGION 1..16: note = Synthetic
AKDAVGFNWNYFLFDY

SEQ ID NO: 105 - DNA, 351 bp, synthetic construct
  misc_feature 1..351: note = Synthetic
  source 1..351: mol_type = other DNA
caggtgcagctggtggagtctgggggaggcgtggtccagcctgggaggtccctgagactcacctgtgtagcgtctggattcaccgtcagtagttatggaatgcactgggtccgccaggccccaggcaaggggctggagtgggtggcagttatgttttatgatgaaagtaaaaaatattatgcagactccgtgaagggccgattcaccatctccagagacaattccaagaacacgctgtatctgcaaatgaacagcctgagagtcgaggacacggctgtgtattactgtgcgagagatgaacagctcgactttgaatactggggccagggaaccctggtcaccgtctcctca

SEQ ID NO: 106 - protein, 117 aa, synthetic construct
  REGION 1..117: note = Synthetic
QVQLVESGGGVVQPGRSLRLTCVASGFTVSSYGMHWVRQAPGKGLEWVAVMFYDESKKYYADSVKGRFTISRDNSKNTLYLQMNSLRVEDTAVYYCARDEQLDFEYWGQGTLVTVSS

SEQ ID NO: 107 - DNA, 24 bp, synthetic construct
  misc_feature 1..24: note = Synthetic
  source 1..24: mol_type = other DNA
ggattcaccgtcagtagttatgga

SEQ ID NO: 108 - protein, 8 aa, synthetic construct
  REGION 1..8: note = Synthetic
GFTVSSYG

SEQ ID NO: 109 - DNA, 24 bp, synthetic construct
  misc_feature 1..24: note = Synthetic
  source 1..24: mol_type = other DNA
atgttttatgatgaaagtaaaaaa

SEQ ID NO: 110 - protein, 8 aa, synthetic construct
  REGION 1..8: note = Synthetic
MFYDESKK

SEQ ID NO: 111 - DNA, 30 bp, synthetic construct
  misc_feature 1..30: note = Synthetic
  source 1..30: mol_type = other DNA
gcgagagatgaacagctcgactttgaatac

SEQ ID NO: 112 - protein, 10 aa, synthetic construct
  REGION 1..10: note = Synthetic
ARDEQLDFEY

SEQ ID NO: 113 - DNA, 369 bp, synthetic construct
  misc_feature 1..369: note = Synthetic
  source 1..369: mol_type = other DNA
gaagtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaagatataatggggaactgggactacttctacggtatggacgtctggggccaagggaccacggtcaccgtctcctca

SEQ ID NO: 114 - protein, 123 aa, synthetic construct
  REGION 1..123: note = Synthetic
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDIMGNWDYFYGMDVWGQGTTVTVSS

SEQ ID NO: 115 - DNA, 24 bp, synthetic construct
  misc_feature 1..24: note = Synthetic
  source 1..24: mol_type = other DNA
ggattcacctttgatgattatgcc

SEQ ID NO: 116 - protein, 8 aa, synthetic construct
  REGION 1..8: note = Synthetic
GFTFDDYA

SEQ ID NO: 117 - DNA, 24 bp, synthetic construct
  misc_feature 1..24: note = Synthetic
  source 1..24: mol_type = other DNA
attagttggaatagtggtagcata

SEQ ID NO: 118 - protein, 8 aa, synthetic construct
  REGION 1..8: note = Synthetic
ISWNSGSI

SEQ ID NO: 119 - DNA, 48 bp, synthetic construct
  misc_feature 1..48: note = Synthetic
  source 1..48: mol_type = other DNA
gcaaaagatataatggggaactgggactacttctacggtatggacgtc

SEQ ID NO: 120 - protein, 16 aa, synthetic construct
  REGION 1..16: note = Synthetic
AKDIMGNWDYFYGMDV

SEQ ID NO: 121 - DNA, 369 bp, synthetic construct
  misc_feature 1..369: note = Synthetic
  source 1..369: mol_type = other DNA
gaagtgcagctggtggagtctgggggaggcttggtacagcctggcaggtccctgagactctcctgtgcagcctctggattcacctttgatgataatgccatgcactgggtccggcaacctccagggaagggcctggagtgggtctcaggtattagttggaatagtggaagcataggctatgcggactctgtgaagggccgattcaccatctccagagacaacgccaagaactccctgtatctgcaaatgaacagtctgagagctgaggacacggccttgtattactgtgcaaaagatataaacgatattttgactggttatcctctttttgattactggggccagggaaccctggtcaccgtctcctca

SEQ ID NO: 122 - protein, 123 aa, synthetic construct
  REGION 1..123: note = Synthetic
EVQLVESGGGLVQPGRSLRLSCAASGFTFDDNAMHWVRQPPGKGLEWVSGISWNSGSIGYADSVKGRFTISRDNAKNSLYLQMNSLRAEDTALYYCAKDINDILTGYPLFDYWGQGTLVTVSS

SEQ ID NO: 123 - DNA, 24 bp, synthetic construct
  misc_feature 1..24: note = Synthetic
  source 1..24: mol_type = other DNA
ggattcacctttgatgataatgcc

SEQ ID NO: 124 - protein, 8 aa, synthetic construct
  REGION 1..8: note = Synthetic
GFTFDDNA

SEQ ID NO: 125 - DNA, 24 bp, synthetic construct
  misc_feature 1..24: note = Synthetic
  source 1..24: mol_type = other DNA
attagttggaatagtggaagcata

SEQ ID NO: 126 - protein, 8 aa, synthetic construct
  REGION 1..8: note = Synthetic
ISWNSGSI

SEQ ID NO: 127 - DNA, 48 bp, synthetic construct
  misc_feature 1..48: note = Synthetic
  source 1..48: mol_type = other DNA
gcaaaagatataaacgatattttgactggttatcctctttttgattac

SEQ ID NO: 128 - protein, 16 aa, synthetic construct
  REGION 1..16: note = Synthetic
AKDINDILTGYPLFDY

SEQ ID NO: 129 - DNA, 378 bp, synthetic construct
  misc_feature 1..378: note = Synthetic
  source 1..378: mol_type = other DNA
gaagtgcagctggtggagtctgggggaggcgtggtacagcctggggggtccctgagactctcctgtgcagcctctacattcacctttgatgattttgccatgcactgggtccgtcaagctccagggaagggtctggagtgggtctctcttattactggggatggtggtagcacatactatgcagaccctgtgaagggccgattcaccatctccagagacaacagcaaaaactccctgtatctgcaaatgaacagtctgagaactgaggacaccgccttgtattactgtgtaaaagattggtggatagcagctcgtccggactactactactacggtatggacgtctggggccaagggaccacggtcaccgtctcctca

SEQ ID NO: 130 - protein, 126 aa, synthetic construct
  REGION 1..126: note = Synthetic
EVQLVESGGGVVQPGGSLRLSCAASTFTFDDFAMHWVRQAPGKGLEWVSLITGDGGSTYYADPVKGRFTISRDNSKNSLYLQMNSLRTEDTALYYCVKDWWIAARPDYYYYGMDVWGQGTTVTVSS

SEQ ID NO: 131 - DNA, 24 bp, synthetic construct
  misc_feature 1..24: note = Synthetic
  source 1..24: mol_type = other DNA
acattcacctttgatgattttgcc

SEQ ID NO: 132 - protein, 8 aa, synthetic construct
  REGION 1..8: note = Synthetic
TFTFDDFA

SEQ ID NO: 133 - DNA, 24 bp, synthetic construct
  misc_feature 1..24: note = Synthetic
  source 1..24: mol_type = other DNA
attactggggatggtggtagcaca

SEQ ID NO: 134 - protein, 8 aa, synthetic construct
  REGION 1..8: note = Synthetic
ITGDGGST

SEQ ID NO: 135 - DNA, 57 bp, synthetic construct
  misc_feature 1..57: note = Synthetic
  source 1..57: mol_type = other DNA
gtaaaagattggtggatagcagctcgtccggactactactactacggtatggacgtc

SEQ ID NO: 136 - protein, 19 aa, synthetic construct
  REGION 1..19: note = Synthetic
VKDWWIAARPDYYYYGMDV

SEQ ID NO: 137 - DNA, 378 bp, synthetic construct
  misc_feature 1..378: note = Synthetic
  source 1..378: mol_type = other DNA
caggtgcagctgcaggagtcgggcccaggactggtgaagccctcggagaccctgtccatcacctgcactgtctctggtggctccttcagtagtcacttctggacctggatccggcagcccccaggaaagggactggaatggattggatatctccattatagtgggggcaccagctacaacccctccctcaagagtcgagtcatcatatcagtggacacgtccaagaaccagttctccctgaaactgaactctgtgaccgctgcggacacggccgtgtattactgtgcgagagctagatcggggattacttttgggggacttatcgtccctggttcttttgatatctggggccaagggacaatggtcaccgtctcttca

SEQ ID NO: 138 - protein, 126 aa, synthetic construct
  REGION 1..126: note = Synthetic
QVQLQESGPGLVKPSETLSITCTVSGGSFSSHFWTWIRQPPGKGLEWIGYLHYSGGTSYNPSLKSRVIISVDTSKNQFSLKLNSVTAADTAVYYCARARSGITFGGLIVPGSFDIWGQGTMVTVSS

SEQ ID NO: 139 - DNA, 24 bp, synthetic construct
  misc_feature 1..24: note = Synthetic
  source 1..24: mol_type = other DNA
ggtggctccttcagtagtcacttc

SEQ ID NO: 140 - protein, 8 aa, synthetic construct
  REGION 1..8: note = Synthetic
GGSFSSHF

SEQ ID NO: 141 - DNA, 21 bp, synthetic construct
  misc_feature 1..21: note = Synthetic
  source 1..21: mol_type = other DNA
ctccattatagtgggggcacc

SEQ ID NO: 142 - protein, 7 aa, synthetic construct
  REGION 1..7: note = Synthetic
LHYSGGT

SEQ ID NO: 143 - DNA, 60 bp, synthetic construct
  misc_feature 1..60: note = Synthetic
  source 1..60: mol_type = other DNA
gcgagagctagatcggggattacttttgggggacttatcgtccctggttcttttgatatc

SEQ ID NO: 144 - protein, 20 aa, synthetic construct
  REGION 1..20: note = Synthetic
ARARSGITFGGLIVPGSFDI

SEQ ID NO: 145 - DNA, 324 bp, synthetic construct
  misc_feature 1..324: note = Synthetic
  source 1..324: mol_type = other DNA
gaaatagttttgacacagagtcccggcacactgtcactctctcccggggaaagagccaccttgtcatgtagagcaagtcagtcagtctctagctcttatctcgcctggtaccagcagaagccgggacaggcccctagactgctgatctacggggcaagttccagggccaccggaatccccgaccggttcagtggaagcggaagcggaaccgattttactttgacgatttctagactggagccagaggatttcgccgtttactattgtcaacagtacggaagcagcccgtggacgtttggccagggcacgaaggtagaaatcaag

SEQ ID NO: 146 - protein, 108 aa, synthetic construct
  REGION 1..108: note = Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 147 - DNA, 21 bp, synthetic construct
  misc_feature 1..21: note = Synthetic
  source 1..21: mol_type = other DNA
cagtcagtctctagctcttat

SEQ ID NO: 148 - protein, 7 aa, synthetic construct
  REGION 1..7: note = Synthetic
QSVSSSY

SEQ ID NO: 149-150 - skipped (2 entries)

SEQ ID NO: 151 - DNA, 27 bp, synthetic construct
  misc_feature 1..27: note = Synthetic
  source 1..27: mol_type = other DNA
caacagtacggaagcagcccgtggacg

SEQ ID NO: 152 - protein, 9 aa, synthetic construct
  REGION 1..9: note = Synthetic
QQYGSSPWT

SEQ ID NO: 153 - DNA, 378 bp, synthetic construct
  misc_feature 1..378: note = Synthetic
  source 1..378: mol_type = other DNA
caggtgcagctgcaggagtcgggcccaggactggtgaagccttcggagaccctgtccctcatttgttctgtctctggtggctccttcagtagtcacttctggagttggatccggcagcccccagggaagggactggagtggattgggtatgtcctttacagtgggggcaccaattacaacccctccctcaagagtcgagtcaccatatcagtagacacgtccaagaatcagttcttcctgaaactgagctctgtgaccgctgcggacacggccgattattactgtgcgagagctatatcggggattacgtttgggggaattatcgtccctggttcttttgatatctggggccaagggacaatggtcaccgtctcttca

SEQ ID NO: 154 - protein, 126 aa, synthetic construct
  REGION 1..126: note = Synthetic
QVQLQESGPGLVKPSETLSLICSVSGGSFSSHFWSWIRQPPGKGLEWIGYVLYSGGTNYNPSLKSRVTISVDTSKNQFFLKLSSVTAADTADYYCARAISGITFGGIIVPGSFDIWGQGTMVTVSS

SEQ ID NO: 155 - DNA, 24 bp, synthetic construct
  misc_feature 1..24: note = Synthetic
  source 1..24: mol_type = other DNA
ggtggctccttcagtagtcacttc

SEQ ID NO: 156 - protein, 8 aa, synthetic construct
  REGION 1..8: note = Synthetic
GGSFSSHF

SEQ ID NO: 157 - DNA, 21 bp, synthetic construct
  misc_feature 1..21: note = Synthetic
  source 1..21: mol_type = other DNA
gtcctttacagtgggggcacc

SEQ ID NO: 158 - protein, 7 aa, synthetic construct
  REGION 1..7: note = Synthetic
VLYSGGT

SEQ ID NO: 159 - DNA, 60 bp, synthetic construct
  misc_feature 1..60: note = Synthetic
  source 1..60: mol_type = other DNA
gcgagagctatatcggggattacgtttgggggaattatcgtccctggttcttttgatatc

SEQ ID NO: 160 - protein, 20 aa, synthetic construct
  REGION 1..20: note = Synthetic
ARAISGITFGGIIVPGSFDI

SEQ ID NO: 161 - DNA, 378 bp, synthetic construct
  misc_feature 1..378: note = Synthetic
  source 1..378: mol_type = other DNA
caggtgcagctgcaggagtcgggcccaggactggtgaagccctcggagaccctgtccctcacctgcactgtctctggtggctccttcagtagtcacttctggagctggatccggcagcccccagggaagggactggagtggattggatatatcttatacactgggggcaccagcttcaacccctccctcaagagtcgagtctccatgtcagtgggcacgtccaagaaccagttctccctgaaattgagctctgtgaccgctgcggacacggccgtatattactgtgcgagagctagatcggggataacgtttacgggtattatcgtccctggctcttttgatatctggggccaagggacaatggtcaccgtctcttca

SEQ ID NO: 162 - protein, 126 aa, synthetic construct
  REGION 1..126: note = Synthetic
QVQLQESGPGLVKPSETLSLTCTVSGGSFSSHFWSWIRQPPGKGLEWIGYILYTGGTSFNPSLKSRVSMSVGTSKNQFSLKLSSVTAADTAVYYCARARSGITFTGIIVPGSFDIWGQGTMVTVSS

SEQ ID NO: 163 - DNA, 24 bp, synthetic construct
  misc_feature 1..24: note = Synthetic
  source 1..24: mol_type = other DNA
ggtggctccttcagtagtcacttc

SEQ ID NO: 164 - protein, 8 aa, synthetic construct
  REGION 1..8: note = Synthetic
GGSFSSHF

SEQ ID NO: 165 - DNA, 21 bp, synthetic construct
  misc_feature 1..21: note = Synthetic
  source 1..21: mol_type = other DNA
atcttatacactgggggcacc

SEQ ID NO: 166 - protein, 7 aa, synthetic construct
  REGION 1..7: note = Synthetic
ILYTGGT

SEQ ID NO: 167 - DNA, 60 bp, synthetic construct
  misc_feature 1..60: note = Synthetic
  source 1..60: mol_type = other DNA
gcgagagctagatcggggataacgtttacgggtattatcgtccctggctcttttgatatc

SEQ ID NO: 168 - protein, 20 aa, synthetic construct
  REGION 1..20: note = Synthetic
ARARSGITFTGIIVPGSFDI

SEQ ID NO: 169 - DNA, 378 bp, synthetic construct
  misc_feature 1..378: note = Synthetic
  source 1..378: mol_type = other DNA
caggtgcagctgcaggagtcgggcccaggactggtgaagccttcggagaccctgtccctcacttgttctgtctctggtggctccttcagtagtcacttctggagctggatccggcagcccccagggaagggactggagtggattggatatatccattacagtgggggcaccaattacaacccctccctcaagagtcgagtcaccatatcagtagacacgtccaagaaccagttctcccttaaactgacttctgtgaccgctgcggacacggccgattattactgtgcgagagctatatcggggattacgtttgggggaatgatcgtccctggttcttttgatgtctggggcgaagggacaatggtcaccgtctcttca

SEQ ID NO: 170 - protein, 126 aa, synthetic construct
  REGION 1..126: note = Synthetic
QVQLQESGPGLVKPSETLSLTCSVSGGSFSSHFWSWIRQPPGKGLEWIGYIHYSGGTNYNPSLKSRVTISVDTSKNQFSLKLTSVTAADTADYYCARAISGITFGGMIVPGSFDVWGEGTMVTVSS

SEQ ID NO: 171 - DNA, 24 bp, synthetic construct
  misc_feature 1..24: note = Synthetic
  source 1..24: mol_type = other DNA
ggtggctccttcagtagtcacttc

SEQ ID NO: 172 - protein, 8 aa, synthetic construct
  REGION 1..8: note = Synthetic
GGSFSSHF

SEQ ID NO: 173 - DNA, 21 bp, synthetic construct
  misc_feature 1..21: note = Synthetic
  source 1..21: mol_type = other DNA
atccattacagtgggggcacc

SEQ ID NO: 174 - protein, 7 aa, synthetic construct
  REGION 1..7: note = Synthetic
IHYSGGT

SEQ ID NO: 175 - DNA, 60 bp, synthetic construct
  misc_feature 1..60: note = Synthetic
  source 1..60: mol_type = other DNA
gcgagagctatatcggggattacgtttgggggaatgatcgtccctggttcttttgatgtc

SEQ ID NO: 176 - protein, 20 aa, synthetic construct
  REGION 1..20: note = Synthetic
ARAISGITFGGMIVPGSFDV

SEQ ID NO: 177 - DNA, 378 bp, synthetic construct
  misc_feature 1..378: note = Synthetic
  source 1..378: mol_type = other DNA
caggtgcagctgcaggagtcgggcccaggactggtgaagccttcggagaccctgtccctcacctgcactgtctctggtggcaccttcagtagtcacttctggagctggatccggcagcccccaggaaagggactggagtggattggatatatcttttacactgggggcaccaaccacaacccctccctcaagagtcgagtcaccatatcaatagacacgtccaagaaccagttctccctgaaactgacctctgtgaccgctgcggacacggccgtgtattactgtgcgagagctagatcggggattacgtttgggggagttatcgtccctggttcttttgatatctggggccaagggacaatggtcaccgtctcttca

SEQ ID NO: 178 - protein, 126 aa, synthetic construct
  REGION 1..126: note = Synthetic
QVQLQESGPGLVKPSETLSLTCTVSGGTFSSHFWSWIRQPPGKGLEWIGYIFYTGGTNHNPSLKSRVTISIDTSKNQFSLKLTSVTAADTAVYYCARARSGITFGGVIVPGSFDIWGQGTMVTVSS

SEQ ID NO: 179 - DNA, 24 bp, synthetic construct
  misc_feature 1..24: note = Synthetic
  source 1..24: mol_type = other DNA
ggtggcaccttcagtagtcacttc

SEQ ID NO: 180 - protein, 8 aa, synthetic construct
  REGION 1..8: note = Synthetic
GGTFSSHF

SEQ ID NO: 181 - DNA, 21 bp, synthetic construct
  misc_feature 1..21: note = Synthetic
  source 1..21: mol_type = other DNA
atcttttacactgggggcacc

SEQ ID NO: 182 - protein, 7 aa, synthetic construct
  REGION 1..7: note = Synthetic
IFYTGGT

SEQ ID NO: 183 - DNA, 60 bp, synthetic construct
  misc_feature 1..60: note = Synthetic
  source 1..60: mol_type = other DNA
gcgagagctagatcggggattacgtttgggggagttatcgtccctggttcttttgatatc

SEQ ID NO: 184 - protein, 20 aa, synthetic construct
  REGION 1..20: note = Synthetic
ARARSGITFGGVIVPGSFDI

SEQ ID NO: 185 - DNA, 378 bp, synthetic construct
  misc_feature 1..378: note = Synthetic
  source 1..378: mol_type = other DNA
caggtgcagctgcaggagtcgggcccaggactggtgaaaccttcggagaccctgtccctcacctgcactgtctctggtggctccttcagcagtcacttctggaactggatccggcagtccccagggaggggactggaatggattggatatatctattacagtgggggcaccaactataacccctccttcaagagtcgagtcaccatgtcagtagacacgtccaagaaccagttctccctgaaactgagctctgtgaccgctgcggacacggccgtgttttactgtgcgagagctagatcggggataacgtttgggggagttctcgtccctggttcttttgatatttggggccaagggacaatggtcaccgtctcttca

SEQ ID NO: 186 - protein, 126 aa, synthetic construct
  REGION 1..126: note = Synthetic
QVQLQESGPGLVKPSETLSLTCTVSGGSFSSHFWNWIRQSPGRGLEWIGYIYYSGGTNYNPSFKSRVTMSVDTSKNQFSLKLSSVTAADTAVFYCARARSGITFGGVLVPGSFDIWGQGTMVTVSS

SEQ ID NO: 187 - DNA, 24 bp, synthetic construct
  misc_feature 1..24: note = Synthetic
  source 1..24: mol_type = other DNA
ggtggctccttcagcagtcacttc

SEQ ID NO: 188 - protein, 8 aa, synthetic construct
  REGION 1..8: note = Synthetic
GGSFSSHF

SEQ ID NO: 189 - DNA, 21 bp, synthetic construct
  misc_feature 1..21: note = Synthetic
  source 1..21: mol_type = other DNA
atctattacagtgggggcacc

SEQ ID NO: 190 - protein, 7 aa, synthetic construct
  REGION 1..7: note = Synthetic
IYYSGGT

SEQ ID NO: 191 - DNA, 60 bp, synthetic construct
  misc_feature 1..60: note = Synthetic
  source 1..60: mol_type = other DNA
gcgagagctagatcggggataacgtttgggggagttctcgtccctggttcttttgatatt

SEQ ID NO: 192 - protein, 20 aa, synthetic construct
  REGION 1..20: note = Synthetic
ARARSGITFGGVLVPGSFDI

SEQ ID NO: 193 - DNA, 378 bp, synthetic construct
  misc_feature 1..378: note = Synthetic
  source 1..378: mol_type = other DNA
caggtgcagctgcaggagtcgggcccaggactggtgaagccttcggagaccctgtccctcacctgcactgtctctggtggctccttcagtagtcacttctggagctggatccggcagcccccaggaaagggactggagtggattgggtatatctattacagtgggggcacccactacaacccctccctcgagagtcgagtcaccatatcagtagacacgtccaagaaccagttctccctgaaactgaactctgtgaccgctgcggacacggccgtttattactgtgcgagagctagatcggggattacttttgggggacttatcgtccctggttcttttgatatctggggccaagggacaatggtcaccgtctcttca

SEQ ID NO: 194 - protein, 126 aa, synthetic construct
  REGION 1..126: note = Synthetic
QVQLQESGPGLVKPSETLSLTCTVSGGSFSSHFWSWIRQPPGKGLEWIGYIYYSGGTHYNPSLESRVTISVDTSKNQFSLKLNSVTAADTAVYYCARARSGITFGGLIVPGSFDIWGQGTMVTVSS

SEQ ID NO: 195 - DNA, 24 bp, synthetic construct
  misc_feature 1..24: note = Synthetic
  source 1..24: mol_type = other DNA
ggtggctccttcagtagtcacttc

SEQ ID NO: 196 - protein, 8 aa, synthetic construct
  REGION 1..8: note = Synthetic
GGSFSSHF

SEQ ID NO: 197 - DNA, 21 bp, synthetic construct
  misc_feature 1..21: note = Synthetic
  source 1..21: mol_type = other DNA
atctattacagtgggggcacc

SEQ ID NO: 198 - protein, 7 aa, synthetic construct
  REGION 1..7: note = Synthetic
IYYSGGT

SEQ ID NO: 199 - DNA, 60 bp, synthetic construct; identical to SEQ ID NO: 143
  misc_feature 1..60: note = Synthetic
  source 1..60: mol_type = other DNA

SEQ ID NO: 200 - protein, 20 aa, synthetic construct
  REGION 1..20: note = Synthetic
ARARSGITFGGLIVPGSFDI

SEQ ID NO: 201 - DNA, 375 bp, synthetic construct
  misc_feature 1..375: note = Synthetic
  source 1..375: mol_type = other DNA
cgggtgcaactggtgcagtctgggtctgaggtgaagaagcctggggcctcagtgaaggtctcctgcagggcttctggatacatcttcaccagttatgatatcaattgggtgcgacaggccactggacaagggcttgagtggatgggatggatgaaccctaataatggtaacacagcctatacacagaagttccagggcagagtcaccatgaccaggaacacctccataagcacagcctacatggagctgagcagcctgagatctgaggacacggccgtgtattactgtgcgagaaagggattactatggttcgggaagttattagggtacggtatggacgtctggggccaagggaccacggtcaccgtctcctca

SEQ ID NO: 202 - protein, 125 aa, synthetic construct
  REGION 1..125: note = Synthetic
RVQLVQSGSEVKKPGASVKVSCRASGYIFTSYDINWVRQATGQGLEWMGWMNPNNGNTAYTQKFQGRVTMTRNTSISTAYMELSSLRSEDTAVYYCARKGLLWFGKLLGYGMDVWGQGTTVTVSS

SEQ ID NO: 203 - DNA, 24 bp, synthetic construct
  misc_feature 1..24: note = Synthetic
  source 1..24: mol_type = other DNA
ggatacatcttcaccagttatgat

SEQ ID NO: 204 - protein, 8 aa, synthetic construct
  REGION 1..8: note = Synthetic
GYIFTSYD

SEQ ID NO: 205 - DNA, 24 bp, synthetic construct
  misc_feature 1..24: note = Synthetic
  source 1..24: mol_type = other DNA
atgaaccctaataatggtaacaca

SEQ ID NO: 206 - protein, 8 aa, synthetic construct
  REGION 1..8: note = Synthetic
MNPNNGNT

SEQ ID NO: 207 - DNA, 54 bp, synthetic construct
  misc_feature 1..54: note = Synthetic
  source 1..54: mol_type = other DNA
gcgagaaagggattactatggttcgggaagttattagggtacggtatggacgtc

SEQ ID NO: 208 - protein, 18 aa, synthetic construct
  REGION 1..18: note = Synthetic
ARKGLLWFGKLLGYGMDV

SEQ ID NO: 209 - DNA, 324 bp, synthetic construct
  misc_feature 1..324: note = Synthetic
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccttggacgttcggccaagggaccaaggtggaaatcaaa

SEQ ID NO: 210 - protein, 108 aa, synthetic construct; identical to SEQ ID NO: 146
  REGION 1..108: note = Synthetic

SEQ ID NO: 211 - DNA, 21 bp, synthetic construct
  misc_feature 1..21: note = Synthetic
  source 1..21: mol_type = other DNA
cagagtgttagcagcagctac

SEQ ID NO: 212 - protein, 7 aa, synthetic construct
  REGION 1..7: note = Synthetic
QSVSSSY

SEQ ID NO: 213-214 - skipped (2 entries)

SEQ ID NO: 215 - DNA, 27 bp, synthetic construct
  misc_feature 1..27: note = Synthetic
  source 1..27: mol_type = other DNA
cagcagtatggtagctcaccttggacg

SEQ ID NO: 216 - protein, 9 aa, synthetic construct
  REGION 1..9: note = Synthetic
QQYGSSPWT

SEQ ID NO: 217 - protein, 120 aa, synthetic construct
  REGION 1..120: note = Synthetic
EVQVLESGGDLVQPGGSLRLSCAASGFTFSAYAMTWVRQAPGKGLEWVSAISGSGGSAYYADSVKGRFTISRDNSKNTVYLQMNSLRAEDTAVYYCAKDGAWKMSGLDVWGQGTTVIVSS

SEQ ID NO: 218 - protein, 8 aa, synthetic construct
  REGION 1..8: note = Synthetic
GFTFSAYA

SEQ ID NO: 219 - protein, 8 aa, synthetic construct
  REGION 1..8: note = Synthetic
ISGSGGSA

SEQ ID NO: 220 - protein, 13 aa, synthetic construct
  REGION 1..13: note = Synthetic
AKDGAWKMSGLDV

SEQ ID NO: 221 - protein, 107 aa, synthetic construct
  REGION 1..107: note = Synthetic
DIQMTQSPASLSASVGDRVTITCRASQDISDYLAWYQQKPGKIPRLLIYTTSTLQSGVPSRFRGSGSGTDFTLTISSLQPEDVATYYCQKYDSAPLTFGGGTKVEIK

SEQ ID NO: 222 - protein, 6 aa, synthetic construct
  REGION 1..6: note = Synthetic
QDISDY

SEQ ID NO: 223 - skipped

SEQ ID NO: 224 - protein, 9 aa, synthetic construct
  REGION 1..9: note = Synthetic
QKYDSAPLT

SEQ ID NO: 225 - protein, 109 aa, Homo sapiens
DFGLDCDEHSTESRCCRYPLTVDFEAFGWDWIIAPKRYKANYCSGECEFVFLQKYPHTHLVHQANPRGSAGPCCTPTKMSPINMLYFNGKEQIIYGKIPAMVVDRCGCS

SEQ ID NO: 226 - protein, 426 aa, Homo sapiens
MPLLWLRGFLLASCWIIVRSSPTPGSEGHSAAPDCPSCALAALPKDVPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 227 - protein, 340 aa, synthetic construct
  REGION 1..340: note = Synthetic
EAETRECIYYNANWELERTNQSGLERCEGEQDKRLHCYASWANSSGTIELVKKGCWLDDFNCYDRQECVATEENPQVYFCCCEGNFCNERFTHLPEAGGPEVTYEPPPTAPSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 228 - protein, 407 aa, Homo sapiens [sequence omitted]

(19) United States
(12) Patent Application Publication
(10) Pub. No.: US 2025/0277021 A1
(43) Pub. Date: Sep. 4, 2025

(54) Methods for Preventing and Treating Cardiac Dysfunction and COVID-19 with Activin A Antagonists

(71) Applicant: Regeneron Pharmaceuticals, Inc., Tarrytown, NY (US)

(72) Inventors: David Glass, Cortlandt Manor, NY (US); Scott Macdonnell, New Milford, CT (US); Jake Megna, Pine Bush, NY (US); Lori C. Morton, Chappaqua, NY (US)

(21) Appl. No.: 19/216,288

(22) Filed: May 22, 2025

Related U.S. Application Data
(63) Continuation of application No. 17/407,012, filed on Aug. 19, 2021, now abandoned.
(60) Provisional application No. 63/068,251, filed on Aug. 20, 2020.
(60) Provisional application No. 63/111,394, filed on Nov. 9, 2020.
(60) Provisional application No. 63/139,234, filed on Jan. 19, 2021.

(51) Int. Cl. C07K 16/22; A61K 39/00; A61P 9/00; A61P 31/14

(52) U.S. Cl. CPC C07K 16/22; A61P 9/00; A61P 31/14; A61K 2039/505; C07K 2317/21; C07K 2317/565; C07K 2317/76; C07K 2317/92

(57) ABSTRACT

The present invention provides methods for preventing and treating cardiac dysfunction, including cardiomyopathy and heart failure. The methods of the invention feature the administration of an antagonist of Activin A, e.g., a therapeutically effective amount of an antibody that binds to and reduces or neutralizes the activity of human Activin A. The methods of the invention are useful in preventing and treating cardiac disease from multiple causes, including viral disease, e.g., COVID-19.

### CROSS-REFERENCE TO RELATED APPLICATIONS

This application is a continuation of U.S. application Ser. No. 17/407,012, filed Aug. 19, 2021, which claims the benefit under 35 USC § 119 (e) of US Provisional Application Nos.: 63/068,251, filed Aug. 20, 2020; 63/111,394, filed Nov. 9, 2020; and 63/139,234, filed Jan. 19, 2021, each of which is incorporated herein by reference in its entirety for all purposes.

### REFERENCE TO A SEQUENCE LISTING

This application incorporates by reference a computer readable Sequence Listing in ST.26 XML format, titled 10771US02_Sequence, created on May 22, 2025 and containing 280,505 bytes.

### FIELD OF THE INVENTION

The present invention resides in the field of medicine, and relates to methods and pharmaceutical compositions for preventing and treating cardiac dysfunction with Activin A antagonists, including anti-Activin A antibodies and antigen-binding fragments thereof, or combinations of such antibodies or antigen-binding fragments and a myostatin inhibitor.

### BACKGROUND

Activins belong to the transforming growth factor-beta (TGF-β) superfamily and exert a broad range of biological effects on cell proliferation, differentiation, and apoptosis. Activins are homo- or heterodimers of InhibinβA, InhibinβB, InhibinβC and InhibinβE, different combinations of which create the various members of the activin protein group. For example, Activin A is a homodimer of InhibinβA and Activin B is a homodimer of InhibinβB, whereas Activin AB is a heterodimer of InhibinβA and InhibinβB and Activin AC is a heterodimer of InhibinβA and InhibinβC (Tsuchida, K. et al., Cell Commun Signal 7:15 (2009)).

Activin A binds to and activates receptor complexes on the surface of cells known as Activin Type II receptors (Type IIA and Type IIB, also known as ActRIIA and ActRIIB, respectively). The activation of these receptors leads to the phosphorylation of an Activin Type I receptor (e.g., Alk4 or 7), which in turn leads to the phosphorylation of SMAD 2 and 3 proteins, the formation of SMAD complexes (with SMAD4), and the translocation of the SMAD complex to the cell nucleus, where SMAD2 and SMAD3 function to regulate transcription of various genes (Sozzani, S. and Musso, T., Blood 117 (19): 5013-5015 (2011)).

Activin A or other ligands (including GDF8 (myostatin), Activin B, Activin AB, Inhibin A, Inhibin B, GDF3, GDF11, Nodal, BMP2, BMP4, BMP7, BMP9, and BMP10) that bind to and activate ActRIIB have been associated with a variety of conditions, including muscle wasting in aging and disease, and pulmonary and cardiac conditions. For example, overexpression of Activin A in mouse airways has been implicated in pulmonary pathology reminiscent of acute lung injury and acute respiratory distress syndome, which is attenuated via neutralization of Activin A with a fusion protein composed of the extracellular portion of the Activin type II receptor ActRIIB fused to the Fc portion of human IgG1 (Apostolou et al., Am J Respir Crit Care Med., 185 (4): 382-391). Similarly, Activin type II receptor (ActRII) ligands have been implicated in cardiac aging and heart failure. Inhibition of the ActRII pathway with an antibody (CDD866) that blocks ActRIIA and ActRIIB, or an ActRIIB-Fc fusion protein (RAP-031) that blocks pathway activation by binding circulating ActRII ligands, reduced cardiac ActRII signaling while restoring or preserving cardiac function (Roh et al., Sci. Transl. Med, 11, eaau8680, 2019).

Agents that bind to multiple ActRII ligands or generally inhibit ActRII signaling are known to cause various adverse effects when administered to human patients. The distinct roles of the many ActRII ligands have yet to be completely elucidated, and a need exists for specific inhibitors of ActRII ligands that can provide clinical benefits.

### BRIEF SUMMARY OF THE INVENTION

In one aspect, the present invention provides a method of preventing or treating cardiac dysfunction or heart failure in a subject in need thereof, the method comprising administering an Activin A specific antagonist to the subject.

In some embodiments, the Activin A specific antagonist is an anti-Activin A antibody or antigen-binding fragment thereof. In some cases, the antibody or antigen-binding fragment thereof specifically binds Activin A with a binding dissociation equilibrium constant (K_{D}) of less than about 5 pM as measured in a surface plasmon resonance assay at 25° C. In some cases, the antibody or antigen-binding fragment thereof specifically binds Activin A with a K_{D} of less than about 4 pM as measured in a surface plasmon resonance assay at 25° C. In some cases, the antibody or antigen-binding fragment thereof specifically binds Activin A with a binding association equilibrium constant (Kₐ) of less than about 500 nM.

In some embodiments, the antibody or antigen-binding fragment thereof blocks binding of at least one Activin A receptor to Activin A. In some embodiments, the antibody or antigen-binding fragment thereof blocks activation of at least one Activin A receptor by Activin A. In some cases, the antibody or antigen-binding fragment thereof does not significantly block binding of Activin A to an Activin Type II receptor. In some cases, the antibody or antigen-binding fragment thereof blocks Activin A binding to an Activin A receptor with an IC₅₀ value of less than about 80 pM as measured in an in vivo receptor/ligand binding bioassay at 25° C. In some cases, the antibody or antigen-binding fragment thereof blocks Activin A binding to an Activin A receptor with an IC₅₀ value of less than about 60 pM as measured in an in vivo receptor/ligand binding bioassay at 25° C.

In some embodiments, the antibody or antigen-binding fragment thereof inhibits binding of Activin A to an Activin A receptor selected from the group consisting of Activin Type IIA receptor (ActRIIA), Activin Type IIB receptor (ActRIIB), and Activin Type I receptor. In some embodiments, the antibody or antigen-binding fragment thereof inhibits Activin A-mediated activation of SMAD complex signaling.

In any of the various embodiments, the antibody or antigen-binding fragment comprises: (a) the complementarity determining regions (CDRs) of a heavy chain variable region (HCVR) comprising an amino acid sequence selected from the group consisting of SEQ ID NOs: 2, 18, 34, 50, 66, 82, 98, 106, 114, 122, 130, 138, 154, 162, 170, 178, 186, 194, and 202; and (b) the CDRs of a light chain variable region (LCVR) comprising an amino acid sequence selected from the group consisting of SEQ ID NOs: 10, 26, 42, 58, 74, 90, 146, and 210. In some embodiments, the antibody or antigen-binding fragment comprises the heavy and light chain CDRs of a HCVR/LCVR amino acid sequence pair selected from the group consisting of: SEQ ID NOs: 2/10, 18/26, 34/42, 50/58, 66/74, 82/90, 98/90, 106/90, 114/90, 122/90, 130/90, 138/146, 154/146, 162/146, 170/146, 178/146, 186/146, 194/146, and 202/210. In some embodiments, the antibody or antigen-binding fragment thereof comprises HCDR1-HCDR2-HCDR3-LCDR1-LCDR2-LCDR3 domains comprising the amino acid sequences, respectively, selected from the group consisting of: SEQ ID NOs: 4-6-8-12-14-16; 20-22-24-28-30-32; 36-38-40-44-46-48; 52-54-56-60-62-64; 68-70-72-76-78-80; 84-86-88-92-94-96; 100-102-104-92-94-96; 108-110-112-92-94-96; 116-118-120-92-94-96; 124-126-128-92-94-96; 132-134-136-92-94-96; 140-142-144-148-150-152; 156-158-160-148-150-152; 164-166-168-148-150-152; 172-174-176-148-150-152; 180-182-184-148-150-152; 188-190-192-148-150-152; 196-198-200-148-150-152; and 204-206-208-212-214-216.

In any of the various embodiments, the antibody or antigen-binding fragment comprises: (a) a HCVR comprising an amino acid sequence selected from the group consisting of SEQ ID NOs: 2, 18, 34, 50, 66, 82, 98, 106, 114, 122, 130, 138, 154, 162, 170, 178, 186, 194, and 202; and (b) a LCVR comprising an amino acid sequence selected from the group consisting of SEQ ID NOs: 10, 26, 42, 58, 74, 90, 146, and 210. In some embodiments, the antibody or antigen-binding fragment comprises a HCVR/LCVR amino acid sequence pair selected from the group consisting of: SEQ ID NOs: 2/10, 18/26, 34/42, 50/58, 66/74, 82/90, 98/90, 106/90, 114/90, 122/90, 130/90, 138/146, 154/146, 162/146, 170/146, 178/146, 186/146, 194/146, and 202/210.

In one aspect, the present invention provides a method of preventing or treating cardiac dysfunction or heart failure in a subject in need thereof, the method comprising administering an antibody that specifically binds Activin A or an antigen-binding fragment thereof, wherein the antibody or antigen-binding fragment thereof comprises HCDR1-HCDR2-HCDR3-LCDR1-LCDR2-LCDR3 domains comprising the amino acid sequences, respectively, of SEQ ID NOs: 68-70-72-76-78-80. In some embodiments, the antibody or antigen-binding fragment comprises a HCVR comprising the amino acid sequence of SEQ ID NO: 66, and a LCVR comprising the amino acid sequence of SEQ ID NO: 74.

In one aspect, the present invention provides a method of preventing or treating cardiac dysfunction or heart failure in a subject in need thereof, the method comprising administering an antibody that specifically binds Activin A or an antigen-binding fragment thereof, wherein the antibody or antigen-binding fragment thereof comprises HCDR1-HCDR2-HCDR3-LCDR1-LCDR2-LCDR3 domains comprising the amino acid sequences, respectively, of SEQ ID NOs: 164-166-168-148-150-152. In some embodiments, the antibody or antigen-binding fragment comprises a HCVR comprising the amino acid sequence of SEQ ID NO: 162, and a LCVR comprising the amino acid sequence of SEQ ID NO: 146.

In any of the various embodiments, the antibody or antigen-binding fragment may be a human antibody comprising an IgG heavy chain constant region. In some embodiments, the IgG heavy chain constant region is of IgG1 isotype. In some embodiments, the IgG heavy chain constant region is of IgG4 isotype.

In any of the various embodiments, the method further comprises administration of the antibody or antigen-binding fragment in combination with a GDF8 antagonist. In some embodiments, the GDF8 antagonist is selected from the group consisting of a GDF8-inhibiting fusion protein, an anti-GDF8 antibody, and an antigen-binding fragment of an anti-GDF8 antibody. In some cases, the GDF8 antagonist is an anti-GDF8 antibody or antigen-binding fragment thereof. In some embodiments, the anti-GDF8 antibody or antigen-binding fragment thereof comprises the CDRs of a HCVR comprising the amino acid sequence of SEQ ID NO:217, and the CDRs of a LCVR comprising the amino acid sequences of SEQ ID NO:221. In some embodiments, the anti-GDF8 antibody or antigen-binding fragment thereof comprises HCDR1-HCDR2-HCDR3-LCDR1-LCDR2-LCDR3 domains comprising the amino acid sequences, respectively, of SEQ ID NOs: 218-219-220-222-223-224. In some embodiments, the anti-GDF8 antibody or antigen-binding fragment thereof comprises a HCVR comprising the amino acid sequence of SEQ ID NO: 217, and a LCVR comprising the amino acid sequence of SEQ ID NO: 221.

In any of the various embodiments discussed above or herein, the subject has been diagnosed with a viral infection. In some embodiments, the viral infection is a coronavirus infection. In some cases, the coronavirus is severe acute respiratory syndrome coronavirus 2 (SARS-COV-2). In some cases, the subject has severe COVID-19 symptoms. In some cases, the subject has critical COVID-19 symptoms.

In another aspect, the present invention provides a pharmaceutical composition comprising an Activin A specific antagonist (e.g., a recombinant human anti-Activin A antibody or antigen-binding fragment fragment thereof, as discussed above or herein), and a pharmaceutically acceptable carrier, for preventing or treating cardiac dysfunction or heart failure in a subject in need thereof.

In another aspect, the present invention provides an Activin A specific antagonist (e.g., an anti-Activin A antibody or antigen-binding fragment thereof, as discussed above or herein), for use in a method of preventing or treating cardiac dysfunction or heart failure in a subject in need thereof.

In another aspect, the present invention provides a method of treating COVID-19 in a subject that has tested positive for a severe acute respiratory syndrome coronavirus 2 (SARS-CoV-2) infection, the method comprising administering an Activin A specific antagonist to the subject. In some embodiments, the Activin A specific antagonist is an anti-Activin A antibody or antigen-binding fragment thereof.

In some cases, the antibody or antigen-binding fragment comprises the heavy and light chain CDRs of a HCVR/LCVR amino acid sequence pair selected from the group consisting of: SEQ ID NOs: 2/10, 18/26, 34/42, 50/58, 66/74, 82/90, 98/90, 106/90, 114/90, 122/90, 130/90, 138/146, 154/146, 162/146, 170/146, 178/146, 186/146, 194/146, and 202/210. In some cases, the antibody or antigen-binding fragment thereof comprises HCDR1-HCDR2-HCDR3-LCDR1-LCDR2-LCDR3 domains comprising the amino acid sequences, respectively, selected from the group consisting of: SEQ ID NOs: 4-6-8-12-14-16; 20-22-24-28-30-32; 36-38-40-44-46-48; 52-54-56-60-62-64; 68-70-72-76-78-80; 84-86-88-92-94-96; 100-102-104-92-94-96; 108-110-112-92-94-96; 116-118-120-92-94-96; 124-126-128-92-94-96; 132-134-136-92-94-96; 140-142-144-148-150-152; 156-158-160-148-150-152; 164-166-168-148-150-152; 172-174-176-148-150-152; 180-182-184-148-150-152; 188-190-192-148-150-152; 196-198-200-148-150-152; and 204-206-208-212-214-216. In some cases, the antibody or antigen-binding fragment comprises: (a) a HCVR comprising an amino acid sequence selected from the group consisting of SEQ ID NOs: 2, 18, 34, 50, 66, 82, 98, 106, 114, 122, 130, 138, 154, 162, 170, 178, 186, 194, and 202; and (b) a LCVR comprising an amino acid sequence selected from the group consisting of SEQ ID NOs: 10, 26, 42, 58, 74, 90, 146, and 210. In some cases, the antibody or antigen-binding fragment comprises a HCVR/LCVR amino acid sequence pair selected from the group consisting of: SEQ ID NOs: 2/10, 18/26, 34/42, 50/58, 66/74, 82/90, 98/90, 106/90, 114/90, 122/90, 130/90, 138/146, 154/146, 162/146, 170/146, 178/146, 186/146, 194/146, and 202/210. In some cases, the antibody or antigen-binding fragment thereof comprises HCDR1-HCDR2-HCDR3-LCDR1-LCDR2-LCDR3domains comprising the amino acid sequences, respectively, of SEQ ID NOs: 68-70-72-76-78-80. In some cases, the antibody or antigen-binding fragment comprises a HCVR comprising the amino acid sequence of SEQ ID NO: 66, and a LCVR comprising the amino acid sequence of SEQ ID NO: 74. In some cases, the antibody or antigen-binding fragment thereof comprises HCDR1-HCDR2-HCDR3-LCDR1-LCDR2-LCDR3 domains comprising the amino acid sequences, respectively, of SEQ ID NOs: 164-166-168-148-150-152. In some cases, the antibody or antigen-binding fragment comprises a HCVR comprising the amino acid sequence of SEQ ID NO: 162, and a LCVR comprising the amino acid sequence of SEQ ID NO: 146.

In some embodiments, the antibody or antigen-binding fragment is a human antibody comprising an IgG heavy chain constant region. In some cases, the IgG heavy chain constant region is of IgG1 isotype. In some cases, the IgG heavy chain constant region is of IgG4 isotype.

In some embodiments, the subject has severe COVID-19 symptoms requiring supplemental oxygen administration. In some embodiments, the subject has critical COVID-19 symptoms requiring mechanical ventilation or treatment in an intensive care unit.

In another aspect, the present invention provides for use of an Activin A specific antagonist (e.g., an anti-Activin A antibody or antigen-binding fragment thereof, as discussed above or herein), in the manufacture of a medicament for preventing or treating cardiac dysfunction or heart failure in a subject in need thereof, or for treating a COVID-19 patient.

In various embodiments, any of the features or components of embodiments discussed above or herein may be combined, and such combinations are encompassed within the scope of the present disclosure. Any specific value discussed above or herein may be combined with another related value discussed above or herein to recite a range with the values representing the upper and lower ends of the range, and such ranges are encompassed within the scope of the present disclosure.

Other embodiments will become apparent from a review of the ensuing detailed description.

### BRIEF DESCRIPTION OF THE FIGURES

FIGS. 1A and 1B are graphs showing the negative effects of Activin A on impedence amplitude of human induced pluripotent stem cells in culture following a single treatment with Activin A (FIG. 1A) or multiple treatments of Activin A (FIG. 1B).

FIG. 2 is a graph showing the positive effects of an anti-Activin A antibody (mAb1) on preventing Activin A-mediated cardiac dysfunction in human induced pluripotent stem cells.

FIG. 3 is a series of graphs showing the relative increases of Activin A, follistatin-related gene (FLRG) and plasminogen activator inhibitor-1 (PAI-1) in serum samples from COVID-19 patients compared to controls.

FIG. 4 is a set of graphs showing the correlation between disease severity and serum levels of Activin A and FLRG in COVID-19 patients.

FIG. 5 is a set of graphs showing the relative levels of Activin A and FLRG in serum samples of various age groups of COVID-19 patients compared to healthy aged-matched controls.

FIG. 6 is a graph showing the correlation between disease severity and serum levels of PAI-1 in COVID-19 patients.

FIG. 7 is a set of graphs showing the correlation between disease severity and serum levels of PAI-1 in male (left panel) and female (right panel) COVID-19 patients.

FIG. 8 is a graph showing the relative level of PAI-1 in serum samples of various age groups of COVID-19 patients compared to healthy aged-matched controls.

FIG. 9 is a set of graphs showing that treatment with corticosteroids did not significantly affect serum levels of Activin A and FLRG in patients with severe or critical COVID-19 symptoms.

FIG. 10 is a set of graphs showing activation of gene markers of cardiac stress (NPPA—atrial natriuretic peptide, and NPPB—B-type natriuretic peptide) and Activin A signaling genes (FSTL3—follistatin like 3 protein, also known as FLRG, and Serpine1, also known as PAI-1) in IPSC-cardiomyocytes treated with Activin A.

FIG. 11 is a graph showing that the IKK/NFκB pathway is mainly responsible for Activin A induction by IL1β and TNFα.

FIG. 12 is a Western blot and graph showing an increase in SMAD2/3 phosphorylation in human inducible pluripotent stem cell-derived (IPSC) cardiomyocytes exposed to Activin A, and the blockade of this increase in SMAD2/3 phosphorylation with an inhibitory anti-Activin A antibody (mAb2). The Control mAb is an antibody that binds an irrelevant, non-human antigen.

FIGS. 13A and 13B are graphs showing an elongated action potential, a reduction in field potential amplitude, and a reduced field potential downstroke velocity in cardiomyocytes chronically exposed to Activin A, and prevention of these effects in the presence of an inhibitory anti-Activin A antibody (mAb1).

FIGS. 14A and 14B are graphs showing reduced peak calcium flux amplitude, increased calcium flux falling time, and increased calcium flux rising time in cardiomyocytes chronically exposed to Activin A, and prevention of these effects in the presence of an inhibitory anti-Activin A antibody (mAb1).

### DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present invention, the preferred methods and materials are now described. All patents, applications and non-patent publications mentioned in this specification are incorporated herein by reference in their entireties.

### Methods of Preventing and Treating Cardiac Dysfunction and Heart Failure

The present invention provides methods for preventing and treating cardiac dysfunction and heart failure. In some embodiments, the present invention provides methods for treating, preventing and reducing the severity or progression of heart failure or one or more complications of heart failure. In some embodiments, the present invention provides methods for improving human cardiomyocyte function, including contractility and electrical properties.

As discussed herein, it has been discovered that an Activin A specific antagonist (e.g., an anti-Activin A antibody or antigen-binding fragment thereof) provides a surprising effect on treating and preventing various compilations of cardiac dysfunction and heart failure. For example, anti-Activin A antibodies can be used to prevent or reduce the severity of cardiac hypertrophy, cardiac remodeling, and cardiac fibrosis as well as improve cardiac function in a transverse aortic constriction (TAC) heart failure model. In addition, Activin A specific antagonist treatment can increase survival time of heart failure patients. Accordingly, the disclosure provides, in part, methods of using Activin A specific antagonists (e.g., an anti-Activin A antibodies or antigen-binding fragment thereof), alone or in combination with one or more additional supportive therapies and/or additional active agents, to treat, prevent, or reduce the severity of heart failure, particularly treating, preventing, or reducing the severity of one or more complications of a heart failure (e.g., cardiac hypertrophy, cardiac remodeling, and cardiac fibrosis) as well as improving cardiac function and increasing survival time of heart failure patients.

As used herein, a therapeutic agent that “prevents” a disorder or condition refers to a compound that, in a statistical sample, reduces the occurrence of the disorder or condition in the treated sample relative to an untreated control sample or delays the onset of the disorder or condition relative to the untreated control sample. The term “treating” as used herein includes amelioration or elimination of the condition once it has been established. In either case, prevention or treatment may be discerned in the diagnosis provided by a physician or other health care provider and the intended result of administration of the therapeutic agent.

In general, treatment or prevention of a disease or condition as described in the present disclosure is achieved by administering one or more Activin A specific antagonists (e.g., an anti-Activin A antibody or antigen-binding fragment thereof) in an effective amount. An effective amount of an agent refers to an amount effective, at dosages and for periods of time necessary, to achieve the desired therapeutic or prophylactic result. A therapeutically effective amount of an agent of the present disclosure may vary according to factors such as the disease state, age, sex, and weight of the individual, and the ability of the agent to elicit a desired response in the individual. A prophylactically effective amount refers to an amount effective, at dosages and for periods of time necessary, to achieve the desired prophylactic result.

Heart failure is a clinical syndrome defined by typical symptoms and signs resulting from certain structural or functional abnormality of the heart (ESC Guidelines for the diagnosis and treatment of acute and chronic heart failure. McMurray J J et al. European Heart Journal 2012, 14 (8): 803-69; 2013 ACCF/AHA Guideline for the Management of Heart Failure, Yanzy C W et al. Circulation 2013, 128, e240-e327). For example, cardiac abnormalities may impair the ability to fill or eject blood, and/or lead to failure to deliver sufficient oxygen to meet the requirements of the metabolizing tissues, despite normal filling pressures, or only at the expense of increased filling pressures. As used herein, the term heart failure encompasses a variety cardiovascular conditions which include, but are not limited to, heart failure due to left ventricular dysfunction, heart failure with normal ejection fraction, heart failure due to aortic stenosis, acute heart failure, chronic heart failure, congestive heart failure, congenital heart failure, compensated heart failure, decompensated heart failure, diastolic heart failure, systolic heart failure, right-side heart (ventricle) failure, left-side heart (ventricle) failure, biventricular heart failure, forward heart failure, backward heart failure, high output heart failure, low output heart failure. Also heart failure includes heart conditions relating to fluid build-up in the heart, such as myocardial edema.

In general, clinical manifestations of heart failure include, for example, dyspnea (shortness of breath), orthopnea, paroxysmal nocturnal dyspnea, and fatigue (which may limit exercise tolerance), fluid retention (which may lead to, for example, pulmonary congestion and peripheral edema), angina, hypertension, arrhythmia, ventricular arrhythmias, cardiomyopathy, cardiac hypertrophy, cardiac asthma, nocturia, ascities, congestive hepatopathy, coagulopathy, reduced renal blood flow, renal insufficiency, myocardial infarction, and stroke.

Although the phrase “congestive heart failure” is often used to describe all types of heart failure, including the above listed types, congestive heart failure is more accurately descriptive of a symptom of heart failure relating to pulmonary congestion or fluid buildup in the lungs. This congestion is more commonly symptom of systolic and left-sided heart failure. As the efficiency of the pulmonary system declines, increased blood volume near the input side of the heart changes the pressure at the alveolar arterial interface, an interface between the lung capillaries and the alveolar space of the lungs. The change in pressure at the interface causes blood plasma to push out into the alveolar space in the lungs. Dyspnea and general fatigue are typical perceived manifestations of congestive heart failure.

There are many different ways to categorize heart failure. For example, heart failure may be characterized based on the side of the heart involved (left heart failure versus right heart failure). Right heart failure compromises pulmonary flow to the lungs. Left heart failure compromises aortic flow to the body and brain. Mixed presentations are common; left heart failure often leads to right heart failure in the longer term. Heart failure also may be classified on whether the abnormality is due to insufficient contraction (systolic dysfunction; systolic heart failure), or due to insufficient relaxation of the heart (diastolic dysfunction; diastolic heart failure), or to both. In addition, heart failure may be classified on whether the problem is primarily increased venous back pressure (preload), or failure to supply adequate arterial perfusion (afterload). Heart failure may be classified on whether the abnormality is due to low cardiac output with high systemic vascular resistance or high cardiac output with low vascular resistance (low-output heart failure vs. high-output heart failure). Also, heart failure may be classified based on the degree of coexisting illness, for example, heart failure/systemic hypertension, heart failure/pulmonary hypertension, heart failure/diabetes, and heart failure/kidney failure.

Furthermore, heart failure may be classified based on the degree of functional impairment conferred by the cardiac abnormality. Functional classification generally relies on the New York Heart Association (NYHA) functional classification. The classes (I-IV) are: class I: no limitation is experienced in any activities; there are no symptoms from ordinary activities; class II: slight, mild limitation of activity; the patient is comfortable at rest or with mild exertion; class III: marked limitation of any activity; the patient is comfortable only at rest; and class IV: any physical activity brings on discomfort and symptoms occur at rest. This score documents the severity of symptoms and can be used to assess response to treatment.

In its 2001 guidelines the American College of Cardiology/American Heart Association (ACC) working group introduced four stages of heart failure [see, e.g., Hunt, S., “ACC/AHA 2005 Guideline Update for the Diagnosis and Management of Chronic Heart Failure in the Adult: A Report of the American College of Cardiology/American Heart Association Task Force on Practice Guidelines (Writing Committee to Update the 2001 Guidelines for the Evaluation and Management of Heart Failure),” J. Am, Coll. Cardiol, 46: e1-e82 (2005]. The first stage, Stage A, is a subject at high risk for heart failure but without structural heart disease or symptoms of heart failure (for example, these are patients with hypertension, atherosclerotic disease, diabetes, obesity, metabolic syndrome or patients using cardiotoxins). The second stage, Stage B, is a subject having structural heart disease but without signs or symptoms of heart failure (for example, these are patients who have previously had a myocardial infarction, exhibit cardiac remodeling including hypertrophy and low ejection fraction, and patients with asymptomatic valvular disease). The third stage, Stage C, is a subject having structural heart disease with prior or current symptoms of heart failure (for example, these are patients who have known structural heart disease and exhibit shortness of breath and fatigue and have reduced exercise tolerance). The fourth and final stage, Stage D, is refractory heart failure requiring specialized interventions (for example, patients who have marked symptoms at rest despite maximal medical therapy (namely, those who are recurrently hospitalized or cannot be safely discharged from the hospital without specialized interventions). The ACC staging system is useful in that Stage A encompasses “pre-heart failure”—a stage where intervention with treatment can presumably prevent progression to overt symptoms. ACC Stage A does not have a corresponding NYHA class. ACC Stage B would correspond to NYHA Class I. ACC Stage C corresponds to NYHA Class Il and III, while ACC Stage D overlaps with NYHA Class IV.

Cardiac remodeling, which usually precedes clinical signs of heart failure, refers to the molecular, cellular and/or interstitial changes manifested clinically as changes in size, shape and function of the heart generally resulting from cardiac load or injury (Cohn J N et al. JACC 2000. 35 (3): 569-82). Triggers for cardiac remodeling include, for example, myocardial infarction, hypertension, wall stress, inflammation, pressure overload, and volume overload. Alterations in myocardial structure can occur as quickly as within a few hours of injury and may progress over months and years. While initially beneficial, these changes can impair myocardial function to the point of chronic intractable heart failure over time (months to years). Hallmarks of cardiac remodeling include, for example, chamber dilation, increase in ventricular sphericity, and development of interstitial and perivascular fibrosis. Increased sphericity is positively associated with mitral regurgitation. Ventricular dilation mainly results from cardiomyocyte hypertrophy and lengthening and to a lesser extent from increases in the ventricular mass.

In some embodiments, Activin A specific antagonists of the disclosure (e.g., an anti-Activin A antibody or antigen-binding fragment thereof) may be used to treat, prevent, or reduce the progression of cardiac remodeling. For example, Activin A specific antagonists may be used to maintain myocardial structure or decrease alterations in myocardial structure of the heart in a subject. Progression of cardiac remodeling can be assessed by comparing the alterations in myocardial structure of the heart over a period of time between two groups of subjects, in which a first group (the treatment group) is treated by the methods of the present invention, and a second group (the placebo group) is treated by using a placebo in replacement or in lieu of the treatment by the methods of the present invention. If the alterations in myocardial structure of the heart in the subjects of the treatment group are less than the alterations in myocardial structure of the heart in the subjects of the placebo group, then a determination is made that there has been a reduction in the progression of cardiac remodeling. Methods for determining disease progression or development, such as cardiac remodeling, can be assessed using well known methods including, for example, physical examination, 2-dimensional echocardiogram coupled with Doppler flow studies, ultrasound, MRI, computerized tomography, cardiac catheterization, radionuclide imaging (such as radionuclide ventriculography) as well as any combinations thereof.

In general, cardiac remodeling and heart failure result from disorders and conditions that cause persistent increase in cardiac workload or injury. Disorders and conditions leading to heart failure include, for example, loss of viable myocardium after myocardial infarction, coronary artery disease, hypertension, cardiomyopathies (e.g., dilated cardiomyopathy, cardiomyopathy from infections or alcohol/drug abuse, etc.), heart valve disease and dysfunction including, for example, aortic valve diseases (e.g., aortic valve insufficiency, aortic valve regurgitation, and aortic stenosis (aortic valve stenosis)), pulmonary disorders (e.g., pulmonary hypertension), congenital heart defects, acute ischemic injury, reperfusion injury, pericardium disorders and abnormalities, myocardium disorders, great vessels disorders, endocardium disorders, atrial fibrillation, impairment of left ventricular myocardial function, impairment of right ventricular myocardial function, cardiac arrhythmias, thyroid disease, kidney disease, diabetes, weakening of the heart muscle which leave it unable to pump enough blood, thyroid disease, neurohormonal imbalances, viral infections, and anemia. As such disorders and conditions may lead to cardiac remodeling and/or heart failure, subjects having, or suspected of having, one or more of these conditions are preferred subjects for treatment with one or more Activin A specfiic antagonists (e.g., an anti-Activin A antibody or antigen-binding fragment thereof), optionally in combination with one or more additional active agents or supportive therapies for treating cardiac remodeling and/or heart failure, in accordance with the present invention. In some embodiments, subjects with signs of cardiac remodeling (e.g., myocardial hypertrophy and ventricular dilation) or with overt heart failure, even when the underlying etiology cannot be detected, are also suitable for treatment in accordance with the present disclosure as preventing further cardiac remodeling or treating existing cardiac remodeling or reducing cardiac remodeling would be beneficial in these subjects. In some embodiments, subjects with risk factors for cardiac remodeling and/or heart failure development (e.g., subjects with those conditions that may lead to cardiac remodeling and/or chronic heart failure described herein) are also suitable for treatment in accordance with the present disclosure.

In general, hypertension or high blood pressure refers to a resting blood pressure, as measured with, for example, a sphygmomanometer, of greater than 120 mmHg (systolic)/80 mmHg (diastolic). Blood pressure between 121-139/81-89 is considered prehypertension and above this level (140/90 mm Hg or higher) is considered high (hypertension). Unless otherwise indicated, both prehypertension and hypertension blood pressure are included in the meaning of “hypertension” as used herein. For example, resting blood pressures of 135 mmHg/87 or of 140 mmHg/90 mmHg are intended to be within the scope of the term “hypertension” even though the 135/87 is generally considered within a prehypertensive category. Blood pressures of 145 mm Hg/90 mmHg, 140 mmHg/95 mmHg, and 142 mmHg/93 mmHg are further examples of high blood pressures. It will be appreciated that blood pressure normally varies throughout the day. It can even vary slightly with each heartbeat. Normally, it increases during activity and decreases at rest. It's often higher in cold weather and can rise when under stress. More accurate blood pressure readings can be obtained by daily monitoring blood pressure, where the blood pressure reading is taken at the same time each day to minimize the effect that external factors. Several readings over time may be needed to determine whether blood pressure is high. In general chronic hypertension refers to a subject which exhibits hypertension either continuously or intermittently for an extended period of time, such as, but not limited to at least one week, at least two weeks, at least three weeks, at least four weeks, at least two months, at least six months, at least one year, at least two years, at least three years, at least four years, at least five years, at least 10 years, etc.

In general, cardiac arrhythmia refers to a condition where the muscle contraction of the heart becomes irregular. An unusually fast rhythm (e.g., more than 100 beats per minute) is called tachycardia. An unusually slow rhythm (e.g., fewer than 60 beats per minute) is called bradycardia.

In general, cardiac hypertrophy refers to cardiac enlargement, a condition characterized by an increase in the size of heart and the individual cardiac muscle cells, particularly ventricular muscle cells, and an increase in the size of the inside cavity of a chamber of the heart.

Ejection fraction is the percentage of blood pumped out of the left ventricle with each heartbeat. Ejection fraction may be measured, for example, during an echocardiogram. Ejection fraction is an important measurement of how well a heart is pumping and can be used to classify heart failure and to guide treatment. Heart failure can be classified as heart failure with preserved ejection fraction (also referred to as diastolic heart failure) or as heart failure with reduced ejection fraction (also referred to as systolic heart failure). A recent study demonstrated that the prevalence of heart failure with preserved ejection fraction increased over a 15-year period, with no marked improvement in the mortality rates. If these trends continue, heart failure with preserved ejection fraction may become the most common form of heart failure, demonstrating a growing public health problem (Owan et al., 2006, N Engl J Med; 355 (3): 251-9).

In some embodiments, Activin A specific antagonists of the disclosure may be used to reduce the incidences of non-fatal or fatal cardiovascular events (e.g., myocardial infarction, stroke, angina, arrhythmias, fluid retention, and progression of heart failure). As used herein, reducing the incidences of cardiovascular events refers to maintaining or reducing the number of cardiovascular events experienced by a subject during or over the course of a period of time. A reduction in the incidence of cardiovascular events can be assessed or determined by comparing the incidences of cardiovascular events over or during the course of a period of time between two groups of subjects, in which a first group (the treatment group) is treated by the methods of the present invention, and a second group (the placebo group) is treated by using a placebo (namely, dummy pills) in replacement or in lieu of the treatment by the methods of the present invention. If the number of cardiovascular events for the treatment group is less than the number of the cardiovascular events for the placebo group, then a determination is made that there was or has been a reduction in the incidences of cardiovascular events. Alternatively, a reduction in the incidence of cardiovascular events can be assessed or determined by determining a baseline number of cardiovascular events for a subject population at a first period in time and then measuring the number of cardiovascular events for a subject population at a second, later period in time. If the number of cardiovascular events for the subject population at the second, later period in time is the same as or less then the number of cardiovascular events for the subject population at the first period in time, then a determination is made that there has been a reduction in the incidences of cardiovascular events for said subject population.

In some embodiments, Activin A specific antagonists of the disclosure (e.g., an anti-Activin A antibody or antigen-binding fragment thereof) may be used to reduce incidence of hospitalizations for heart failure. As used herein, reducing the incidences of hospitalizations for heart failure refers to maintaining or reducing the number of hospitalizations for heart failure experienced by a subject during or over the course of a period of time. A reduction in the incidence of hospitalizations for heart failure can be assessed or determined by comparing the incidences of hospitalizations for heart failure over or during the course of a period of time between two groups of subjects, in which a first group (the treatment group) is treated by the methods of the present invention, and a second group (the placebo group) is treated by using a placebo (namely, dummy pills) in replacement or in lieu of the treatment by the methods of the present invention. If the number of hospitalizations for heart failure for the treatment group is less than the number of the hospitalizations for heart failure for the placebo group, then a determination is made that there was or has been a reduction in the incidences of hospitalizations for heart failure. Alternatively, a reduction in the incidence of hospitalizations for heart failure can be assessed or determined by determining a baseline number of hospitalizations for heart failure for a subject population at a first period in time and then measuring the number of hospitalizations for heart failure for a subject population at a second, later period in time. If the number of hospitalizations for heart failure for the subject population at the second, later period in time is the same as or less then the number of hospitalizations for heart failure for the subject population at the first period in time, then a determination is made that there has been a reduction in the incidences of hospitalizations for heart failure for said subject population.

In some embodiments, Activin A specific antagonists of the disclosure (e.g., an anti-Activin A antibody or antigen-binding fragment thereof) may be used to improve survival of heart failure patients. As used herein, improving survival of heart failure patients refers to maintaining or reducing the number of fatal cardiovascular events experienced by a subject population during or over the course of a period of time. An improvement in survival of heart failure patients can be assessed or determined by comparing the incidences of fatal cardiovascular events over or during the course of a period of time between two groups of subjects, in which a first group (the treatment group) is treated by the methods of the present invention, and a second group (the placebo group) is treated by using a placebo (namely, dummy pills) in replacement or in lieu of the treatment by the methods of the present invention. If the number of fatal cardiovascular events for the treatment group is less than the number of the fatal cardiovascular events for the placebo group, then a determination is made that there was or has been an improvement in survival of heart failure patients. Alternatively, a reduction in the incidence of fatal cardiovascular events can be assessed or determined by determining a baseline number of fatal cardiovascular events for a subject population at a first period in time and then measuring the number of fatal cardiovascular events for a subject population at a second, later period in time. If the number of fatal cardiovascular events for the subject population at the second, later period in time is the same as or less then the number of fatal cardiovascular events for the subject population at the first period in time, then a determination is made that there has been an improvement in survival of heart failure patients for said subject population.

In some embodiments, Activin A specific antagonists of the disclosure (e.g., an anti-Activin A antibody or antigen-binding fragment thereof) may be used to reduce risk of cardiovascular death in heart failure patients. As used herein, reducing risk of cardiovascular death of heart failure patients refers to maintaining or reducing the number of fatal cardiovascular events experienced by a subject population during or over the course of a period of time. A reduction in cardiovascular deaths in heart failure patients can be assessed or determined by comparing the incidences of fatal cardiovascular events over or during the course of a period of time between two groups of subjects, in which a first group (the treatment group) is treated by the methods of the present invention, and a second group (the placebo group) is treated by using a placebo (namely, dummy pills) in replacement or in lieu of the treatment by the methods of the present invention. If the number of fatal cardiovascular events for the treatment group is less than the number of the fatal cardiovascular events for the placebo group, then a determination is made that there was or has been a reduction in cardiovascular deaths in heart failure patients. Alternatively, a reduction in cardiovascular deaths in heart failure patients can be assessed or determined by determining a baseline number of fatal cardiovascular events for a subject population at a first period in time and then measuring the number of fatal cardiovascular events for a subject population at a second, later period in time. If the number of fatal cardiovascular events for the subject population at the second, later period in time is the same as or less then the number of fatal cardiovascular events for the subject population at the first period in time, then a determination is made that there has been a reduction in cardiovascular deaths in heart failure patients for said subject population.

In some embodiments, Activin A specific antagonists of the disclosure (e.g., an anti-Activin A antibody or antigen-binding fragment thereof) may be used to treat cardiac dysfunction in patients with a confirmed SARS-COV-2 viral infection and one or more symptoms of COVID-19, such as fever, cough, or shortness of breath. Patients with COVID-19 and pre-existing cardiovascular disease have been reported to have an increased risk of severe disease and death. In addition, SARS-COV-2 infection has been associated with multiple direct and indirect cardiovascular complications including acute myocardial injury, myocarditis, arrhythmias, and venous thromboembolism (Driggin et al., J Am Coll Cardiol., 75 (18): 2352-2371 May 2020). Hyperinflammatory responses associated with production of large amounts of pro-inflammatory cytokines and chemokines have also been reported in COVID-19 patients (Soy et al., Clinical Rheumatology, doi.org/10.1007/s10067-020-05190-5, May 2020). Without intending to be bound by theory, increases in cytokine production may activate increased Activin A expression, which in turn reduces cardiomyocyte contractile amplitude, slows contractile kinetics, and impairs cardiomyocyte calcium handling, resulting in cardiac dysfunction and, in some cases, heart failure.

There are a wide variety of approved drugs and supportive therapies currently in use to manage patients with heart failure as well as patients at risk for heart failure (e.g., patients with hypertension, a lipid disorder, diabetes, and vascular disorders). Such drugs include, for example, adrenergic blockers (alpha-and beta-blockers), centrally acting alpha-agonists, angiotensin-converting enzyme (ACE) inhibitors, angiotensin receptor blockers, calcium channel blockers, positive inotropes, vasodilators, benzodiazepines, renin inhibitors, antithrombotic agents, and multiple types of diuretics (e.g., loop, potassium-sparing, thiazide and thiazide-like). Surgical procedures for treating or preventing heart failure include, for example, physical manipulation in an attempt to increase the internal size of constricted arteries by balloon angioplasty or stenting. In some embodiments, the present disclosure provides methods of treating heart failure or one or more complications of heart failure comprising administration an Activin A specific antagonist (e.g., an anti-Activin A antibody or antigen-binding fragment thereof) in combination with an additional active agent or supportive therapy for treating, preventing or reducing the progression of heart failure (e.g., adrenergic blockers, centrally acting alpha-agonists, ACE inhibitors, angiotensin II receptor blockers, calcium channel blockers, positive inotropes, diuretics, and various surgical procedures).

### Activin A-Specific Antagonists

The methods of the present invention utilize Activin A-specific antagonists, including Activin A-specific binding proteins, small molecules inhibitors of Activin A, or nucleotide antagonists of Activin A.

As used herein, the expression “antigen-specific binding protein” means a protein comprising at least one domain which specifically binds a particular antigen. Exemplary categories of antigen-specific binding proteins include antibodies, antigen-binding portions of antibodies, peptides that specifically interact with a particular antigen (e.g., peptibodies), receptor molecules that specifically interact with a particular antigen (but not other antigens), and proteins comprising a ligand-binding portion of a receptor that specifically binds a particular antigen (but not other antigens).

The methods of the present invention include use of antigen-specific binding proteins that specifically bind Activin A, i.e., “Activin A-specific binding proteins”. Activins are homo- and hetero-dimeric molecules comprising beta subunits, i.e., Inhibin βA, inhibin βB, inhibin βC, and/or inhibin βE. The βA subunit has the amino acid sequence of SEQ ID NO:226 and the βB subunit has the amino acid sequence of SEQ ID NO:228. Activin A is a homodimer of two βA subunits; Activin B is a homodimer of two βB subunits; Activin AB is a heterodimer of one βA subunit and one βB subunit; and Activin AC is a heterodimer of one βA subunit and one βC subunit. An Activin A-specific binding protein may be an antigen-specific binding protein that specifically binds the βA subunit. Since the βA subunit is found in Activin A, Activin AB, and Activin AC molecules, an “Activin A-specific binding protein” can be an antigen-specific binding protein that specifically binds Activin A as well as Activin AB and Activin AC (by virtue of its interaction with the βA subunit). Therefore, according to one embodiment of the present invention, an Activin A-specific binding protein specifically binds Activin A; or Activin A and Activin AB; or Activin A and Activin AC; or Activin A, Activin AB and Activin AC, but does not bind other ActRIIB ligands such as Activin B, GDF3, GDF8, BMP2, BMP4, BMP7, BMP9, BMP10, GDF11, Nodal, etc. Thus, in one embodiment of the invention, an Activin A-specific binding protein specifically binds to Activin A but does not bind significantly to Activin B or Activin C. In another embodiment, an Activin A-specific binding protein may also bind to Activin B (by virtue of cross-reaction with the βB subunit, i.e., InhibinβB). In another embodiment, an Activin A-specific binding protein is a binding protein that binds specifically to Activin A but does not bind to any other ligand of ActRIIB. In another embodiment, an Activin A-specific binding protein is a binding protein and binds specifically to Activin A and does not bind to any Bone Morphogenetic Protein (BMP) (e.g., BMP2, BMP4, BMP6, BMP9, BMP10). In another embodiment, an Activin A-specific binding protein is a binding protein that binds specifically to Activin A but does not bind to any other member of the transforming growth factor beta (TGFβ) superfamily.

Some embodiments of the methods of the present invention also include antigen-specific binding proteins that specifically bind GDF8, i.e., “GDF8-specific binding proteins”. The term “GDF8” (also referred to as “growth and differentiation factor-8” and “myostatin”) means the protein having the amino acid sequence of SEQ ID NO:225 (mature protein). According to these embodiments, GDF8-specific binding proteins specifically bind GDF8 but do not bind other ActRIIB ligands such as GDF3, BMP2, BMP4, BMP7, BMP9, BMP10, GDF11, Activin A, Activin B, Activin AB, Nodal, etc.

In the context of the present invention, molecules such as ActRIIB-Fc (e.g., “ACE-031” or “RAP-031), which comprise the ligand-binding portion of the ActRIIB receptor, are not considered “Activin A-specific binding proteins” or “GDF8-specific binding proteins” because such molecules bind multiple ligands in addition to GDF8, Activin A and Activin AB.

All references to proteins, polypeptides and protein fragments herein are intended to refer to the human version of the respective protein, polypeptide or protein fragment unless explicitly specified as being from a non-human species.

### Specific Binding

The term “specifically binds” or the like, as used herein, means that an antigen-specific binding protein, or an antigen-specific binding domain, forms a complex with a particular antigen characterized by a dissociation constant (K_{D}) of 500 pM or less, and does not bind other unrelated antigens under ordinary test conditions. “Unrelated antigens” are proteins, peptides or polypeptides that have less than 95% amino acid identity to one another. Methods for determining whether two molecules specifically bind one another are well known in the art and include, for example, equilibrium dialysis, surface plasmon resonance, and the like. For example, an antigen-specific binding protein or an antigen-specific binding domain, as used in the context of the present invention, includes molecules that bind a particular antigen (e.g., Activin A and/or AB, or GDF8) or a portion thereof with a K_{D} of less than about 500 pM, less than about 400 pM, less than about 300pM, less than about 200 pM, less than about 100 pM, less than about 90 pM, less than about 80pM, less than about 70 pM, less than about 60 pM, less than about 50 pM, less than about 40 pM, less than about 30 pM, less than about 20 pM, less than about 10 pM, less than about 5 pM, less than about 4 pM, less than about 2 pM, less than about 1 pM, less than about 0.5 pM, less than about 0.2 pM, less than about 0.1 pM, or less than about 0.05 pM, as measured in a surface plasmon resonance assay.

As used herein, an antigen-specific binding protein or antigen-specific binding domain “does not bind” to a specified molecule (e.g., “does not bind GDF11”, “does not bind BMP9”, “does not bind BMP10”, etc.) if the protein or binding domain, when tested for binding to the molecule at 25° C. in a surface plasmon resonance assay, exhibits a K_{D} of greater than 50.0 nM, or fails to exhibit any binding in such an assay or equivalent thereof.

The term “surface plasmon resonance”, as used herein, refers to an optical phenomenon that allows for the analysis of real-time interactions by detection of alterations in protein concentrations within a biosensor matrix, for example using the BIAcore™ system (Biacore Life Sciences division of GE Healthcare, Piscataway, NJ).

The term “K_{D}”, as used herein, means the equilibrium dissociation constant of a particular protein-protein interaction (e.g., antibody-antigen interaction). Unless indicated otherwise, the K_{D} values disclosed herein refer to K_{D} values determined by surface plasmon resonance assay at 25° C.

### Antibodies and Antigen-Binding Fragments of Antibodies

An antigen-specific binding protein can comprise or consist of an antibody or antigen-binding fragment of an antibody. Furthermore, in the case of antigen-binding molecules comprising two different antigen-specific binding domains (discussed below), one or both of the antigen-specific binding domains may comprise or consist of an antigen-binding fragment of an antibody.

As used herein, “an antibody that binds Activin” or an “anti-Activin A antibody” includes antibodies, and antigen-binding fragments thereof, that bind a soluble fragment of the Activin A protein and may also bind to an Activin BA subunit-containing Activin heterodimer.

The term “antibody”, as used herein, means any antigen-binding molecule or molecular complex comprising at least one complementarity determining region (CDR) that specifically binds to or interacts with a particular antigen (e.g., Activin A). The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the invention, the FRs of the anti-Activin A antibody (or antigen-binding portion thereof) may be identical to the human germline sequences, or may be naturally or artificially modified. An amino acid consensus sequence may be defined based on a side-by-side analysis of two or more CDRs.

The term “antibody”, as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}-C_{H}2; (iii) V_{H}-C_{H}3;(iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody of the present invention using routine techniques available in the art.

In certain embodiments of the invention, the anti-Activin A antibodies of the invention are human antibodies. The term “human antibody,” as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The antibodies of the invention may, in some embodiments, be recombinant human antibodies. The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al., Nucl Acids Res 20:6287-6295 (1992)) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. Molecular Immunology 30:105 1993)) to levels typically observed using a human IgG1 hinge. The instant invention encompasses antibodies having one or more mutations in the hinge, C_{H}2 or C_{H}3 region which may be desirable, for example, in production, to improve the yield of the desired antibody form.

The antibodies of the invention may be isolated antibodies. An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the present invention. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The present invention includes neutralizing and/or blocking anti-Activin A antibodies. A “neutralizing” or “blocking” antibody, as used herein, is intended to refer to an antibody whose binding to Activin A: (i) interferes with the interaction between Activin A and an Activin A receptor (e.g., Activin Type IIA receptor, Activin Type IIB receptor, Activin Type I receptor, etc.); (ii) interferes with the formation of Activin-Activin receptor complexes; and/or (iii) results in inhibition of at least one biological function of Activin A. The inhibition caused by an Activin A neutralizing or blocking antibody need not be complete so long as it is detectable using an appropriate assay. Exemplary assays for detecting Activin A inhibition are described in the working examples herein.

The anti-Activin A antibodies disclosed herein may comprise one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present invention includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present invention may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present invention.

The present invention also includes anti-Activin A antibodies comprising variants of any of the HCVR, LCVR, and/or CDR amino acid sequences disclosed herein having one or more conservative substitutions. For example, the present invention includes anti-Activin A antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 95%, and more preferably at least about 96%, 97%, 98% or 99% of the nucleotide bases, as measured by any well-known algorithm of sequence identity, such as FASTA, BLAST or Gap, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson, W.R., Methods Mol Biol 24:307-331 (1994), herein incorporated by reference. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al., Science 256:1443-1445 (1992), herein incorporated by reference. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (see, e.g., Pearson, W.R., Methods Mol Biol 132:185-219 (2000), herein incorporated by reference). Another preferred algorithm when comparing a sequence of the invention to a database containing a large number of sequences from different organisms is the computer program BLAST, especially BLASTP or TBLASTN, using default parameters. See, e.g., Altschul et al., J Mol Biol 215:403-410 (1990) and Altschul et al., Nucleic Acids Res 25:3389-402 (1997), each herein incorporated by reference.

The present invention provides antibodies, or antigen-binding fragments thereof comprising a heavy chain variable region (HCVR) having an amino acid sequence selected from the group consisting of SEQ ID NO: 2, 18, 34, 50, 66, 82, 98, 106, 114, 122, 130, 138, 154, 162, 170, 178, 186, 194, and 202, or a substantially similar sequence thereof having at least 90%, at least 95%, at least 98% or at least 99% sequence identity.

The present invention also provides an antibody or antigen-binding fragment of an antibody comprising a light chain variable region (LCVR) having an amino acid sequence selected from the group consisting of SEQ ID NO: 10, 26, 42, 58, 74, 90, 146, and 210, or a substantially similar sequence thereof having at least 90%, at least 95%, at least 98% or at least 99% sequence identity.

The present invention also provides an antibody or antigen-binding fragment thereof comprising a HCVR and LCVR (HCVR/LCVR) sequence pair selected from the group consisting of SEQ ID NO: 2/10, 18/26, 34/42, 50/58, 66/74, 82/90, 98/90, 106/90, 114/90, 122/90, 130/90, 138/146, 154/146, 162/146, 170/146, 178/146, 186/146, 194/146, and 202/210.

The present invention also provides an antibody or antigen-binding fragment of an antibody comprising a heavy chain CDR3 (HCDR3) domain having an amino acid sequence selected from the group consisting of SEQ ID NO: 8, 24, 40, 56, 72, 88, 104, 112, 120, 128, 136, 144, 160, 168, 176, 184, 192, 200, and 208, or a substantially similar sequence thereof having at least 90%, at least 95%, at least 98% or at least 99% sequence identity; and a light chain CDR3 (LCDR3) domain having an amino acid sequence selected from the group consisting of SEQ ID NO: 16, 32, 48, 64, 80, 96, 152, and 216, or a substantially similar sequence thereof having at least 90%, at least 95%, at least 98% or at least 99% sequence identity.

In certain embodiments, the antibody or antigen-binding portion of an antibody comprises a HCDR3/LCDR3 amino acid sequence pair selected from the group consisting of SEQ ID NO: 8/16, 24/32, 40/48, 56/64, 72/80, 88/96, 104/96, 112/96, 120/96, 128/96, 136/96, 144/152, 160/152, 168/152, 176/152, 184/152, 192/152, 200/152, and 208/216.

The present invention also provides an antibody or fragment thereof further comprising a heavy chain CDR1 (HCDR1) domain having an amino acid sequence selected from the group consisting of SEQ ID NO: 4, 20, 36, 52, 68, 84, 100, 108, 116, 124, 132, 140, 156, 164, 172, 180, 188, 196, and 204, or a substantially similar sequence thereof having at least 90%, at least 95%, at least 98% or at least 99% sequence identity; a heavy chain CDR2 (HCDR2) domain having an amino acid sequence selected from the group consisting of SEQ ID NO: 6, 22, 38, 54, 70, 86, 102, 110, 118, 126, 134, 142, 158, 166, 174, 182, 190, 198, and 206, or a substantially similar sequence thereof having at least 90%, at least 95%, at least 98% or at least 99% sequence identity; a light chain CDR1 (LCDR1) domain having an amino acid sequence selected from the group consisting of SEQ ID NO: 12, 28, 44, 60, 76, 92, 148, and 212, or a substantially similar sequence thereof having at least 90%, at least 95%, at least 98% or at least 99% sequence identity; and a light chain CDR2 (LCDR2) domain having an amino acid sequence selected from the group consisting of SEQ ID NO: 14, 30, 46, 62, 78, 94, 150, and 214, or a substantially similar sequence thereof having at least 90%, at least 95%, at least 98% or at least 99% sequence identity.

Certain non-limiting, exemplary antibodies and antigen-binding fragments of the invention comprise HCDR1-HCDR2-HCDR3-LCDR1-LCDR2-LCDR3 domains, respectively, having the amino acid sequences selected from the group consisting of: SEQ ID NOs: 4-6-8-12-14-16 (e.g. H4H10423P); 20-22-24-28-30-32 (e.g. H4H10424P); 36-38-40-44-46-48 (e.g. H4H10426P); 52-54-56-60-62-64 (e.g. H4H10429P); 68-70-72-76-78-80 (e.g. H4H10430P); 84-86-88-92-94-96 (e.g. H4H10432P2; 100-102-104-92-94-96 (e.g. H4H10433P2); 108-110-112-92-94-96 (e.g. H4H10436P2); 116-118-120-92-94-96 (e.g. H4H10437P2); 124-126-128-92-94-96 (e.g. H4H10438P2); 132-134-136-92-94-96 (e.g. H4H10440P2); 140-142-144-148-150-152 (e.g. H4H10442P2); 156-158-160-148-150-152 (H4H10445P2); 164-166-168-148-150-152 (H4H10446P2); 172-174-176-148-150-152 (H4H10447P2); 180-182-184-148-150-152 (H4H10448P2); 188-190-192-148-150-152 (H4H10452P2); 196-198-200-148-150-152 (H4H10468P2); and 204-206-208-212-214-216 (H2aM10965N). In some embodiments, the anti-Activin A antibodies comprises the CDR sequences noted above, and a HCVR and/or LCVR that is at least 90%, 95%, 96%, 97%, 98%, or 99% identical to the corresponding HCVR and LCVR (HCVR/LCVR) sequence pair selected from the group consisting of SEQ ID NO: 2/10, 18/26, 34/42, 50/58, 66/74, 82/90, 98/90, 106/90, 114/90, 122/90, 130/90, 138/146, 154/146, 162/146, 170/146, 178/146, 186/146, 194/146, and 202/210.

In some embodiments, the invention includes an antibody or antigen-binding fragment of an antibody which specifically binds Activin A, wherein the antibody or fragment comprises the heavy and light chain CDR domains contained within heavy and light chain variable region (HCVR/LCVR) sequences selected from the group consisting of SEQ ID NO: 2/10, 18/26, 34/42, 50/58, 66/74, 82/90, 98/90, 106/90, 114/90, 122/90, 130/90, 138/146, 154/146, 162/146,170/146, 178/146, 186/146,194/146, and 202/210. Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J Mol Biol 273:927-948 (1997); and Martin et al., PNAS (USA) 86:9268-9272 (1989). Public databases are also available for identifying CDR sequences within an antibody.

The present invention includes anti-Activin A antibodies having a modified carbohydrate content. In some applications, modification to remove undesirable glycosylation sites may be useful. In some applications, modification to alter glycosylation patterns may be useful, e.g., modifying an antibody to lack a fucose moiety present on an oligosaccharide chain, for example, to increase antibody dependent cellular cytotoxicity (ADCC) function (see Shield et al. J Biol Chem 277:26733 (2002)). In other applications, modification of galactosylation can be made in order to modify complement dependent cytotoxicity (CDC). In some applications, antibodies may have modified glycosylation patterns in order to minimize effector function. For example, antibodies may be modified to obtain additionally glycosylated or sialylated antibodies.

### Biological Characteristics of the Antibodies

The present invention includes anti-Activin A antibodies and antigen-binding fragments thereof that bind Activin A with high affinity. For example, the present invention includes antibodies and antigen-binding fragments of antibodies that bind Activin A (e.g., at 25° C. or 37° C.) with a K_{D} of less than about 30 nM as measured by surface plasmon resonance, e.g., using the assay format as defined in the examples herein. In certain embodiments, the antibodies or antigen-binding fragments of the present invention bind Activin A with a K_{D} of less than about 25 nM, less than about 20 nM, less than about 15 nM, less than about 10 nM, less than about 5 nM, less than about 2 nM, less than about 1 nM, less than about 500 pM, less than about 250 pM, less than about 240 pM, less than about 230 pM, less than about 220 pM, less than about 210 pM, less than about 200 pM, less than about 190 pM, less than about 180 pM, less than about 170 pM, less than about 160 pM, less than about 150 pM, less than about 140 pM, less than about 130 pM, less than about 120 pM, less than about 110 pM, less than about 100 pM, less than about 95 pM, less than about 90 pM, less than about 85 pM, less than about 80 pM, less than about 75 pM, less than about 70 pM, less than about 65 pM, less than about 60 pM, less than about 55 pM, less than about 50 pM, less than about 45 pM, less than about 40 pM, less than about 35 pM, less than about 30 pM, less than about 25 pM, less than about 20 pM, less than about 15 pM, less than about 10 pM, less than about 9 pM, less than about 8 pM, less than about 7 pM, less than about 6 pM, less than about 5 pM, less than about 4 pM, or less than about 3 pM, as measured by surface plasmon resonance, e.g., using the assay format as defined in Example 3 herein, or a substantially similar assay.

The present invention also includes anti-Activin A antibodies and antigen-binding fragments thereof that inhibit Activin A-mediated cellular signaling. For example, the present invention includes anti-Activin A antibodies that inhibit the activation of the SMAD complex signal transduction pathway via the binding of Activin A to Activin Type I or Il receptors with an IC₅₀ value of less than about 4 nM, as measured in a cell-based blocking bioassay, e.g., using the assay format as defined in the examples herein, or a substantially similar assay. In certain embodiments, the antibodies or antigen-binding fragments of the present invention inhibit the activation of the SMAD complex signal transduction pathway via the binding of Activin A to Activin Type I or II receptors with an IC₅₀ value of less than about 3 nM, less than about 2 nM, less than about 1 nm, less than about 500 pM, less than about 250 pM, less than about 240 pM, less than about 230 pM, less than about 220 pM, less than about 210 pM, less than about 200 pM, less than about 190 pM, less than about 180 pM, less than about 170 pM, less than about 160 pM, less than about 150 pM, less than about 140 pM, less than about 130 pM, less than about 120 pM, less than about 110 pM, less than about 100 pM, less than about 95 pM, less than about 90 pM, less than about 85 pM, less than about 80 pM, less than about 75 pM, less than 70 pM, less than about 65 pM, less than about 60 pM, less than about 55 pM, less than about 50 pM, less than about 49 pM, less than about 48 pM, less than about 47 pM, less than about 46 PM, less than about 45 pM, less than about 44 pM, less than about 43 pM, less than about 42 pM, less than about 41 pM, less than about 40 pM, or less than about 39 pM, as measured in a cell-based blocking bioassay, e.g., using the assay format as defined in the examples herein, or a substantially similar assay. In certain embodiments, the antibodies or antigen-binding fragments of the present invention inhibit the signaling activing of Activin B by interfering with the binding of Activin B to Activin Type I or Il receptors with an IC₅₀ value of less than about 50 nM, less than about 20 nM, less than about 10 nm, less than about 5 nM, or less than about 1 nM,, as measured in a cell-based blocking bioassay, e.g., using the assay format as defined in the examples herein, or a substantially similar assay. In certain embodiments, the antibodies or antigen-binding fragments of the present invention inhibit the activation of the SMAD complex signal transduction pathway via the binding of Activin AB to Activin Type I or II receptors with an IC50 value of less than about 500 pM, less than about 450 pM, less than about 440 pM, less than about 430 pM, less than about 420 pM, less than about 410 pM, less than about 400 pM, less than about 390 pM, less than about 380 pM, less than about 370 pM, less than about 360 pM, less than about 350 pM, less than about 340 pM, less than about 320 pM, less than about 310 pM, less than about 300 pM, less than about 290 pM, less than about 280 pM, less than about 270 pM, less than about 260 pM, less than about 250 pM, less than about 240 pM, less than about 230 pM, less than about 220 pM, less than about 210 pM, less than about 200 pM, less than about 190 pM, less than about 180 pM, less than about 170 pM, less than about 160 pM, less than about 150 pM, or less than about 140 pM, as measured in a cell-based blocking bioassay, e.g., using the assay format as defined in the examples herein, or a substantially similar assay. In certain embodiments, the antibodies or antigen-binding fragments of the present invention inhibit the activation of the SMAD complex signal transduction pathway via the binding of Activin AC to Activin Type I or II receptors with an IC₅₀ value of less than about 1 nM, less than about 900 pM, less than about 800 pM, less than about 750 pM, less than about 700 pM, less than about 650 pM, less than about 600 pM, or less than about 580 pM, as measured in a cell-based blocking bioassay, e.g., using the assay format as defined in the examples herein, or a substantially similar assay.

The antibodies of the present invention may possess one or more of the aforementioned biological characteristics, or any combinations thereof. Other biological characteristics of the antibodies of the present invention will be evident to a person of ordinary skill in the art from a review of the present disclosure including the working examples herein.

### Anti-Activin A Antibodies Comprising Fc Variants

According to certain embodiments of the present invention, anti-Activin A antibodies are provided comprising an Fc domain comprising one or more mutations which enhance or diminish antibody binding to the FcRn receptor, e.g., at acidic pH as compared to neutral pH. For example, the present invention includes anti-Activin A antibodies comprising a mutation in the C_{H}2 or a C_{H}3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., H/L/R/S/P/Q or K) and/or 434 (e.g., A, W, H, F or Y [N434A, N434W, N434H, N434F or N434Y]); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 2591 (e.g., V259I), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P). In yet another embodiment, the modification comprises a 265A (e.g., D265A) and/or a 297A (e.g., N297A) modification.

For example, the present invention includes anti-Activin A antibodies comprising an Fc domain comprising one or more pairs or groups of mutations selected from the group consisting of: 250Q and 248L (e.g., T250Q and M248L); 252Y, 254T and 256E (e.g., M252Y, S254T and T256E); 428L and 434S (e.g., M428L and N434S); 257I and 311I (e.g., P257I and Q311I); 257I and 434H (e.g., P257I and N434H); 376V and 434H (e.g., D376V and N434H); 307A, 380A and 434A (e.g., T307A, E380A and N434A); and 433K and 434F (e.g., H433K and N434F). All possible combinations of the foregoing Fc domain mutations, and other mutations within the antibody variable domains disclosed herein, are contemplated within the scope of the present invention.

The present invention also includes anti-Activin A antibodies comprising a chimeric heavy chain constant (CH) region, wherein the chimeric CH region comprises segments derived from the CH regions of more than one immunoglobulin isotype. For example, the antibodies of the invention may comprise a chimeric CH region comprising part or all of a CH2 domain derived from a human IgG1, human IgG2 or human IgG4 molecule, combined with part or all of a CH3 domain derived from a human IgG1, human IgG2 or human IgG4 molecule. According to certain embodiments, the antibodies of the invention comprise a chimeric CH region having a chimeric hinge region. For example, a chimeric hinge may comprise an “upper hinge” amino acid sequence (amino acid residues from positions 216 to 227 according to EU numbering) derived from a human IgG1, a human IgG2 or a human IgG4 hinge region, combined with a “lower hinge” sequence (amino acid residues from positions 228 to 236 according to EU numbering) derived from a human IgG1, a human IgG2 or a human IgG4 hinge region. According to certain embodiments, the chimeric hinge region comprises amino acid residues derived from a human IgG1 or a human IgG4 upper hinge and amino acid residues derived from a human IgG2 lower hinge. An antibody comprising a chimeric CH region as described herein may, in certain embodiments, exhibit modified Fc effector functions without adversely affecting the therapeutic or pharmacokinetic properties of the antibody. (See, e.g., U.S. Provisional Appl. No. 61/759,578, filed Feb. 1, 2013, the disclosure of which is hereby incorporated by reference in its entirety).

### Epitope Mapping and Related Technologies

The present invention includes anti-Activin A antibodies which interact with one or more amino acids found within Activin A (e.g., within the Activin Type Il receptor binding site). The epitope to which the antibodies bind may consist of a single contiguous sequence of 3 or more (e.g., 3, 4, 5, 6, 7, 8, 9, 10, 11, 12, 13, 14, 15, 16, 17, 18, 19, 20 or more) amino acids located within the Activin BA subunit. Alternatively, the epitope may consist of a plurality of non-contiguous amino acids (or amino acid sequences) located within the Activin A dimer.

Various techniques known to persons of ordinary skill in the art can be used to determine whether an antibody “interacts with one or more amino acids” within a polypeptide or protein. Exemplary techniques include, e.g., routine cross-blocking assay such as that described Antibodies, Harlow and Lane (Cold Spring Harbor Press, Cold Spring Harb., NY), alanine scanning mutational analysis, peptide blots analysis (Reineke, Methods Mol Biol 248:443-463 (2004)), and peptide cleavage analysis. In addition, methods such as epitope excision, epitope extraction and chemical modification of antigens can be employed (Tomer, Protein Science 9:487-496 (2000)). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water to allow hydrogen-deuterium exchange to occur at all residues except for the residues protected by the antibody (which remain deuterium-labeled). After dissociation of the antibody, the target protein is subjected to protease cleavage and mass spectrometry analysis, thereby revealing the deuterium-labeled residues which correspond to the specific amino acids with which the antibody interacts. See, e.g., Ehring, Analytical Biochemistry 267 (2): 252-259 (1999); Engen and Smith, Anal. Chem. 73: 256A-265A (2001).

The present invention further includes anti-Activin A antibodies that bind to the same epitope as any of the specific exemplary antibodies described herein (e.g., H4H10423P, H4H10424P, H4H10426P, H4H10429P, H4H10430P, H4H10432P2, H4H10433P2, H4H10436P2, H4H10437P2, H4H10438P2, H4H10440P2, H4H10442P2, H4H10445P2, H4H10446P2, H4H10447P2, H4H10448P2, H4H10452P2, H4H10468P2, H2aM10965N, etc.). Likewise, the present invention also includes anti-Activin A antibodies that compete for binding to Activin A with any of the specific exemplary antibodies described herein (e.g., H4H10423P, H4H10424P, H4H10426P, H4H10429P, H4H10430P, H4H10432P2, H4H10433P2, H4H10436P2, H4H10437P2, H4H10438P2, H4H10440P2, H4H10442P2, H4H10445P2, H4H10446P2, H4H10447P2, H4H10448P2, H4H10452P2, H4H10468P2, H2aM10965N, etc.). For example, the present invention includes anti-Activin A antibodies that cross-compete for binding to Activin A with one or more antibodies, such as e.g., H4H10423P, H4H10446P2, H4H10468P2 and H4H10442P2. The present invention also includes anti-Activin A antibodies that cross-compete for binding to Activin A with one or more antibodies, such as e.g., H4H10429, H4H1430P, H4H10432P2, H4H10436P2, and H4H10440P2.

One can easily determine whether an antibody binds to the same epitope as, or competes for binding with, a reference anti-Activin A antibody by using routine methods known in the art and exemplified herein. For example, to determine if a test antibody binds to the same epitope as a reference anti-Activin A antibody of the invention, the reference antibody is allowed to bind to Activin A (or a βA subunit-containing heterodimer). Next, the ability of a test antibody to bind to Activin A is assessed. If the test antibody is able to bind to Activin A following saturation binding with the reference anti-Activin A antibody, it can be concluded that the test antibody binds to a different epitope than the reference anti-Activin A antibody. On the other hand, if the test antibody is not able to bind to Activin A following saturation binding with the reference anti-Activin A antibody, then the test antibody may bind to the same epitope as the epitope bound by the reference anti-Activin A antibody of the invention. Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, Biacore, flow cytometry or any other quantitative or qualitative antibody-binding assay available in the art. In accordance with certain embodiments of the present invention, two antibodies bind to the same (or overlapping) epitope if, e.g., a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. 50:1495-1502 (1990)). Alternatively, two antibodies are deemed to bind to the same epitope if essentially all amino acid mutations in the antigen that reduce or eliminate binding of one antibody reduce or eliminate binding of the other. Two antibodies are deemed to have “overlapping epitopes” if only a subset of the amino acid mutations that reduce or eliminate binding of one antibody reduce or eliminate binding of the other.

To determine if an antibody competes for binding (or cross-competes for binding) with a reference anti-Activin A antibody, the above-described binding methodology is performed in two orientations: In a first orientation, the reference antibody is allowed to bind to Activin A protein (or a βA subunit-containing heterodimer) under saturating conditions followed by assessment of binding of the test antibody to the Activin A molecule. In a second orientation, the test antibody is allowed to bind to Activin A under saturating conditions followed by assessment of binding of the reference antibody to Activin A. If, in both orientations, only the first (saturating) antibody is capable of binding to Activin A, then it is concluded that the test antibody and the reference antibody compete for binding to Activin A. As will be appreciated by a person of ordinary skill in the art, an antibody that competes for binding with a reference antibody may not necessarily bind to the same epitope as the reference antibody, but may sterically block binding of the reference antibody by binding an overlapping or adjacent epitope.

Anti-Activin A antibodies of the invention may bind to an epitope on Activin A that is within or near the binding site for an Activin Type II receptor, directly block interaction between Activin A and an Activin Type II receptor, and indirectly block interaction between Activin A and an Activin Type I receptor. Anti-Activin A antibodies of the invention may bind to an epitope on Activin A that is within or near the binding site for the Activin Type I receptor and directly block interaction between Activin A and an Activin Type I receptor. In one embodiment of the invention, an anti-Activin A antibody of the invention that binds to Activin A at or near the Activin Type I receptor binding site does not block interaction between Activin A and an Activin A Type Il receptor.

### Preparation of Human Antibodies

Methods for generating monoclonal antibodies, including fully human monoclonal antibodies are known in the art. Any such known methods can be used in the context of the present invention to make human antibodies that specifically bind to human Activin A.

Using VELOCIMMUNE™ technology, for example, or any other known method for generating fully human monoclonal antibodies, high affinity chimeric antibodies to human Activin A are initially isolated having a human variable region and a mouse constant region. As in the experimental section below, the antibodies are characterized and selected for desirable characteristics, including affinity, selectivity, epitope, etc. If necessary, mouse constant regions are replaced with a desired human constant region, for example wild-type or modified IgG1 or IgG4, to generate a fully human anti-Activin A antibody. While the constant region selected may vary according to specific use, high affinity antigen-binding and target specificity characteristics reside in the variable region. In certain instances, fully human anti-Activin A antibodies are isolated directly from antigen-positive B cells.

### Bioequivalents

The anti-Activin A antibodies and antibody fragments of the present invention encompass proteins having amino acid sequences that vary from those of the described antibodies but that retain the ability to bind human Activin A. Such variant antibodies and antibody fragments comprise one or more additions, deletions, or substitutions of amino acids when compared to parent sequence, but exhibit biological activity that is essentially equivalent to that of the described antibodies. Likewise, the anti-Activin A antibody-encoding DNA sequences of the present invention encompass sequences that comprise one or more additions, deletions, or substitutions of nucleotides when compared to the disclosed sequence, but that encode an anti-Activin A antibody or antibody fragment that is essentially bioequivalent to an anti-Activin A antibody or antibody fragment of the invention. Examples of such variant amino acid and DNA sequences are discussed above.

Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, and potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of anti-Activin A antibodies of the invention may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antibodies may include anti-Activin A antibody variants comprising amino acid changes which modify the glycosylation characteristics of the antibodies, e.g., mutations which eliminate or remove glycosylation.

### Species Selectivity and Species Cross-Reactivity

The present invention, according to certain embodiments, provides anti-Activin A antibodies that bind to human Activin A but not to Activin A from other species. The present invention also includes anti-Activin A antibodies that bind to human Activin A and to Activin A from one or more non-human species. For example, the anti-Activin A antibodies of the invention may bind to human Activin A and may bind or not bind, as the case may be, to one or more of mouse, rat, guinea pig, hamster, gerbil, pig, cat, dog, rabbit, goat, sheep, cow, horse, camel, cynomologous, marmoset, rhesus or chimpanzee Activin A. According to certain exemplary embodiments of the present invention, anti-Activin A antibodies are provided which specifically bind human Activin A (e.g., Activin A or a βA subunit-containing heterodimer) and cynomolgus monkey (e.g., Macaca fascicularis) Activin A.

### Multispecific Antibodies

The antibodies of the present invention may be monospecific, bi-specific, or multispecific. Multispecific antibodies may be specific for different epitopes of one target polypeptide or may contain antigen-binding domains specific for more than one target polypeptide. See, e.g., Tutt et al., J Immunol 147:60-69 (1991); Kufer et al., Trends Biotechnol 22:238-244 (2004). The anti-Activin A antibodies of the present invention can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bi-specific or a multispecific antibody with a second binding specificity. For example, the present invention includes bi-specific antibodies wherein one arm of an immunoglobulin is specific for human Activin A or a fragment thereof, and the other arm of the immunoglobulin is specific for a second therapeutic target or is conjugated to a therapeutic moiety. One embodiment of the invention includes bi-specific antibodies wherein one arm of an immunoglobulin is specific for human Activin A or a fragment thereof, and the other arm of the immunoglobulin is specific for GDF8.

An exemplary bi-specific antibody format that can be used in the context of the present invention involves the use of a first immunoglobulin (Ig) C_{H}3 domain and a second Ig C_{H}3 domain, wherein the first and second Ig C_{H}3 domains differ from one another by at least one amino acid, and wherein at least one amino acid difference reduces binding of the bispecific antibody to Protein A as compared to a bi-specific antibody lacking the amino acid difference (see, e.g., U.S. Pat. No. 8,586,713, incorporated by reference herein in its entirety). In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). Further modifications that may be found within the second C_{H}3 include: D16E, L18M, N44S, K52N, V57M, and V82I (by IMGT; D356E, L358M, N384S, K392N, V397M, and V422I by EU) in the case of IgG1 antibodies; N44S, K52N, and V82I (IMGT; N384S, K392N, and V422I by EU) in the case of IgG2 antibodies; and Q15R, N44S, K52N, V57M, R69K, E79Q, V82I, and L105P (by IMGT; Q355R, N384S, K392N, V397M, R409K, E419Q, V422I, and L445P by EU) in the case of IgG4 antibodies. Variations on the bi-specific antibody format described above are contemplated within the scope of the present invention.

Other exemplary bispecific formats that can be used in the context of the present invention include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mab² bispecific formats (see, e.g., Klein et al., mAbs 4:6, 1-11 (2012), and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J Am Chem Soc. 135 (1): 340-346 (2013)).

### Therapeutic Formulation and Administration

The anti-Activin A antibodies (or other Activin A specific antagonists) used in the methods of the present invention may be formulated for administration in pharmaceutical compositions with one or more pharmaceutically acceptable carriers, excipients or diluents. The pharmaceutical compositions are formulated with suitable carriers, excipients, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™, Life Technologies, Carlsbad, CA), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA, J Pharm Sci Technol 52:238-311 (1998).

The dose of antibody administered to a patient may vary depending upon the age and the size of the patient, target disease, conditions, route of administration, and the like. The preferred dose is typically calculated according to body weight or body surface area. When an antibody of the present invention is used for treating a condition or disease associated with Activin A activity in an adult patient, it may be advantageous to intravenously administer the antibody of the present invention normally at a single dose of about 0.01 to about 20 mg/kg body weight, more preferably about 0.02 to about 7, about 0.03 to about 5, or about 0.05 to about 3 mg/kg body weight. In some cases, the dose is 3 mg/kg. In some cases, the does is 10 mg/kg. Depending on the severity of the condition, the frequency and the duration of the treatment can be adjusted. A patient with “severe” disease (e.g., COVID-19) requires supplemental oxygen administration by nasal canula, simple face mask, or other similar oxygen delivery device. A patient with “critical” disease (e.g., COVID-19) requires supplemental oxygen delivered by non-rebreather mask of high-flow nasal canula or the use of invasive or non-invasive ventilation or requires treatment in an intensive care unit. Effective dosages and schedules for administering anti-Activin A antibodies may be determined empirically; for example, patient progress can be monitored by periodic assessment, and the dose adjusted accordingly. Moreover, interspecies scaling of dosages can be performed using well-known methods in the art (e.g., Mordenti et al., Pharmaceut Res 8:1351 (1991)).

Various delivery systems are known and can be used to administer the pharmaceutical composition of the invention, e.g., encapsulation in liposomes, microparticles, microcapsules, recombinant cells capable of expressing an antibody or other therapeutic protein of the invention, receptor mediated endocytosis (see, e.g., Wu et al., J Biol Chem 262:4429-4432 (1987)). The antibodies and other therapeutically active components of the present invention may also be delivered by gene therapy techniques. Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

A pharmaceutical composition can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition as discussed herein. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present invention include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L.P.), and the HUMIRA™ Pen (Abbott Labs, Abbott Park IL), to name only a few.

In certain situations, the pharmaceutical composition can be delivered in a controlled release system. In one embodiment, a pump may be used (see Langer, supra; Sefton, CRC Crit. Ref. Biomed. Eng. 14:201 (1987)). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, Science 249:1527-1533 (1990).

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents.

### Combination Therapies

The present invention includes methods comprising the use or administration of any of the anti-Activin A antibodies described herein in combination with one or more additional therapeutically active components. In some cases, the anti-Activin A antibodies of the invention may also be administered and/or co-formulated in combination with antivirals, antibiotics, analgesics, corticosteroids, steroids, oxygen, antioxidants, metal chelators, IFN-gamma, and/or NSAIDs. In some cases, the anti-Activin A antibodies of the invention may also be administered or used in combination with an additional active agent or other supportive therapy for treating, preventing, or reducing the severity of heart failure or one or more complications of heart failure. In some cases, the additional active agent or other supportive therapy is selected from the group consisting of: pacemaker, implantable cardiac defibrillator, cardiac contractility modulation, cardiac resynchronization therapy, ventricular assist device, biventricular cardiac resynchronization therapy, heart transplant, adrenergic blockers (alpha-and beta-blockers), centrally acting alpha-agonists, angiotensin-converting enzyme (ACE) inhibitors, angiotensin receptor blockers, calcium channel blockers, positive inotropes, vasodilators, benzodiazepines, renin inhibitors, antithrombotic agents, multiple types of diuretics, captopril, enalapril, lisinopril, benazepril, ramipril, Zofenopril, quinapril, perinodopril, lisinopril, benazepril, imidapril, trandolapril, cilazapril, and fosinopril, losartan, candesartan, valsartan, irbesartan, telmisartan, eprosartan, olmesartan, azilsartan, Fimasartan, propranolol, bucindolol, carteolol, carvedilol, labetalol, nadolol, oxprenolol, penbutolol, pindolol, sotalol, timolol, acebutolol, atenolol, betaxolol, bisoprolol, celiprolol, esmolol, metoprolol, nebivolol, butazamine, ICI-118,551, SR 59230A, phenoxybenzamine, phentolamine, tolazoline, trazodone, alfuzosin, doxazosin mesylate (Cardura and Carduran), prazosin, tamsulosin, terazosin, Silodosin, atipanmezole (e.g., Antisedan), idazoxan, mirtazapine, yohimbine, acidifying salts (e.g., CaCl₂ and NH₄Cl), arginine vasopressin receptor 2 antagonists, selective vasopressin V2 antagonists, Na—H exchanger antagonists, carbonic anhydrase inhibitors, loop diuretics, osmotic diuretics, potassium- sparing diuretics, thiazides, xanthines, dihydropyridine, amlodipine, aranidipine, azelnidipine, barnidipine, benidipine, cilnidipine, clevidipine, isradipine, efonidipine, felodipine, lacidipine, lercanidipine, manidipine, nicardipine, nifedipine, Nilvadipine, nimodipine, nisoldipine, nitrendipine, pranidipine, phenylalkylamine calcium channel blockers, verapamil, gallopamil, fendiline, benzothiazepine calcium channel blockers, diltiazem, mibefradil, bepridil, flunarizine, fluspirilene, fendiline, gabapentinoids, ziconotide, digoxin, amiodarone, berberine, levosimendan, omecamtiv, catecholamines, eicosanoids, phosphodiesterase inhibitors, enoximone, milrinone, amrinone, theophylline, glucagon, insulin, sodium nitroprusside, hydralazine, isosorbide dinitrate, and isosorbide mononitrate, nitroglycerin, benzodiazepines, renin inhibitors, clonidine, guanabenz, guanfacine, methyldopa, and moxonidine, minoxidil, guanethidine, mecamylamine, reserpine, irreversible cyclooxygenase inhibitors, adenosine diphosphate receptor inhibitors, clopidogrel, prasugrel, ticagrelor, and ticlopidine, phosphodiesterase inhibitors, cilostazol, protease-activated receptor-1 antagonists, vorapaxar, glycoprotein inhibitors, abciximab, eptifibatide, tirofiban, adenosine reuptake inhibitors, dipyridamole, thromboxane inhibitors, thromboxane synthase inhibitors, and thromboxane receptor antagonists, tissue plasminogen activators, alteplase, reteplase, tenecteplase, anistreplase, streptokinase, urokinase, dabigatran, rivaroxaban, apixaban, coumarins, heparin and derivatives thereof, factor Xa inhibitors, rivaroxaban, apixaban, edoxaban, betrixaban, letaxaban, eribaxaban, hirudin, lepirudin, bivalirudin, argatroban, dabigatran, ximelagatran, antithrombin protein, batroxobin, hementin, and vitamin E. In some embodiments, any of the anti-Activin A antibodies of the present invention may also be administered and/or co- formulated in combination with a GDF8 inhibitor (e.g., an anti-GDF8 antibody).

The additional therapeutically active component(s) or supportive therapy may be administered to a subject or used prior to administration of an anti-Activin A antibody of the present invention. For example, a first component may be deemed to be administered/used “prior to” a second component if the first component is administered/used 1 week before, 72 hours before, 60 hours before, 48 hours before, 36 hours before, 24 hours before, 12 hours before, 6 hours before, 5 hours before, 4 hours before, 3 hours before, 2 hours before, 1 hour before, 30 minutes before, 15 minutes before, 10 minutes before, 5 minutes before, or less than 1 minute before administration/use of the second component. In other embodiments, the additional therapeutically active component(s) or supportive therapy may be administered to a subject or used after administration of an anti-Activin A antibody of the present invention. For example, a first component may be deemed to be administered/used “after” a second component if the first component is administered/used 1 minute after, 5 minutes after, 10 minutes after, 15 minutes after, 30 minutes after, 1 hour after, 2 hours after, 3 hours after, 4 hours after, 5 hours after, 6 hours after, 12 hours after, 24 hours after, 36 hours after, 48 hours after, 60 hours after, 72 hours after administration/use of the second component. In yet other embodiments, the additional therapeutically active component(s) may be administered to a subject or used concurrent with administration of anti-Activin A antibody of the present invention. “Concurrent” administration, for purposes of the present invention, includes, e.g., administration of an anti-Activin A antibody and an additional therapeutically active component to a subject in a single dosage form, or in separate dosage forms administered to the subject within about 30 minutes or less of each other. If administered in separate dosage forms, each dosage form may be administered via the same route (e.g., both the anti-Activin A antibody and the additional therapeutically active component may be administered intravenously, subcutaneously, intravitreally, etc.); alternatively, each dosage form may be administered via a different route (e.g., the anti-Activin A antibody may be administered locally (e.g., intravitreally) and the additional therapeutically active component may be administered systemically). In any event, administering the components in a single dosage from, in separate dosage forms by the same route, or in separate dosage forms by different routes are all considered “concurrent administration,” for purposes of the present disclosure. For purposes of the present disclosure, administration of an anti-Activin A antibody “prior to”, “concurrent with,” or “after” (as those terms are defined herein above) administration of an additional therapeutically active component is considered administration of an anti-Activin A antibody “in combination with” an additional therapeutically active component).

The present invention includes pharmaceutical compositions in which an anti-Activin A antibody of the present invention is co-formulated with one or more of the additional therapeutically active component(s) as described elsewhere herein.

### Dosage

The amount of active ingredient (e.g., anti-Activin A antibodies, anti-GDF8 antibodies, or other therapeutic agents given in combination with anti-Activin A antibodies, or bispecific antibodies that specifically bind Activin A and GDF8) that can be administered to a subject is, generally, a therapeutically effective amount, as discussed elsewhere herein.

In some embodiments, a therapeutically effective amount can be from about 0.05 mg to about 600 mg; e.g., about 0.05 mg, about 0.1 mg, about 1.0 mg, about 1.5 mg, about 2.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, or about 1000 mg, of the respective antibody.

The amount of anti-Activin A antibody or other therapeutic agent contained within the individual doses may be expressed in terms of milligrams of antibody per kilogram of patient body weight (i.e., mg/kg). For example, the anti-Activin A, anti-GDF8 and/or anti-Activin A/anti-GDF8 bispecific antibodies may be administered to a patient at a dose of about 0.0001 to about 50 mg/kg of patient body weight (e.g. 0.1 mg/kg, 0.5 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0 mg/kg, 6.5 mg/kg, 7.0 mg/kg, 7.5 mg/kg, 8.0 mg/kg, 8.5 mg/kg, 9.0 mg/kg, 9.5 mg/kg, 10.0 mg/kg, 10.5 mg/kg, 11.0 mg/kg, 11.5 mg/kg, 12.0 mg/kg, 12.5 mg/kg, 13.0 mg/kg, 13.5 mg/kg, 14.0 mg/kg, 14.5 mg/kg, 15.0 mg/kg, 15.5 mg/kg, 16.0 mg/kg, 16.5 mg/kg, 17.0 mg/kg, 17.5 mg/kg, 18.0 mg/kg, 18.5 mg/kg, 19.0 mg/kg, 19.5 mg/kg, 20.0 mg/kg, etc.).

### Administration Regimens

According to certain embodiments of the present invention, multiple doses of an active ingredient (e.g., an anti-Activin A antibody, an anti-GDF8 antibody administered in combination with an anti-Activin A antibody, a pharmaceutical composition comprising a combination of anti-Activin A antibody and any of the additional therapeutically active agents mentioned herein, including, e.g., an anti-GDF8 antibody, or a bispecific antibody that specifically bind Activin A and GDF8) may be administered to a subject over a defined time course. The methods according to this aspect of the invention comprise sequentially administering to a subject multiple doses of an active ingredient of the invention. As used herein, “sequentially administering” means that each dose of an active ingredient is administered to the subject at a different point in time, e.g., on different days separated by a predetermined interval (e.g., hours, days, weeks or months). The present invention includes methods which comprise sequentially administering to the patient a single initial dose of an active ingredient, followed by one or more secondary doses of the active ingredient, and optionally followed by one or more tertiary doses of the active ingredient.

The terms “initial dose,” “secondary doses,” and “tertiary doses,” refer to the temporal sequence of administration of the active ingredient, e.g., anti-Activin A antibody of the invention or of a combination therapy of the invention, e.g., an anti-Activin A antibody and an anti- GDF8 antibody. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the active ingredient, e.g., anti-Activin A antibody, but generally may differ from one another in terms of frequency of administration. In certain embodiments, however, the amount of the active ingredient, e.g., anti-Activin A antibody, contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In certain exemplary embodiments of the present invention, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose of the active ingredient, e.g., an anti- Activin A antibody, which is administered to a patient prior to the administration of the very next dose in the sequence with no intervening doses.

The methods according to this aspect of the invention may comprise administering to a patient any number of secondary and/or tertiary doses of an active ingredient of the invention, e.g., an anti-Activin A antibody. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks or 1 to 2 months after the immediately preceding dose. Similarly, in embodiments involving multiple tertiary doses, each tertiary dose may be administered at the same frequency as the other tertiary doses. For example, each tertiary dose may be administered to the patient 2 to 12 weeks after the immediately preceding dose. In certain embodiments of the invention, the frequency at which the secondary and/or tertiary doses are administered to a patient can vary over the course of the treatment regimen. The frequency of administration may also be adjusted during the course of treatment by a physician depending on the needs of the individual patient following clinical examination.

The present invention includes administration regimens in which 2 to 6 loading doses are administered to a patient a first frequency (e.g., once a week, once every two weeks, once every three weeks, once a month, once every two months, etc.), followed by administration of two or more maintenance doses to the patient on a less frequent basis. For example, according to this aspect of the invention, if the loading doses are administered at a frequency of once a month, then the maintenance doses may be administered to the patient once every six weeks, once every two months, once every three months, etc.).

### Kits

The present invention further provides an article of manufacturing or kit, comprising a packaging material, container and a pharmaceutical agent contained within the container, wherein the pharmaceutical agent comprises at least one Activin A antagonist (e.g., an anti-Activin A antibody), and wherein the packaging material comprises a label or package insert showing indications and directions for use (e.g., use of the anti-Activin A antibody to treat cardiac dysfunction or heart failure).

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1. Generation of Human Antibodies to Activin A

An immunogen comprising the Activin A protein (inhibin-βA dimer) was administered directly, with an adjuvant to stimulate the immune response, to a VELOCIMMUNE® mouse comprising DNA encoding human Immunoglobulin heavy and kappa light chain variable regions. The antibody immune response was monitored by a Activin A-specific immunoassay. When a desired immune response was achieved splenocytes were harvested and fused with mouse myeloma cells to preserve their viability and form hybridoma cell lines. The hybridoma cell lines were screened and selected to identify cell lines that produce Activin A-specific antibodies. Using this technique several anti-Activin A chimeric antibodies (i.e., antibodies possessing human variable domains and mouse constant domains) were obtained. An exemplary antibody obtained in this manner is H2aM10965N. The human variable domains from the chimeric antibodies were subsequently cloned onto human constant domains to make fully human anti-Activin A antibodies as described herein.

Anti-Activin A antibodies were also isolated directly from antigen-positive B cells without fusion to myeloma cells, as described in US 2007/0280945A1. Using this method, several fully human anti-Activin A antibodies (i.e., antibodies possessing human variable domains and human constant domains) were obtained; exemplary antibodies generated in this manner were designated as follows: H4H10423P, H4H10429P, H4H10430P, H4H10432P2, H4H10440P2, H4H10442P2, H4H10436P2, and H4H10446P2.

Certain biological properties of the exemplary anti-Activin A antibodies generated in accordance with the methods of this Example are described in detail in the Examples set forth below.

### Example 2. Heavy and Light Chain Variable Region Amino Acid Sequences

Table 1 sets forth the heavy and light chain variable region amino acid sequence pairs of selected anti-Activin A antibodies and their corresponding antibody identifiers. The corresponding nucleic acid sequence identifiers are set forth in Table 2.

TABLE 1
Amino Acid Sequence Identifiers

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| H4H10423P | 2 | 4 | 6 | 8 | 10 | 12 | 14 | 16 |
| H4H10424P | 18 | 20 | 22 | 24 | 26 | 28 | 30 | 32 |
| H4H10426P | 34 | 36 | 38 | 40 | 42 | 44 | 46 | 48 |
| H4H10429P | 50 | 52 | 54 | 56 | 58 | 60 | 62 | 64 |
| H4H10430P | 66 | 68 | 70 | 72 | 74 | 76 | 78 | 80 |
| H4H10432P2 | 82 | 84 | 86 | 88 | 90 | 92 | 94 | 96 |
| H4H10433P2 | 98 | 100 | 102 | 104 | 90 | 92 | 94 | 96 |
| H4H10436P2 | 106 | 108 | 110 | 112 | 90 | 92 | 94 | 96 |
| H4H10437P2 | 114 | 116 | 118 | 120 | 90 | 92 | 94 | 96 |
| H4H10438P2 | 122 | 124 | 126 | 128 | 90 | 92 | 94 | 96 |
| H4H10440P2 | 130 | 132 | 134 | 136 | 90 | 92 | 94 | 96 |
| H4H10442P2 | 138 | 140 | 142 | 144 | 146 | 148 | 150 | 152 |
| H4H10445P2 | 154 | 156 | 158 | 160 | 146 | 148 | 150 | 152 |
| H4H10446P2 | 162 | 164 | 166 | 168 | 146 | 148 | 150 | 152 |
| H4H10447P2 | 170 | 172 | 174 | 176 | 146 | 148 | 150 | 152 |
| H4H10448P2 | 178 | 180 | 182 | 184 | 146 | 148 | 150 | 152 |
| H4H10452P2 | 186 | 188 | 190 | 192 | 146 | 148 | 150 | 152 |
| H4H10468P2 | 194 | 196 | 198 | 200 | 146 | 148 | 150 | 152 |
| H2aM10965N | 202 | 204 | 206 | 208 | 210 | 212 | 214 | 216 |

TABLE 2
Nucleic Acid Sequence Identifiers

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| H4H10423P | 1 | 3 | 5 | 7 | 9 | 11 | 13 | 15 |
| H4H10424P | 17 | 19 | 21 | 23 | 25 | 27 | 29 | 31 |
| H4H10426P | 33 | 35 | 37 | 39 | 41 | 43 | 45 | 47 |
| H4H10429P | 49 | 51 | 53 | 55 | 57 | 59 | 61 | 63 |
| H4H10430P | 65 | 67 | 69 | 71 | 73 | 75 | 77 | 79 |
| H4H10432P2 | 81 | 83 | 85 | 87 | 89 | 91 | 93 | 95 |
| H4H10433P2 | 97 | 99 | 101 | 103 | 89 | 91 | 93 | 95 |
| H4H10436P2 | 105 | 107 | 109 | 111 | 89 | 91 | 93 | 95 |
| H4H10437P2 | 113 | 115 | 117 | 119 | 89 | 91 | 93 | 95 |
| H4H10438P2 | 121 | 123 | 125 | 127 | 89 | 91 | 93 | 95 |
| H4H10440P2 | 129 | 131 | 133 | 135 | 89 | 91 | 93 | 95 |
| H4H10442P2 | 137 | 139 | 141 | 143 | 145 | 147 | 149 | 151 |
| H4H10445P2 | 153 | 155 | 157 | 159 | 145 | 147 | 149 | 151 |
| H4H10446P2 | 161 | 163 | 165 | 167 | 145 | 147 | 149 | 151 |
| H4H10447P2 | 169 | 171 | 173 | 175 | 145 | 147 | 149 | 151 |
| H4H10448P2 | 177 | 179 | 181 | 183 | 145 | 147 | 149 | 151 |
| H4H10452P2 | 185 | 187 | 189 | 191 | 145 | 147 | 149 | 151 |
| H4H10468P2 | 193 | 195 | 197 | 199 | 145 | 147 | 149 | 151 |
| H2aM10965N | 201 | 203 | 205 | 207 | 209 | 211 | 213 | 215 |

Antibodies are typically referred to herein according to the following nomenclature: Fc prefix (e.g. “H1M,” “H2aM,” “H4H”), followed by a numerical identifier (e.g. “10423,” “10424,” or “10426” as shown in Tables 1 and 2), followed by a “P,” “P2” or “N” suffix. Thus, according to this nomenclature, an antibody may be referred to herein as, e.g., “H4H10423P,” “H4H10432P2,” “H2aM10965N,” etc. The H1M, H2M and H4H prefixes on the antibody designations used herein indicate the particular Fc region isotype of the antibody. For example, an “H2aM” antibody has a mouse lgG2a Fc, whereas an “H4H” antibody has a human lgG4 Fc. As will be appreciated by a person of ordinary skill in the art, an antibody having a particular Fc isotype can be converted to an antibody with a different Fc isotype (e.g., an antibody with a mouse IgG2a Fc can be converted to an antibody with a human IgG4, etc.), but in any event, the variable domains (including the CDRs)—which are indicated by the numerical identifiers shown in Table 1—will remain the same, and the binding properties are expected to be identical or substantially similar regardless of the nature of the Fc domain.

### Control Constructs Used in the Following Examples

Anti-Activin A control molecules were included in the following Examples for comparative purposes. The control antibody designated herein as Control 1 is a human anti-Activin A antibody with heavy and light chain variable domain sequences of “A1” as set forth in U.S. Pat. No. 8,309,082. Control 2 is an anti-human Activin Receptor Type II B antibody (anti-ActR2B mAb) disclosed as MOR8159 in US Patent Application No. 2012/0237521 A1. Control 3 is a murine anti-Activin A monoclonal antibody from R&D Systems, Minneapolis, MN (catalog number MAB3381). Control 4 is an Activin Type IIB receptor-Fc fusion molecule (a soluble Activin RIIB receptor extracelullar domain produced with a C-terminal human IgG1 Fc fusion protein (E23-P133 of NP_001097 followed by a Gly-Ser linker followed by a C-terminal human lgG1 Fc fusion), the sequence of which is provided as SEQ ID NO:227.

### Example 3. Antibody Binding to Human Activin A as Determined by Surface Plasmon Resonance

Binding affinities and kinetic constants for antigen binding to selected purified anti-human Activin A monoclonal antibodies were determined using a real-time surface plasmon resonance biosensor (Biacore T200 or Biacore 4000, GE Healthcare Life Sciences, Piscataway, NJ) assay at 25° C. and 37° C. Antibodies, expressed as either mouse Fc (prefix H2aM) or human Fc (prefix H4H), were captured on their respective anti-Fc sensor surfaces (mAb capture format). Anti-Activin A antibodies were captured on either a goat anti-mouse IgG polyclonal antibody (GE Healthcare, #BR-1008-38) or a mouse anti-human IgG monoclonal antibody (GE Healthcare, #BR-1008-39) surface created through direct amine coupling to a Biacore CM5 sensor chip. Kinetic experiments were carried out using either HBS-EP (10 mM HEPES, 150 mM NaCl, 3 mM EDTA, 0.05% Surfactant P20, at pH 7.4) or PBS-P (10 mM Sodium Phosphate, 2.7 mM KCl, 137 mM NaCl, 0.02% NaN3, 0.05% Surfactant P20, pH 7.4), as both the running buffer and the sample buffer. Antigen-antibody association rates were measured by injecting various concentrations (4-fold dilutions ranging from 50 to 0.2 nM) of either Activin A (R&D Systems, #338-AC-050/CF), Activin B (R&D Systems, #659-AB-025/CF), Activin AB (R&D Systems, #1006-AB-005), Activin AC (R&D Systems, #4879-AC/CF), or Inhibin E (Novus Biologicals, #H00083729-P01) over the captured antibody surface. Antibody-antigen association was monitored for 240 seconds while dissociation in buffer was monitored for 600 seconds. Kinetic association and dissociation rate constants were determined by processing and fitting the data using Scrubber software version 2.0c. Binding equilibrium dissociation constants (K_{D}) and dissociative half-lives (t_{1/2}) were then calculated from the kinetic rate constants as: K_{D}(M)=K_{d}/Kₐ and t_{1/2} (min)=[ln2/(60*K_{d})]. Kinetic binding parameters for different anti-Activin A monoclonal antibodies are shown in Tables 3 to 10. (NB=no binding observed under the conditions used; NT=not tested).

TABLE 3
Binding Characteristics of Anti-Activin A Antibodies to Activin A at 25° C.

| | Amount of | Activin | | | | |
|---|---|---|---|---|---|---|
| | mAb | A- | | | | |
| | Captured | 20 nM | | | | |
| Antibody | (RU ± SE) | (RU) | kₐ (M⁻¹s⁻¹) | k_{d} (s⁻¹) | K_{D} (Molar) | t_{1/2} (min) |
| H4H10423P | 86.2 ± 0.7 | 19.4 | 3.33E+06 | 1.09E−04 | 3.26E−11 | 106.4 |
| H4H10424P | 337 | 82 | 3.14E+06 | 7.19E−04 | 2.29E−10 | 16 |
| H4H10426P | 81 | 23 | 1.18E+07 | 7.00E−04 | 5.95E−11 | 16 |
| H4H10429P | 115.2 ± 1 | 24.9 | 7.82E+06 | 6.39E−05 | 8.17E−12 | 180.8 |
| H4H10430P | 90.3 ± 4.2 | 19.4 | 4.75E+07 | 1.67E−04 | 3.52E−12 | 69 |
| H4H10432P2 | 109.6 ± 1.2 | 20.7 | 1.57E+07 | 5.00E−05 | ≤3.18E−12 | ≥231 |
| H4H10433P2 | 102 | 16 | 1.42E+07 | 5.77E−04 | 4.06E−11 | 20 |
| H4H10436P2 | 113.6 ± 0.6 | 23.2 | 8.85E+06 | 1.68E−04 | 1.90E−11 | 68.7 |
| H4H10437P2 | 167 | 30 | 1.58E+07 | 2.13E−03 | 1.34E−10 | 5 |
| H4H10438P2 | 124 | 25 | 1.20E+07 | 5.88E−04 | 4.92E−11 | 20 |
| H4H10440P2 | 79.2 ± 0.7 | 12.9 | 3.76E+06 | 9.28E−05 | 2.47E−11 | 124.5 |
| H4H10442P2 | 139.3 ± 1 | 31.3 | 1.10E+07 | 5.00E−05 | ≤4.55E−12 | ≥231 |
| H4H10445P2 | 149 | 43 | 2.40E+06 | 5.00E−05 | ≤2.08E−11 | ≥231 |
| H4H10446P2 | 104.6 ± 0.7 | 24.1 | 1.29E+07 | 5.00E−05 | ≤3.88E−12 | ≥231 |
| H4H10447P2 | 164 | 43 | 2.36E+06 | 5.00E−05 | ≤2.12E−11 | ≥231 |
| H4H10448P2 | 244 | 64 | 4.76E+06 | 5.00E−05 | ≤1.05E−11 | ≥231 |
| H4H10452P2 | 191 | 55 | 4.69E+06 | 5.00E−05 | ≤1.07E−11 | ≥231 |
| H4H10468P2 | 93 ± 0.1 | 21.7 | 7.86E+06 | 5.00E−05 | ≤6.36E−12 | ≥231 |
| H2aM10965N | 393 | 76 | 1.48E+06 | 1.10E−03 | 7.45E−10 | 10 |
| Control 1 | 84.7 ± 0.3 | 15.9 | 7.26E+06 | 9.92E−05 | 1.37E−11 | 116.4 |

For k_{d} values that are italicized, no dissociation of the analyte was observed under these experimental conditions, and the value of k_{d} was therefore fixed at 5.0E-05 s⁻¹

TABLE 4
Binding Characteristics of Anti-Activin A Antibodies to to Activin A at 37° C.

| | Amount of | Activin | | | | |
|---|---|---|---|---|---|---|
| | mAb | A- | | | | |
| | Captured | 20 nM | | | | t_{1/2} |
| Antibody | (RU ± SE) | (RU) | kₐ (M⁻¹s⁻¹) | kd (s⁻¹) | K_{D} (Molar) | (min) |
| H4H10423P | 101 ± 1.4 | 25.2 | 3.95E+06 | 5.00E−05 | ≤1.26E−11 | ≥231 |
| H4H10424P | 231 | 58 | 4.59E+06 | 3.64E−03 | 7.94E−10 | 3 |
| H4H10426P | 71 | 21 | 1.61E+07 | 1.98E−03 | 1.23E−10 | 6 |
| H4H10429P | 150.8 ± 5.3 | 31.4 | 1.33E+07 | 5.00E−05 | ≤3.75E−12 | ≥231 |
| H4H10430P | 109.3 ± 1.3 | 25.0 | 3.80E+07 | 1.51E−04 | 3.97E−12 | 76.5 |
| H4H10432P2 | 141.8 ± 1.6 | 25.1 | 2.30E+07 | 5.00E−05 | ≤2.18E−12 | ≥231 |
| H4H10433P2 | 85 | 12 | 2.00E+07 | 1.07E−03 | 5.37E−11 | 11 |
| H4H10436P2 | 139.8 ± 1.4 | 29.4 | 1.49E+07 | 5.00E−05 | ≤3.35E−12 | ≥231 |
| H4H10437P2 | 115 | 20 | 2.04E+07 | 4.68E−03 | 2.29E−10 | 2 |
| H4H10438P2 | 99 | 18 | 1.87E+07 | 2.38E−03 | 1.27E−10 | 5 |
| H4H10440P2 | 98.6 ± 1.1 | 15.3 | 6.37E+06 | 3.28E−04 | 5.15E−11 | 35.2 |
| H4H10442P2 | 181 ± 2.5 | 40.5 | 1.44E+07 | 5.00E−05 | ≤3.48E−12 | ≥231 |
| H4H10445P2 | 120 | 36 | 4.33E+06 | 5.00E−05 | ≤1.15E−11 | ≥231 |
| H4H10446P2 | 137.2 ± 1.7 | 31.5 | 1.54E+07 | 5.00E−05 | ≤3.25E−12 | ≥231 |
| H4H10447P2 | 126 | 36 | 4.69E+06 | 5.00E−05 | ≤1.07E−11 | ≥231 |
| H4H10448P2 | 175 | 49 | 7.86E+06 | 5.00E−05 | ≤6.36E−12 | ≥231 |
| H4H10452P2 | 146 | 43 | 7.94E+06 | 5.00E−05 | ≤6.30E−12 | ≥231 |
| H4H10468P2 | 98.7 ± 0.7 | 24.5 | 1.22E+07 | 5.00E−05 | ≤4.10E−12 | ≥231 |
| H2aM10965N | 435 | 80 | 2.35E+06 | 4.15E−03 | 1.77E−09 | 3 |
| Control 1 | 93.9 ± 0.7 | 18.0 | 8.99E+06 | 5.00E−05 | ≤5.56E−12 | ≥231 |

For k_{d} values that are italicized, no dissociation of the analyte was observed under these experimental conditions, and the value of k_{d} was therefore fixed at 5.0E-05 s⁻¹

TABLE 5
Binding Characteristics of Anti-Activin A Antibodies to Activin B at 25° C.

| | Amount of | 50 nM | | | | |
|---|---|---|---|---|---|---|
| | mAb | Ag | | | | |
| | Captured | Bound | | | | |
| Antibody | (RU ± SE) | (RU) | kₐ (M⁻¹s⁻¹) | k_{d} (s⁻¹) | K_{D} (Molar) | t_{1/2} (min) |
| H4H10423P | 83.1 ± 0.6 | 4.7 | 4.89E+05 | 3.02E−02 | 6.18E−08 | 0.4 |
| H4H10429P | 112.3 ± 0.7 | 26.4 | 3.49E+06 | 1.31E−02 | 3.75E−09 | 0.9 |
| H4H10432P2 | 104.4 ± 1.8 | 5.1 | NB | NB | NB | NB |
| H4H10436P2 | 110.8 ± 3.9 | 32.8 | 9.52E+06 | 5.28E−04 | 5.54E−11 | 21.9 |
| H4H10440P2 | 75.7 ± 0.8 | 18.8 | 1.06E+06 | 1.16E−03 | 1.09E−09 | 10.0 |
| H4H10442P2 | 136 ± 0.7 | 3.4 | NB | NB | NB | NB |
| H4H10430P | 88 ± 0.5 | 3.9 | NB | NB | NB | NB |
| H4H10446P2 | 101.5 ± 0.4 | 3.6 | NB | NB | NB | NB |
| H4H10468P2 | 92.5 ± 0.2 | 6.2 | NB | NB | NB | NB |
| Control 1 | 84.1 ± 0.3 | 6.4 | NB | NB | NB | NB |

TABLE 6
Binding Characteristics of Anti-Activin A Antibodies to Activin B at 37° C.

| | Amount of | 50 nM | | | | |
|---|---|---|---|---|---|---|
| | mAb | Ag | | | | |
| | Captured | Bound | | | | |
| Antibody | (RU ± SE) | (RU) | kₐ (M⁻¹s⁻¹) | k_{d} (s⁻¹) | K_{D} (Molar) | t_{1/2} (min) |
| H4H10423P | 96 ± 1.2 | 4.4 | NB | NB | NB | NB |
| H4H10429P | 142.8 ±1.3 | 25.3 | 3.43E+06 | 3.43E−02 | 9.98E−09 | 0.3 |
| H4H10432P2 | 134.1 ± 1.7 | 5.1 | NB | NB | NB | NB |
| H4H10436P2 | 132 ± 1.4 | 38.1 | 9.78E+06 | 1.36E−03 | 1.39E−10 | 8.5 |
| H4H10440P2 | 94 ± 4.5 | 20.9 | 1.28E+06 | 4.19E−03 | 3.29E−09 | 2.8 |
| H4H10442P2 | 173.1 ± 1.4 | 4.4 | NB | NB | NB | NB |
| H4H10430P | 105.8 ± 1.3 | 3.6 | NB | NB | NB | NB |
| H4H10446P2 | 131.4 ± 1.2 | 3.8 | NB | NB | NB | NB |
| H4H10468P2 | 95.5 ± 1 | 3.4 | NB | NB | NB | NB |
| Control 1 | 90.2 ± 0.9 | 2.7 | NB | NB | NB | NB |

TABLE 7
Binding Characteristics of Anti-Activin A Antibodies to Activin AB at 25° C.

| | Amount of | 50 nM | | | | |
|---|---|---|---|---|---|---|
| | mAb | Ag | | | | |
| | Captured | Bound | | | | |
| Antibody | (RU ± SE) | (RU) | kₐ (M⁻¹s⁻¹) | k_{d} (s⁻¹) | K_{D} (Molar) | t_{1/2} (min) |
| H4H10423P | 81.3 ± 0.5 | 14.7 | 6.13E+05 | 2.03E−02 | 3.31E−08 | 0.6 |
| H4H10429P | 110.7 ± 0.5 | 40.0 | 4.53E+06 | 1.03E−04 | 2.28E−11 | 111.7 |
| H4H10432P2 | 101.2 ± 1.6 | 38.3 | 4.00E+06 | 2.27E−03 | 5.68E−10 | 5.1 |
| H4H10436P2 | 107.5 ± 0.3 | 28.2 | 7.66E+06 | 2.61E−04 | 3.41E−11 | 44.2 |
| H4H10440P2 | 73.7 ± 0.4 | 15.5 | 2.97E+06 | 5.26E−04 | 1.77E−10 | 22.0 |
| H4H10442P2 | 133.3 ± 0.6 | 34.6 | 5.53E+06 | 1.77E−03 | 3.20E−10 | 6.5 |
| H4H10430P | 86.9 ± 0.5 | 33.0 | 1.17E+07 | 2.17E−04 | 1.85E−11 | 53.3 |
| H4H10446P2 | 99.8 ± 0.4 | 31.9 | 4.99E+06 | 4.06E−03 | 8.15E−10 | 2.8 |
| H4H10468P2 | 92.1 ± 0.2 | 34.7 | 3.76E+06 | 2.09E−03 | 5.56E−10 | 5.5 |
| Control 1 | 83.5 ± 0.6 | 31.1 | 3.44E+06 | 2.83E−04 | 8.22E−11 | 40.9 |

TABLE 8
Binding Characteristics of Anti-Activin A Antibodies to Activin AB at 37° C.

| | Amount of | 50 nM | | | | |
|---|---|---|---|---|---|---|
| | mAb | Ag | | | | |
| | Captured | Bound | | | | |
| Antibody | (RU ± SE) | (RU) | kₐ (M⁻¹s⁻¹) | k_{d} (s⁻¹) | K_{D} (Molar) | t_{1/2} (min) |
| H4H10423P | 90.8 ± 1.2 | 21.7 | 8.80E+05 | 2.13E−02 | 2.42E−08 | 0.5 |
| H4H10429P | 137.7 ± 1.2 | 50.0 | 6.47E+06 | 4.88E−04 | 7.55E−11 | 23.6 |
| H4H10432P2 | 127.7 ± 1.3 | 44.4 | 5.40E+06 | 5.92E−03 | 1.10E−09 | 2.0 |
| H4H10436P2 | 126.8 ± 0.8 | 33.9 | 1.03E+07 | 4.58E−04 | 4.43E−11 | 25.2 |
| H4H10440P2 | 88.9 ± 1.7 | 17.7 | 5.20E+06 | 1.63E−03 | 3.14E−10 | 7.1 |
| H4H10442P2 | 166.5 ± 1.7 | 45.9 | 9.17E+06 | 4.25E−03 | 4.64E−10 | 2.7 |
| H4H10430P | 101.6 ± 1.2 | 41.0 | 1.01E+07 | 5.41E−04 | 5.35E−11 | 21.3 |
| H4H10446P2 | 126.6 ± 1.2 | 41.5 | 6.08E+06 | 8.17E−03 | 1.34E−09 | 1.4 |
| H4H10468P2 | 92.2 ± 0.8 | 34.5 | 5.03E+06 | 4.43E−03 | 8.80E−10 | 2.6 |
| Control 1 | 86.4 ± 0.6 | 29.3 | 3.77E+06 | 7.38E−04 | 1.96E−10 | 15.7 |

TABLE 9
Binding Characteristics of Anti-Activin A Antibodies to Activin AC at 25° C.

| | Amount of | 50 nM | | | | |
|---|---|---|---|---|---|---|
| | mAb | Ag | | | | |
| | Captured | Bound | | | | |
| Antibody | (RU ± SE) | (RU) | kₐ (M⁻¹s⁻¹) | k_{d} (s⁻¹) | K_{D} (Molar) | t_{1/2} (min) |
| H4H10423P | 79.9 ± 0.4 | −0.8 | NB | NB | NB | NB |
| H4H10429P | 108.9 ± 0.5 | 28.0 | 9.13E+05 | 9.10E−05 | 9.97E−11 | 126.9 |
| H4H10432P2 | 101.6 ± 0.7 | 34.9 | 6.29E+05 | 1.87E−03 | 2.98E−09 | 6.2 |
| H4H10436P2 | 106.7 ± 0.4 | 30.1 | 6.98E+05 | 1.56E−03 | 2.24E−09 | 7.4 |
| H4H10440P2 | 73.5 ± 0.4 | 11.8 | 5.13E+05 | 2.27E−04 | 4.42E−10 | 50.8 |
| H4H10442P2 | 132.5 ± 3.1 | 18.6 | 1.31E+06 | 2.05E−03 | 1.57E−09 | 5.6 |
| H4H10430P | 85.1 ± 0.3 | 23.6 | 1.23E+06 | 1.09E−02 | 8.86E−09 | 1.1 |
| H4H10446P2 | 96.9 ± 0.5 | 12.6 | 1.04E+06 | 1.22E−02 | 1.18E−08 | 0.9 |
| H4H10468P2 | 91.4 ± 0.3 | 17.2 | 7.98E+05 | 5.92E−03 | 7.41E−09 | 2.0 |
| Control 1 | 82.5 ± 0.3 | 22.3 | 5.58E+05 | 2.25E−03 | 4.03E−09 | 5.1 |

TABLE 10
Binding Characteristics of Anti-Activin A Antibodies to Activin AC at 37° C.

| | Amount of | 50 nM | | | | |
|---|---|---|---|---|---|---|
| | mAb | Ag | | | | |
| | Captured | Bound | | | | |
| Antibody | (RU ± SE) | (RU) | kₐ (M⁻¹s⁻¹) | k_{d} (s⁻¹) | K_{D} (Molar) | t_{1/2} (min) |
| H4H10423P | 85.9 ± 1.1 | 0.0 | NB | NB | NB | NB |
| H4H10429P | 132.6 ± 1.2 | 35.7 | 1.34E+06 | 6.20E−04 | 4.62E−10 | 18.6 |
| H4H10432P2 | 123.8 ± 1.4 | 34.6 | 7.22E+05 | 9.02E−03 | 1.25E−08 | 1.3 |
| H4H10436P2 | 122.9 ± 1.3 | 32.6 | 8.81E+05 | 3.31E−03 | 3.75E−09 | 3.5 |
| H4H10440P2 | 86.6 ± 2.7 | 13.3 | 7.18E+05 | 7.55E−04 | 1.05E−09 | 15.3 |
| H4H10442P2 | 160.1 ± 1.5 | 21.4 | 1.46E+06 | 5.99E−03 | 4.10E−09 | 1.9 |
| H4H10430P | 96.8 ± 1 | 25.3 | 1.20E+06 | 2.00E−02 | 1.67E−08 | 0.6 |
| H4H10446P2 | 120.3 ± 1 | 14.4 | 9.59E+05 | 2.16E−02 | 2.25E−08 | 0.5 |
| H4H10468P2 | 88.4 ± 0.8 | 10.7 | 7.19E+05 | 1.24E−02 | 1.73E−08 | 0.9 |
| Control 1 | 83.2 ± 0.9 | 15.6 | 6.51E+05 | 6.52E−03 | 1.00E−08 | 1.8 |

As shown in Tables 3 and 4, anti-Activin A antibodies of the invention bound to Activin A with K_{D} values ranging from less than 3.18 pM (i.e., ≤3.18E-12) to 745 pM (i.e., 7.45E-10) at 25° C. and with K_{D} values ranging from less than 2.18 pM (i.e., ≤2.18E-12) to 1.77 nM (1.77E-09) at 37° C. As shown in Tables 5 and 6, several of the anti-Activin A antibodies (i.e., H4H10432P2,H4H10442P2, H4H10430P2, H4H10446P2, and H4H10468P2) demonstrated no measurable binding to Activin B at 25° C. or 37° C. Some of the antibodies demonstrated measurable binding to Activin AB with K_{D} values ranging from approximately 18.5 pM (i.e., 1.85E-11) to 33.1 nM (i.e., 3.31E-08) at 25° C. (Table 7) and from approximately 44.3 pM (i.e., 4.43E-11) to 24.2 nM (i.e., 2.42E-08) at 37° C. (Table 8). Some of the antibodies demonstrated measurable binding to Activin AC with K_{D} values ranging from approximately 99.7pM (i.e., 9.97E-11) to 11.8 nM (i.e., 1.18E-08) at 25° C. (Table 9) and from approximately 462 pM (i.e., 4.62E-10) to 22.5 nM (i.e., 2.25E-08) at 37° C. (Table 10). Furthermore, none of the tested anti-Activin A antibodies of the invention demonstrated measurable binding to Inhibin E (data not shown).

### Example 4. Inhibition of Activin A-Mediated Receptor Activation and SMAD Complex Signaling with Anti-Activin A Antibodies

To further characterize anti-Activin A antibodies discussed herein, a bioassay was developed to detect the activation of the activin Type IIA and IIB receptors (ActRIIA and ActRIIB, respectively) and the subsequent phosphorylation and activation of an Activin Type I receptor. The interaction between ActRIIA and ActRIIB and activin leads to the induction of diverse cellular processes including growth regulation, metastatis of cancer cells and differentiation of embryonic stem cells (Tsuchida, K. et al., Cell Commun Signal 7:15 (2009)). Phosphorylation and activation of the Type I receptor leads to phosphorylation of SMAD 2 and 3 proteins which form activated SMAD complexes leading to transcriptional regulation of genes.

To detect the activation of the SMAD complex signal transduction pathway via activin binding to activin Type II receptors, a human A204 rhabdomyosarcoma cell line (ATCC, # HTB-82) was transfected with a Smad 2/3-luciferase reporter plasmid (CAGAx12-Luc; Dennler, 1998) to create the A204/CAGAx12-Luc cell line. A204/CAGAx12-Luc cells were maintained in McCoy's 5A (Irvine Scientific, #9090) supplemented with 10% fetal bovine serum (FBS), penicillin/streptomycin/glutamine and 250 μg/mL of G418. For the bioassay, A204/CAGAx12-Luc cells were seeded onto 96-well assay plates at 10,000 cells/well in low serum media, 0.5% FBS and OPTIMEM (Invitrogen, #31985-070), and incubated at 37° C. and 5% CO₂ overnight. To determine the ligand dose response, Activin A (R&D Systems, #338-AC), Activin B (R&D Systems, #659-AB), Activin AB (R&D Systems, #1066-AB) and Activin AC (R&D Systems, #4879-AC/CF) were serially diluted at 1:3 from 100 to 0.002 nM and added to cells starting along with a control containing no Activin. Activin A, Activin B, Activin AB, and Activin AC were observed to activate the A204/CAGAx12-Luc cell line with EC₅₀ values of 99 pM, 47 pM, 19 pM, and 4.4 nM, respectively. To measure inhibition, antibodies were serially diluted at 1:3 starting from 100 to 0.002 nM, 1000 to 0.02 nM, or 300 to 0.005 nM including control samples containing either an appropriate isotype control antibody or no antibody and added to cells with a constant concentration of 100 pM Activin A, 50 pM Activin B, 30 PM Activin AB or 4 nM Activin AC. Also used as a positive blocking control in this assay was Control 4 (ActRIIB-hFc; SEQ ID No:227). After 5.5 hours of incubation in 37° C. and 5% CO₂, OneGlo substrate (Promega, #E6051) was added and then luciferase activity was detected using a Victor X (Perkin Elmer) instrument. The results were analyzed using nonlinear regression (4-parameter logistics) with Prism 5 software (GraphPad).

As shown in Table 11, anti-Activin A antibodies of the invention blocked 100 pM of Activin A with IC₅₀ values ranging from 39 pM to 3.5 nM, while Control 1 blocked with an IC₅₀ value of 83 pM. A subset of the anti-Activin A antibodies of the invention were tested for blocking Activin B, AB, and AC. Four of the 9 antibodies tested blocked 50 pM of Activin B with IC₅₀ values ranging from 130 pM to 100 nM. Five antibodies of the invention that were tested for Activin B blockade only blocked at high antibody concentrations, while Control 1 did not show any measurable Activin B blockade. Eight antibodies of the invention tested blocked 30 pM of Activin AB with IC₅₀ values ranging from 100 pM to 8.2 nM, while Control 4 blocked with an IC₅₀ value of 540 pM. One antibody, H4H10423P, only demonstrated weak blockade of Activin AB. Seven of the 8 antibodies tested blocked 4 nM of Activin AC with IC₅₀ values ranging from 580 pM to 6.5 nM, while Control 4 blocked with an IC₅₀ value of 1.1 nM. One antibody, H4H10423P, did not demonstrate any blockade of Activin AC. Both mouse IgG (mIgG isotype control) and human IgG (hIgG isotype control) negative controls did not block ligand activation of the receptors.

TABLE 11
Inhibition of Activin A, Activin B, Activin AB, and
Activin AC by anti-Activin A antibodies (IC₅₀ [M])

| | Constant: | | | |
|---|---|---|---|---|
| Antibody | Activin A | Activin B | Activin AB | Activin AC |
| H4H10423P | 2.0E−10 | | Weak | Non- |
| | | | Blocker | Blocker |
| H4H10424P | 7.6E−10 | | | |
| H4H10426P | 2.3E−10 | | | |
| H4H10429P | 1.6E−10 | 7.9E−08 | 2.9E−10 | 5.8E−10 |
| H4H10430P | 6.1E−11 | Block at | 1.0E−10 | 9.3E−10 |
| | | High Conc. | | |
| H4H10432P2 | 1.1E−10 | Block at | 8.0E−10 | 2.8E−09 |
| | | High Conc. | | |
| H4H10433P2 | 1.5E−10 | 1.0E−07 | | |
| H4H10436P2 | 2.0E−10 | 1.3E−10 | 1.4E−10 | 1.3E−09 |
| H4H10437P2 | 2.9E−10 | Block at | | |
| | | High Conc. | | |
| H4H10438P2 | 2.6E−10 | | | |
| H4H10440P2 | 2.8E−10 | 5.2E−09 | 4.3E−10 | 7.5E−10 |
| H4H10442P2 | 5.6E−11 | | 2.2E−09 | 6.5E−09 |
| H4H10445P2 | 5.3E−11 | | | |
| H4H10446P2 | 4.7E−11 | Block at | 8.2E−09 | 5.6E−09 |
| | | High Conc. | | |
| H4H10447P2 | 7.8E−11 | | | |
| H4H10448P2 | 4.6E−11 | | | |
| H4H10452P2 | 5.8E−11 | | | |
| H4H10468P2 | 3.9E−11 | Block at | 2.3E−09 | 3.4E−09 |
| | | High Conc. | | |
| H2aM10965N | 3.5E−09 | | | |
| mIgG isotype | Non-Blocker | | | |
| control | | | | |
| hIgG isotype | Non-Blocker | Non-Blocker | Non-Blocker | Non-Blocker |
| control | | | | |
| Control 1 | 8.3E−11 | Non-Blocker | 5.4E−10 | 1.1E−09 |

The bioassay using A204/CAGAx12-Luc cells could also be stimulated by GDF8 (R&D Systems, Cat #788-G8/CF) and GDF11 (R&D Systems, Cat #1958-GD-010/CF). To test for functional inhibition of these ligands with activin A antibodies, the assay was performed using conditions described above but substituting GDF8 or GDF11 for the activating ligand, which resulted in EC50 values of 188 pM and 84 pM, respectively. In this assay, activation by a constant concentration of 0.50 nM GDF8 or 0.40 nM GDF11 was completely blocked by Control 4 with IC₅₀ values of 298 pM and 214 pM, respectively. Using these same constant concentrations of ligands, no inhibition of either GDF8 or GDF11 was observed by the activin A antibodies, H4H10446P2 and H4H10430P, when tested at up to 100 nM of the antibodies. On a separate day, the activin A antibodies H4H10429P and H4H10436P2 were tested for inhibition in this assay in the presence of constant concentrations of 250 pM GDF8 or 250 pM GDF11, and no inhibition was observed after incubation of the cells with up to 150 nM of the tested activin A antibodies; GDF8 and GDF11 alone in this assay exhibited EC50 values of 124 pM and 166 pM, respectively. These data demonstrate that the Activin A antibodies H4H10446P2, H4H10430P, H4H10429P and H4H10436P2 do not functionally inhibit GDF8 or GDF11.

### Example 5. Blocking of Activin A Binding using Activin A Antibodies

The ability of selected anti-Activin A antibodies to block the interaction of Activin A with its receptors, ActRIIB and ActRIIA, as well as its endogenous antagonist, Follistatin, was determined using a Biacore 3000 instrument. For this experiment, Control 4 (human ActRIIB expressed with a C-terminal human Fc tag (SEQ ID: 227)), human ActRIIA expressed with a C-terminal human Fc tag (hActRIIA-Fc; R&D Systems, #340-R2-100), or Follistatin-288 (R&D Systems, #5836-FS-025) were amine-coupled to a Biacore CM5 sensor surface. Activin A (R&D Systems, #338-AC) at a fixed concentration of 5 nM either alone or mixed with Activin A antibodies, hActRIIA-Fc, hActRIIB-Fc, or isotype control antibody at a final concentration of 60 nM (12-fold molar excess over Activin A) was incubated at room temperature for 1 hour. The antibody-Activin A mixtures were then injected over the amine-coupled Control 4, hActRIIA-Fc, or Follistatin-288 surfaces at a flow rate of 20 uL/min. The binding signal (RU) was measured at 150 seconds after the start of the injection, and this signal was subtracted by the measured RU value for a negative control reference surface to determine the specific binding signal. The percentage of free Activin A binding over the receptor or antagonist surfaces in the presence of each anti-Activin A antibody was calculated as the ratio of the observed specific binding signal divided by the specific binding signal from 5 nM Activin A in the presence of no antibody.

TABLE 12
Blocking of Activin A Binding to Follistatin by anti-Activin A Antibodies

| mAb/protein | | | | | | | |
|---|---|---|---|---|---|---|---|
| concentration | Follistatin-288 surface (3000RU captured)-Normalized to Activin A (% bound RU w/no inhibitor) | | | | | | |
| (nM) | H4H10442P2 | H4H10446P2 | H4H10430P | H4H10440P2 | H4H10429P | H4H10436P2 | H4H10423P |
| 0 | 100 | 100 | 100 | 100 | 100 | 100 | 100 |
| 0.94 | 73 | 77 | 79 | 76 | 97 | 78 | 120 |
| 1.88 | 46 | 54 | 59 | 57 | 80 | 61 | 122 |
| 3.75 | 6 | 7 | 15 | 17 | 20 | 16 | 103 |
| 7.5 | 3 | 3 | 1 | 4 | 1 | 1 | 97 |
| 15 | 3 | 3 | 1 | 2 | 1 | 1 | 96 |
| 30 | 3 | 3 | 1 | 1 | 2 | 2 | 94 |
| 60 | 3 | 3 | 1 | 0 | 3 | 2 | 93 |

| | Follistatin-288 surface (3000RU captured)-Normalized to Activin A (% bound RU w/no inhibitor) | | | | |
|---|---|---|---|---|---|
| | | | | Control | |
| mAb/protein | | | | 4 | isotype |
| concentration | Control | Control | hActRIIA- | (hActRIIB- | (−) |
| (nM) | 1 | 3 | hFc | hFc) | control |
| 0 | 100 | 100 | 100 | 100 | 100 |
| 0.94 | 83 | 172 | 156 | 169 | 100 |
| 1.88 | 68 | 170 | 148 | 163 | 102 |
| 3.75 | 27 | 145 | 138 | 151 | 97 |
| 7.5 | 0 | 33 | 116 | 120 | 102 |
| 15 | 1 | 5 | 60 | 43 | 102 |
| 30 | 1 | 7 | 11 | 1 | 104 |
| 60 | 2 | 9 | 13 | 1 | 103 |

As shown in Table 12, 6 of the 7 anti-Activin A antibodies tested and both Control 1 and Control 3 blocked the binding of Actin A to Follistatin-288. One antibody, H4H10423P, did not prevent binding of Activin A to Follistatin-288. Control 4 and hActRIIA-Fc blocked the binding of Activin A to Follistatin-288 at higher concentrations.

TABLE 13
Blocking of Activin A Binding to hActRIIA-Fc by anti-Activin A Antibodies

| mAb/protein | | | | | | | |
|---|---|---|---|---|---|---|---|
| concentration | hActRIIA-hFc surface (8000RU captured)-Normalized to Activin A (% bound RU w/no inhibitor) | | | | | | |
| (nM) | H4H10442P2 | H4H10446P2 | H4H10430P | H4H10440P2 | H4H10429P | H4H10436P2 | H4H10423P |
| 0.00 | 100 | 100 | 100 | 100 | 100 | 100 | 100 |
| 0.94 | 114 | 111 | 81 | 75 | 87 | 75 | 112 |
| 1.88 | 114 | 115 | 62 | 52 | 66 | 55 | 114 |
| 3.75 | 95 | 85 | 19 | 17 | 19 | 16 | 111 |
| 7.50 | 105 | 94 | 3 | 6 | 1 | 2 | 110 |
| 15 | 113 | 108 | 2 | 4 | 1 | 2 | 112 |
| 30 | 117 | 98 | 2 | 3 | 1 | 2 | 114 |
| 60 | 118 | 118 | 2 | 3 | 1 | 2 | 116 |

| | hActRIIA-hFc surface (8000RU captured)-Normalized to Activin A (% bound RU w/no inhibitor) | | | | |
|---|---|---|---|---|---|
| | | | | Control | |
| mAb/protein | | | | 4 | isotype |
| concentration | Control | Control | hActRIIA- | (hActRIIB- | (−) |
| (nM) | 1 | 3 | hFc | hFc) | control |
| 0.00 | 100 | 100 | 100 | 100 | 100 |
| 0.94 | 82 | 207 | 236 | 276 | 109 |
| 1.88 | 66 | 190 | 222 | 266 | 112 |
| 3.75 | 28 | 139 | 188 | 231 | 110 |
| 7.50 | 100 | 32 | 128 | 160 | 115 |
| 15 | 1 | 1 | 50 | 51 | 116 |
| 30 | 1 | 1 | 5 | 2 | 118 |
| 60 | 2 | 0 | 3 | 1 | 119 |

As shown in Table 13, 4 of the 7 anti-Activin A antibodies tested and both Control 1 and Control 3 blocked the binding of hActRIIA-Fc to Activin A. Three antibodies, H4H10442P2, H4H10446P2, and H4H10423P, did not prevent binding of Activin A to hActRIIA-Fc. Control 4 and hActRIIA-Fc blocked the binding of Activin A to hActRIIA-Fc.

TABLE 14
Blocking of Activin A Binding to hActRIIB-Fc by anti-Activin A Antibodies

| mAb/protein | | | | | | | |
|---|---|---|---|---|---|---|---|
| concentration | hActRIIB-hFc (Control 4) surface (4000RU captured)-Normalized to Activin A (% bound RU w/no inhibitor) | | | | | | |
| (nM) | H4H10442P2 | H4H10446P2 | H4H10430P | H4H10440P2 | H4H10429P | H4H10436P2 | H4H10423P |
| 0.00 | 100 | 100 | 100 | 100 | 100 | 100 | 100 |
| 0.94 | 110 | 107 | 80 | 79 | 87 | 80 | 93 |
| 1.88 | 106 | 105 | 62 | 58 | 67 | 60 | 78 |
| 3.75 | 88 | 76 | 20 | 19 | 19 | 19 | 47 |
| 7.50 | 103 | 95 | 4 | 7 | 2 | 3 | 42 |
| 15 | 115 | 115 | 3 | 4 | 2 | 2 | 42 |
| 30 | 122 | 89 | 3 | 4 | 2 | 3 | 41 |
| 60 | 124 | 129 | 3 | 4 | 3 | 4 | 41 |

| | hActRIIB-hFc (Control 4) surface (4000RU captured)-Normalized to Activin A (% bound RU w/no inhibitor) | | | | |
|---|---|---|---|---|---|
| | | | | Control | |
| mAb/protein | | | | 4 | isotype |
| concentration | Control | Control | hActRIIA- | (hActRIIB- | (−) |
| (nM) | 1 | 3 | hFc | hFc) | control |
| 0.00 | 100 | 100 | 100 | 100 | 100 |
| 0.94 | 85 | 135 | 131 | 149 | 105 |
| 1.88 | 69 | 133 | 129 | 148 | 105 |
| 3.75 | 31 | 120 | 127 | 144 | 104 |
| 7.50 | 2 | 33 | 113 | 130 | 107 |
| 15 | 2 | 2 | 56 | 51 | 110 |
| 30 | 2 | 1 | 5 | 3 | 111 |
| 60 | 3 | 2 | 5 | 2 | 115 |

As shown in Table 14, 4 of the 7 anti-Activin A antibodies tested and both Control 1 and Control 3 blocked the binding of Activin A to hActRIIB-Fc. Two antibodies, H4H10442P2 and H4H10446P2, did not prevent binding of Activin A to hActRIIB-Fc. One antibody, H4H10423P, demonstrated the ability to partially block the binding of Activin A to hActRIIB-Fc at higher concentrations of antibody tested. Both hActRIIB-Fc and hActRIIA-Fc blocked the binding of Activin A to hActRIIB-Fc.

### Example 6. Activin A Induces Upstream Signaling and Activates Cardiac Stress Genes in Human Induced Pluripotent Stem Cell Cardiomyocytes

For culture of human inducible pluripotent stem cell-derived (IPSC) cardiomyocytes, tissue culture vessels were pre-coated with 10 μg/mL fibronectin (Thermo Fisher Scientific, Waltham, MA, USA) for 1 hour at 37° C. Human iPSC-CMs (iCell Cardiomyocytes²; Fujifilm Cellular Dynamics, Madison, WI, USA) were stored, thawed, and plated according to the manufacturer's instructions. Briefly, cells were flash thawed (37° C., 3 minutes) and slowly diluted in plating medium. For gene expression analysis and phosphorylation assays, 5×10⁵ cells were plated per well of a 12-well plate. For impedance, electrophysiology, and calcium flux assays, cells were plated in 96-well plates at a density of 5×10⁴ cells per well. Cells were maintained in a humidified 37° C. incubator with 5% CO₂, with media changed every 48 hours. Cells were maintained in culture until a synchronous, beating monolayer of cells formed (˜10-14 days) prior to initiating each experiment.

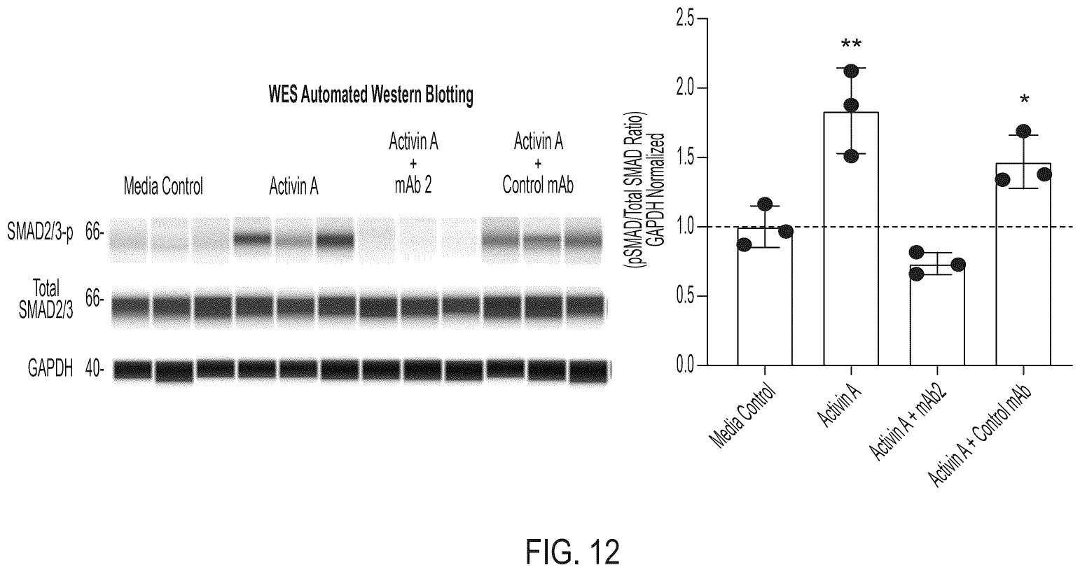

For detection of SMAD phosphorylation, cells were exposed to 1 nM activin A (R&D Systems, Minneapolis, MD, USA) for 30 minutes. Cells were washed twice with cold phosphate buffered saline and lysed using RIPA Lysis and Extraction Buffer (Thermo Fisher Scientific) supplemented with Halt™ Protease and Phosphatase Inhibitor Cocktail (Thermo Fisher Scientific). Lysates were centrifuged (14,000×g, 15 minutes) and total protein quantified using the Pierce BCA Protein Quantitation kit (Thermo Fisher Scientific). Protein detection in cell lysates was performed under reducing conditions using a 12-230 kDa Separation Module for the capillary electrophoresis Wes™ System (ProteinSimple, San Jose, CA, USA), according to the manufacturer's instructions. Protein samples were diluted with the 5× reducing buffer to a final concentration of 0.5 mg/mL, denatured (5 minutes, 95° C.), and placed on ice. Cartridge plates were assembled, spun (1000×g, 5 minutes) and placed into the Wes™ instrument. Primary antibodies were obtained from Cell Signaling Technologies (Danvers, MA, USA). Phospho-SMAD2 (Ser465/467)/SMAD3 (Ser423/425) was diluted 1:50, SMAD2/3 was diluted 1:50, and GAPDH was diluted 1:100. The Anti-Rabbit Detection Module (ProteinSimple) is supplied with antibody diluents, a 1× anti-rabbit secondary antibody, a streptavidin-HRP conjugate, and chemiluminescent detection reagents. Protein detection was analyzed using Compass software (ProteinSimple), which quantified area under curves and height for peak chemiluminescent signals from the proteins of interest. SMAD2/3 phosphorylation was significantly increased by 83% in iPSC-CMs exposed to 1 nM activin A (P<0.001) for 30 minutes. An inhibitory activin A antibody (mAb2; H$H10446P2) blocked this increase in SMAD phosphorylation (see FIG. 12). These data demonstrate that activin A induces signaling in iPSC-CMs.

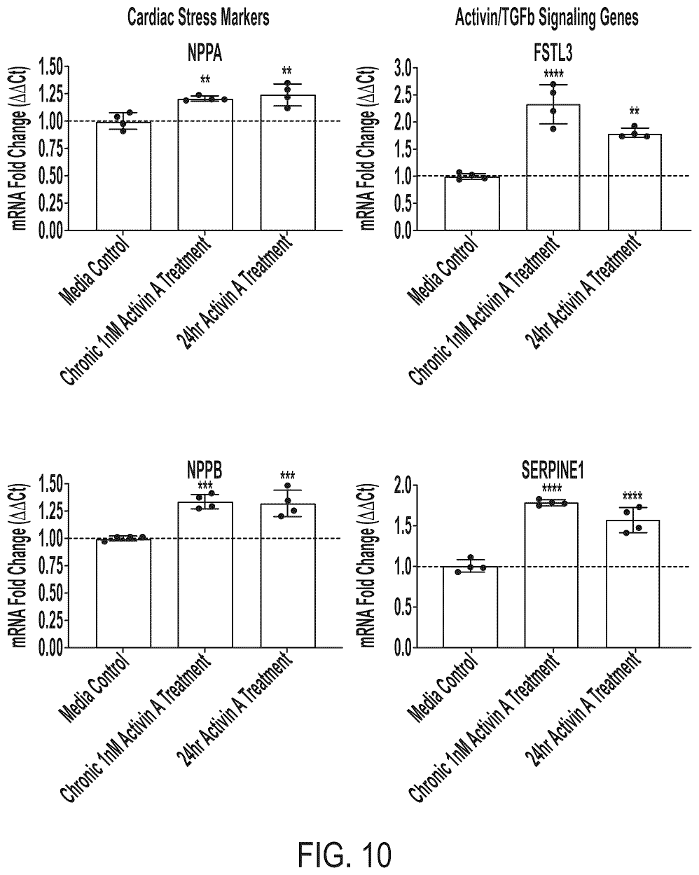

For analysis of gene expression, PCR reactions (20 μL total) contained 10 μL of 2× TaqMan Gene Expression Master Mix (Thermo Fisher Scientific), 1 μL of a 20× TaqMan probe, 5 μL (10 ng) of cDNA, and 4 μL of water and were run on a QuantStudio™ 3 Real-Time PCR System (Thermo Fisher Scientific). Thermocycler settings were as follows: 95° C. for 15 minutes, then 40 cycles of 95° C., 15 seconds followed by 60° C., 60 seconds. Amplification plots were generated by the QuantStudio™ 3 instrument software, and resulting cycle threshold (Ct) values were derived. GAPDH was used as an endogenous control. The delta-delta Ct (2^{-ΔΔCt}) method (Pfaffl 2001) was used to calculate relative fold change in gene expression for all RT-qPCR analyses. Exposure of IPSC cardiomyocytes to Activin A (acutely—24 hours, or chronically—6 treatments) activated expression of downstream Activin A signaling genes FSTL3 (aka FLRG) and Serpine1 (aka PAI-1), as well as atrial natriuretic peptide (NPPA) and B-type natriuretic peptide (NPPB), which are common markers of cardiac stress (see FIG. 10).

### Example 7. Anti-Activin A Induces Contractile and Electrophysiologic Dysfunction of IPSC-Cardiomyocytes that is Blocked by Anti-Activin A Antibodies

Contractility (impedance) and electrophysiology of iPSC-CMs were characterized using the CardioExcyte 96 (Nanion Technologies, Munich, Germany), a hybrid system that can simultaneously record the impedance (contractility) and extracellular field potential (EFP) of a beating monolayer of cardiomyocytes in a label-free environment under physiological culture conditions. In this study, iPSC-CMs were plated on electrode-containing, 96-well plates (NSP-96; Nanion Technologies) and recorded for 30 seconds every 4 hours. Impedance and EFP data were analyzed using Data Control 96 software (Nanion Technologies).

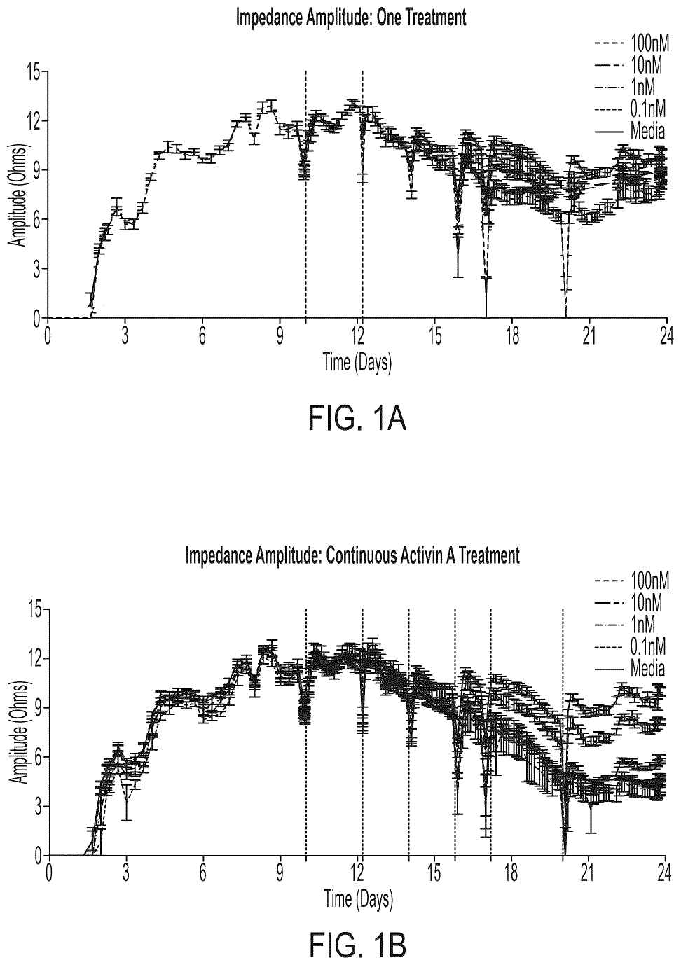

IPSC-cardiomyocytes were plated and contacted with media or varying concentrations of Activin A (R&D Systems) either once or continuously while measuring contractile amplitude (impedance) throughout the experiment. As shown in FIG. 1B, chronic exposure (6 treatments) of the IPSC cardiomyocytes to Activin A resulted in a descending trend in amplitude in a dose dependent manner. Minimal impact of a single exposure was observed (see FIG. 1A).

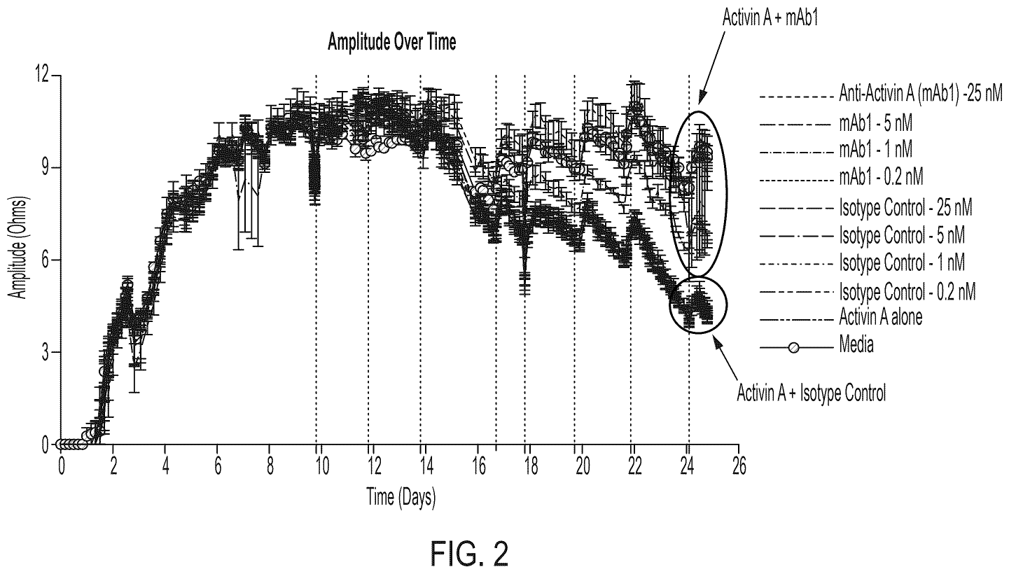

Using plated human IPSC cardiomyocytes, as discussed above, application of an anti-Activin A antibody (mAb1; H4H10430P) prevented the impaired function. As shown in FIG. 2, application of mAb1 prevented the drop in contractile amplitude across all doses tested relative to an isotype control.

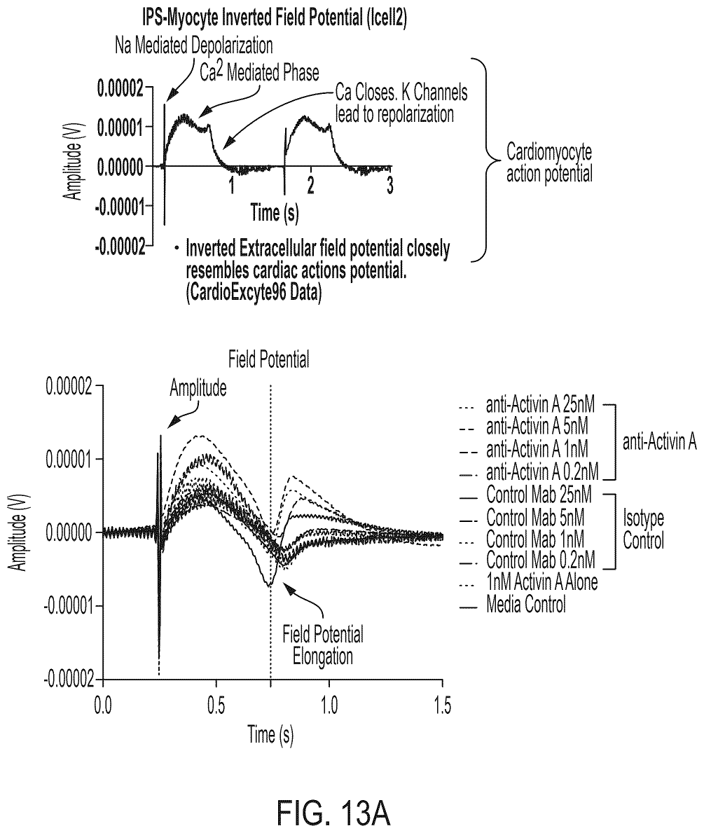

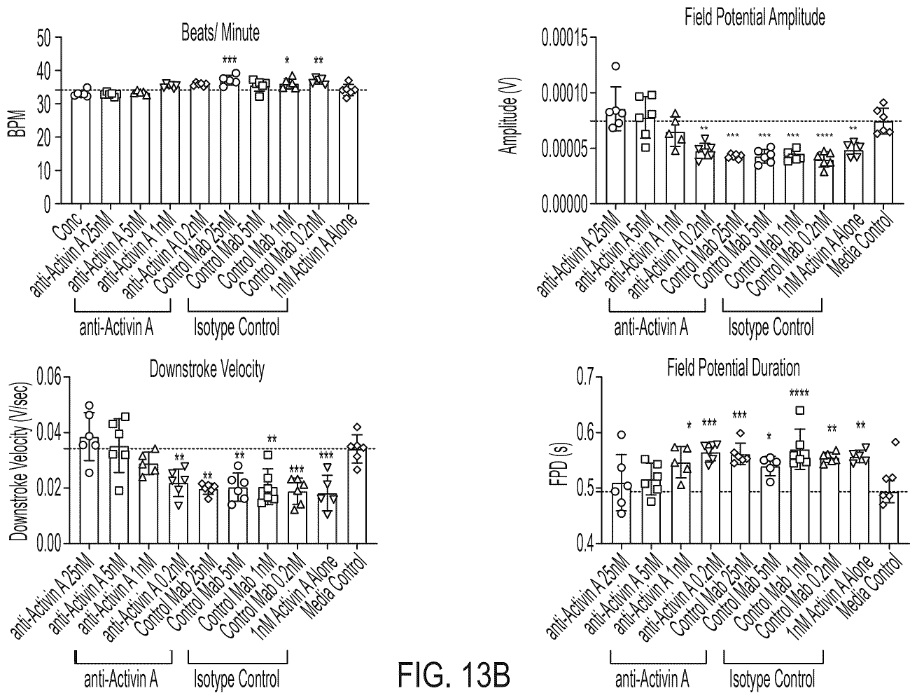

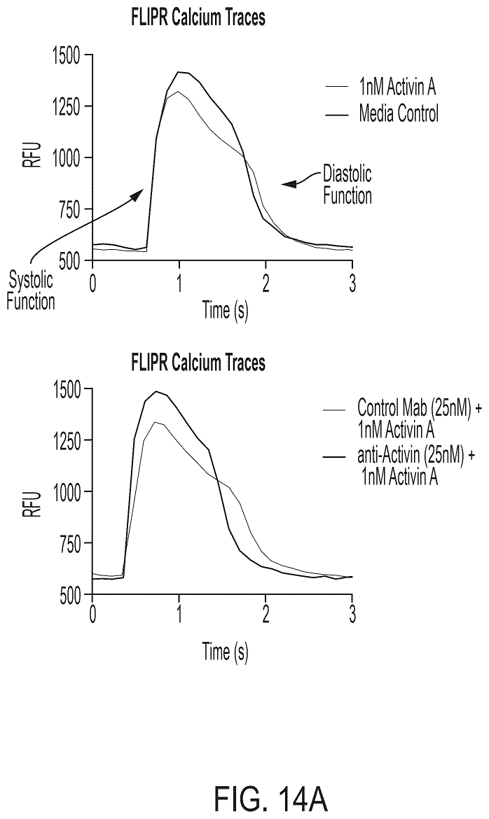

For EFP recording, transient electrical activity outside of the cell is measured, and mean beats were subsequently inversed to resemble the action potential of cardiomyocytes. Amplitude, downstroke velocity (maximal slope during depolarization), and field potential duration (FPDMax: the time between the first deflection for depolarization and the maximum of the repolarization curve) were characterized for each mean beat. An elongated action potential was observed in cardiomyocytes chronically treated with activin A (1 nM) compared to control (0.56±0.01 vs 0.49±0.02 sec, P<0.01). 25 nM of an inhibitory antibody (mAb1) prevented this increase in action potential duration whereas the isotype control antibody did not (0.51±0.05 vs 0.56±0.02 sec). Chronic exposure to activin A caused a reduction in field potential amplitude compared to the media control (48.58±6.52 vs 74.52±11.66 μV, P<0.01). 25 nM of an inhibitory antibody (mAb1) prevented this reduction compared to 25 nM of the isotype control antibody (85.55±19.82 vs 42.87±2.00 μV). Exposure to activin A also reduced the field potential downstroke velocity compared to the media control (0.018±0.006 vs 0.034±0.005 V/sec, P<0.001). Activin A plus 25 nM of the inhibitory antibody (mAb1) prevented this reduction in downstroke velocity where 25 nM of the isotype control antibody did not (0.039±0.009 vs 0.019±0.002 V/sec) (see FIGS. 13A and 13B).

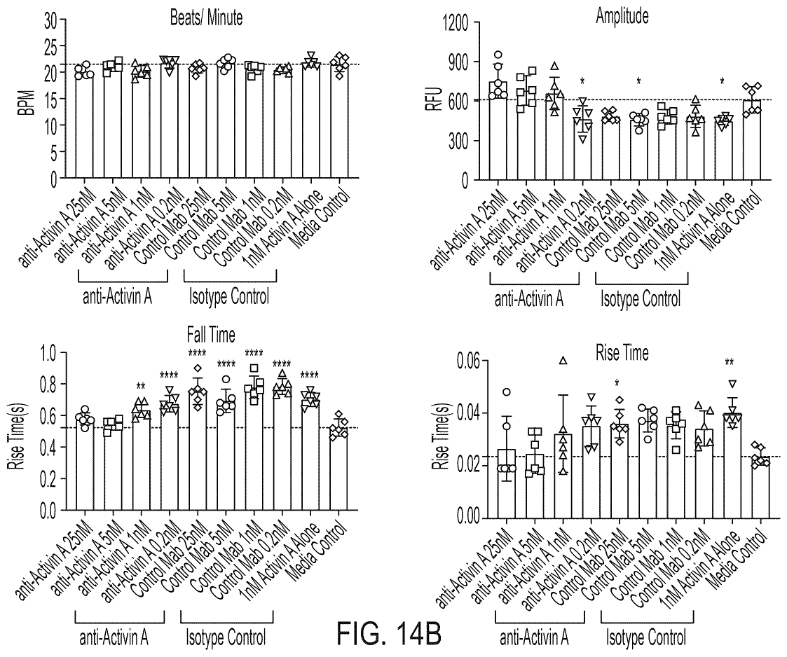

Calcium flux was assessed using an EarlyTox Cardiotoxicity Kit (Molecular Devices, San Jose, CA, USA). Calcium dye loading was performed according to the manufacturer's instructions. EarlyTox calcium dye was resuspended in the supplied buffer and added to the cells in a 1:1 ratio with the cardiomyocyte maintenance media. The plate was incubated for 2 hours (37° C., 5% CO2) before recording calcium flux for 2 minutes at 37° C. on the FLIPR Tetra System (Molecular Devices), using the following parameters: excitation, 470-495 nM; emission, 515-575 nM; exposure time, 50 ms; LED intensity, 50%; interval time 0.1 sec. Calcium flux traces were produced and analyzed using SoftMax Pro Software (Molecular Devices). Chronic treatment of Activin A (1 nM) compared to the media control reduced peak calcium flux amplitude (447±33 vs 609±99 RFU, p<0.05), increased calcium flux falling time (0.70±0.04 sec vs 0.52±0.05 sec, p<0.0001) and increased the calcium flux rising time (0.40±0.06 vs 0.24±0.03 sec, p<0.01). Peak calcium handling amplitude was 759±129 RFU in cells treated with activin A plus inhibitory antibody (mAb1) compared with 484±37 RFU in cells treated with activin A plus isotype control antibody. When compared to the isotype control antibody, an activin A inhibitory antibody (mAb1) prevented the increase in calcium flux falling time (0.58±0.04 vs 0.75±0.08 sec) and rising time (0.27±0.1 vs 0.36±0.05 sec) whereas the isotype control antibody did not (see FIGS. 14A and 14B).

These data demonstrate that elevated levels of Activin A can directly act on cardiomyocytes and may contribute to cardiac dysfunction in heart failure and aged populations.

### Example 8. Serum Levels of Activin A, Follistatin-Related Gene (FLRG) and Plasminogen Activator Inhibitor-1 (PAI-1) are Increased in COVID-19 Patients, and Correlate with Disease Severity

Serum samples were collected from COVID-19 patients, and ELISAs were performed to measure concentrations of Activin A, FLRG and PAI-1 according to standard protocols (R&D Systems). Activin A ELISA samples were diluted 1:1, and FLRG ELISA samples were diluted 1:5. Patient samples were thawed day of, on ice, and each sample was aliquoted for three ELISAs upon thaw to prevent freeze thaw effects. Commercial control serum was used to create plate controls with male and female serum, which were used to normalize data. ELISAs were read on a Bio Tek Synergy Neo2 Multi-Mode Reader. For the FLRG data, if any sample was above the standard curve, the result was entered at the maximum amount of the standard curve (4000 pg/mL).

Descriptive statistics grouped by disease severity and oxygenation requirements were reported as median (interquartile range; IQR) for continuous variables and frequency (percent; %) for categorical variables. Kruskal Wallis tests were used for continuous variables across three or more groups and Wilcoxon signed-rank tests were used for continuous variables between two groups. Fisher's exact tests were used for categorical variables. Kruskal Wallis tests with follow-up Dunn pairwise comparisons (for significant omnibus results) were used to test for differences in Activin A, FLRG, and PAI-1 by disease severity and oxygen requirements at baseline. For longitudinal outcomes, including mortality and clinical score improvement (≥1 point), logistic regression models were used with baseline Activin A, FLRG, and PAI-1 (standardized) as predictors, with and without inclusion of covariates. Fine-Gray subdistribution hazard models were also generated for these longitudinal outcome variables with available time-to-event information. Subjects were split into Viral Load groupings (Low, High) based on a median split of baseline values of each analyte individually. Subdistribution hazard ratios (sHR) for High groups, relative to Low groups, were calculated with and without inclusion of covariates. All-cause mortality and clinical score improvement time-to-event data were censored to Day 60 and Day 29, respectively. Rates of incidence for each outcome during the study were calculated at the censored timepoint.

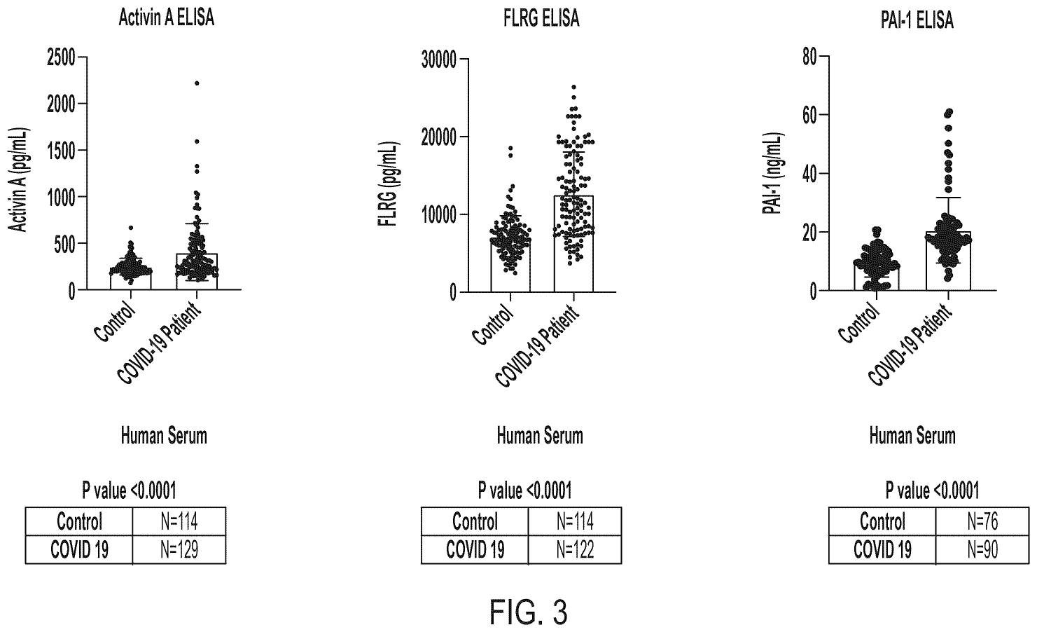

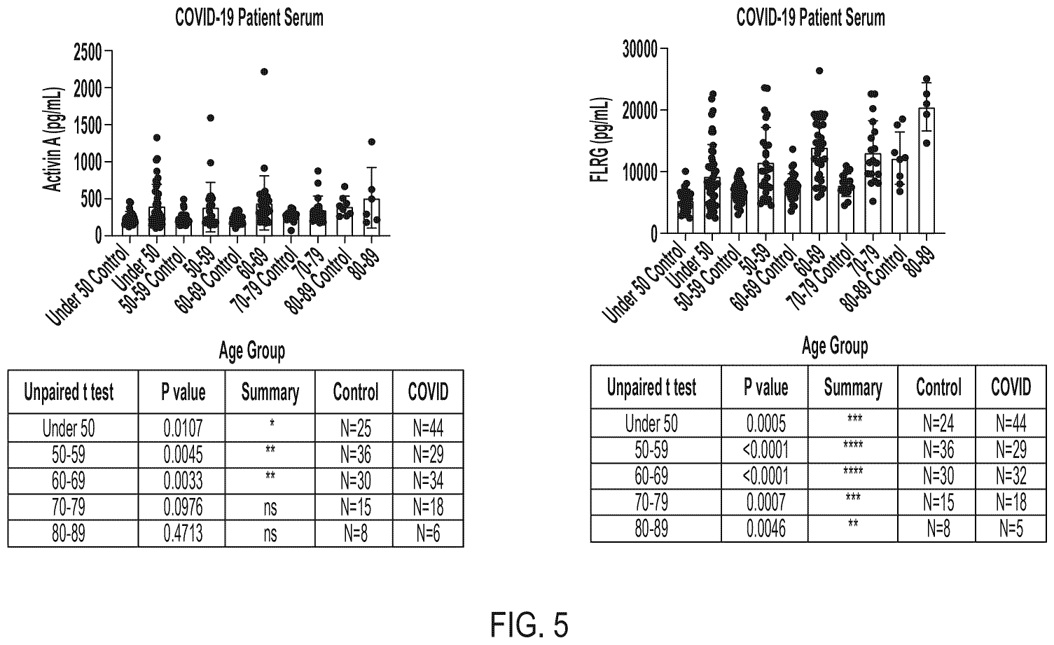

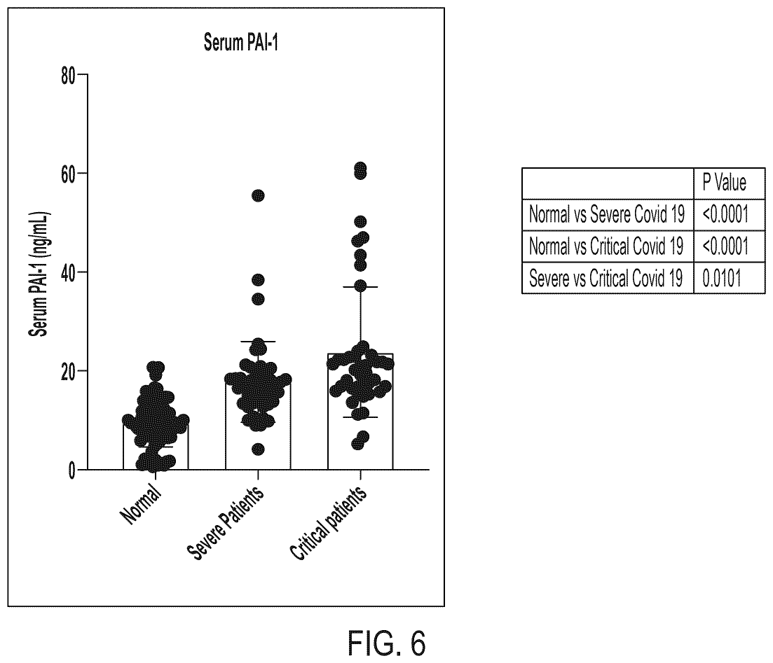

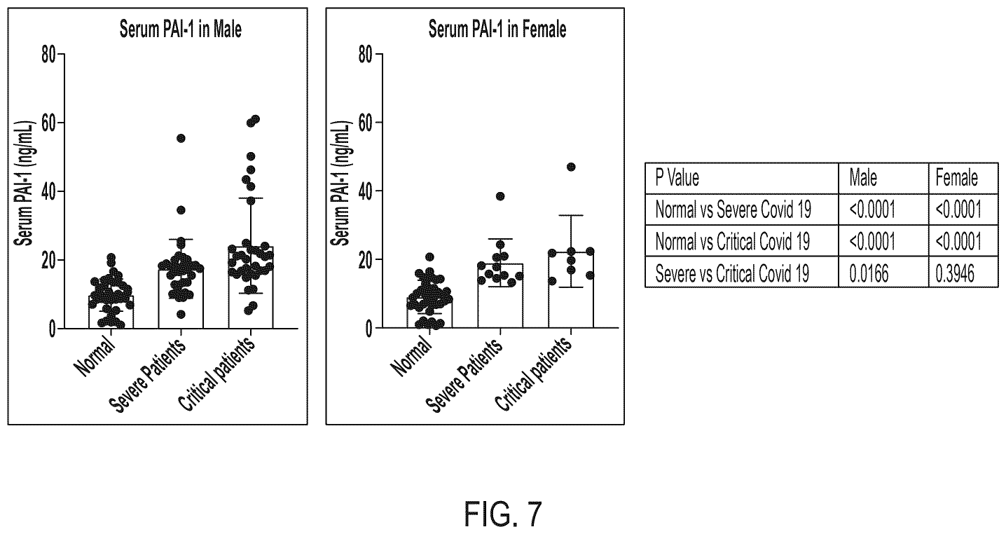

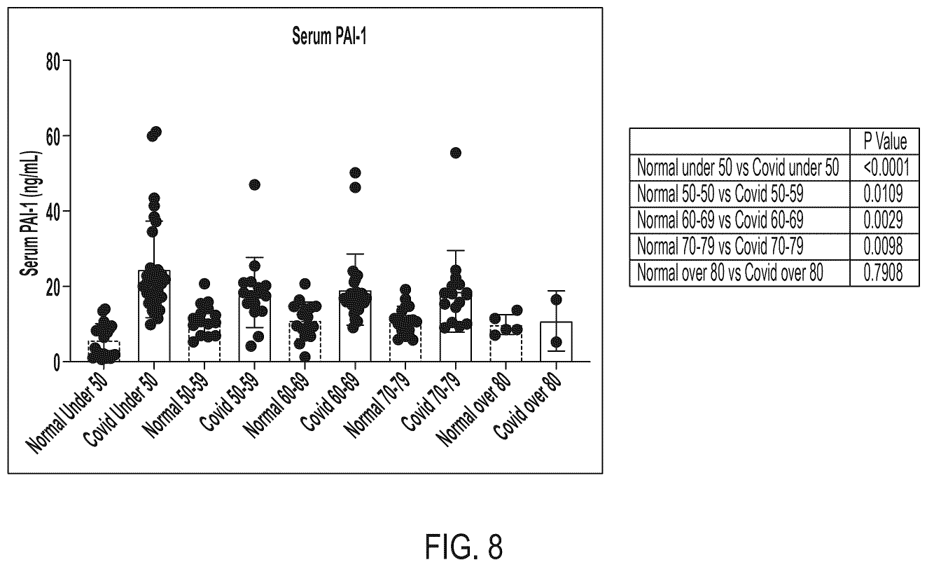

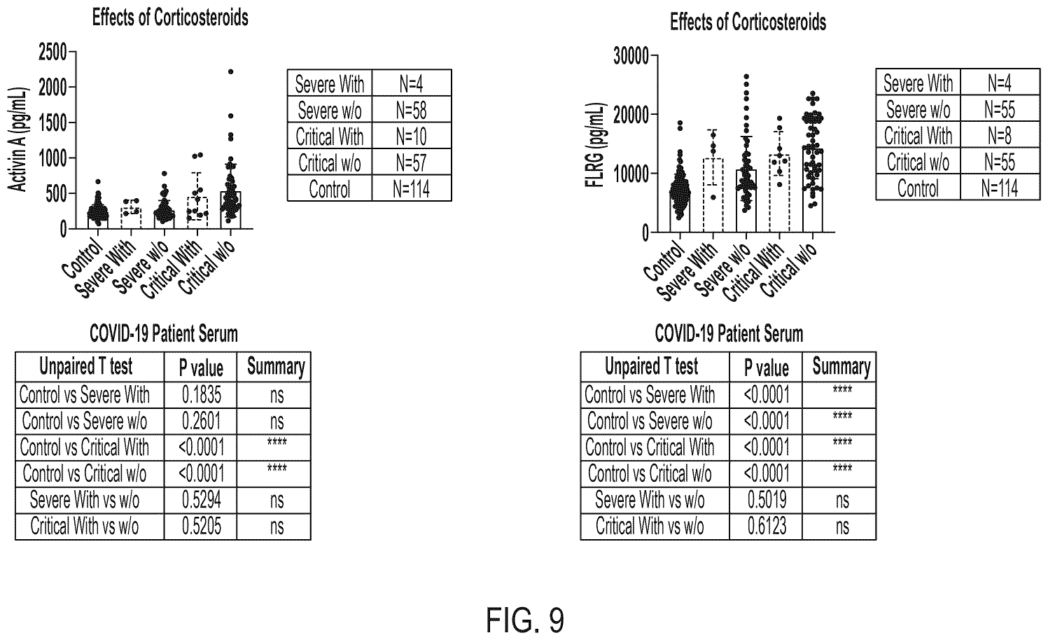

The results are shown in FIGS. 3-9, which demonstrate a significant increase in serum levels of Activin A, FLRG and PAI-1 in COVID-19 patients, relative to controls, and a correlation between serum levels of Activin A, FLRG and PAI-1, and disease severity. Both FLRG and PAI-1 are biomarkers of Activin A pathway activation.

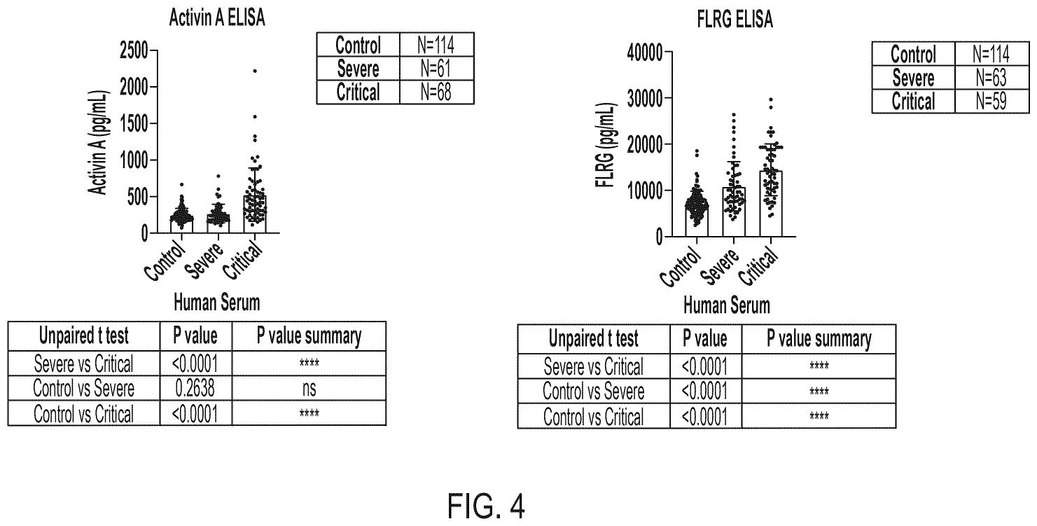

Activin A and FLRG levels were associated with the most severely affected COVID-19 patients, since they were most highly elevated in ICU patients (FIG. 4). The relationship between oxygen requirements at study enrollment and all-cause mortality with baseline of Activin A, FLRG, and PAI-1 in COVID-19 patients was examined. Baseline Activin A was significantly lower in patients who survived (Median=336.8 pg/mL) than in patients who died (Median=547.5 pg/mL; p<0.0001). The same trend was observed for FLRG, which was significantly lower in patients who survived (Median=12141.8 pg/mL) than in patients who died (Median=17633.6 pg/mL; p<0.0001). Baseline PAI-1 levels did not differ significantly by mortality (p=0.52).

Oxygenation status at baseline was stratified into three categories based on oxygen device type: Low Flow, High Flow, and Invasive Mechanical Ventilation (IMV). Differences between these three groups were observed for Activin A (H(2)=48.2; p<0.0001), FLRG (H(2)=37.1; p<0.0001), and PAI-1 (H(2)=11.3; p=0.0004). Activin A was lowest for patients on Low Flow (Median=236.5 pg/mL), higher for patients on High Flow (403.7 pg/mL), and highest for patients on IMV (Median=499.6 pg/mL). However, Activin A did not significantly differ between patients on High Flow and IMV (z=1.8, p>0.0167). The same trend was observed for FLRG, which was lowest for patients on Low Flow (Median=9399.0 pg/mL), higher for patients on High Flow (Median=14117.1 pg/mL), and highest for patients on IMV (Median=15424.3 pg/mL). All pairwise comparisons were significant (p<0.0167). PAI-1 was also lower in the patients on Low Flow (Median=16.7 ng/mL) compared to patients on High Flow (Median=17.5 ng/ml) and IMV (Median=19.2 ng/ml). However, PAI-1 did not significantly differ between patients on Low Flow and High Flow (z=2.1, p>0.0167) in addition to High Flow and IMV (z=1.6, p>0.0167). These data indicate that Activin A and its pathway marker, FLRG, correlate with need for higher oxygen levels, but PAI-1 levels, which are nonetheless high in all COVID-19 groups, do not predict the need for greater oxygen. A summary of laboratory results, study progression and clinical outcomes for patients grouped by baseline supplemental oxygen requirements is shown in Table 15, below.

TABLE 15
Activin pathway laboratory results, study progression, and clinical
outcomes grouped by baseline supplemental oxygen requirements.

| | | | |
|---|---|---|---|
| | | Invasive | |
| | | Mechanical | |
| Low Flow | High Flow | Ventilation | |
| (n = 62) | (n = 134) | (n = 116) | p |

Activin Pathway

| | | | |
|---|---|---|---|
| Activin A | 236.5 | 403.7 | 499.6 |
| (pg/mL) | (182.2-346.1) | (271.7-607.5) | (324.9-726.9) |
| FLRG | 9399 | 14117 | 15424 |
| (pg/mL) | (7233-12497) | (9889-18800) | (11239-22936) |
| PAI-1 | 16.7 | 17.5 | 19.2 |
| (ng/mL) | (12.7-18.6) | (13.7-22.5) | (15.5-25.6) |

Study Progression (days)

| | | | |
|---|---|---|---|
| Fever | 1 | 2 | 4 |
| | (1-3) | (1-4) | (1-7) |
| Resp. | 2 | 4 | 6 |
| rate >24 bpm | (1-6) | (1-11) | (3-13) |
| Hypoxemia | 8 | 12 | 17 |
| | (5-10) | (8-24) | (10-28) |
| Supplemental | 7 | 12 | 17 |
| oxygen use | (4-10) | (8-23) | (10-28) |

Clinical Outcomes

| | | | |
|---|---|---|---|
| All-cause | 8 | 33 | 45 |
| mortality | (12.9) | (24.6) | (38.8) |
| Clinical | 51 | 88 | 58 |
| improvement | (82.2) | (65.7) | (50.0) |
| (1 pt) | | | |
| Improvement | 44 | 92 | 65 |
| in oxygenation | (71.0) | (68.7) | (56.0) |
| Discharge | 50 | 83 | 46 |
| | (80.6) | (61.9) | (39.7) |

Note:
Summary statistics are presented as median (IQR) for continuous variables and count (%) for categorical variables.

As shown in Table 15, tor COVID-19 patients, ActivinA and HLKG (but not other pathway markers, such as PAI-1) are predictive of the worst outcomes of COVID-19, including the need for more invasive oxygenation, the time necessary for hospital treatment, and the likelihood of mortality.

Given the unique correlation of ActivinA and its pathway marker FLRG to the need for oxygenation, and the risk of death, in COVID-19 patients, the mechanism by which inflammatory cytokines induce Activin A was investigated.

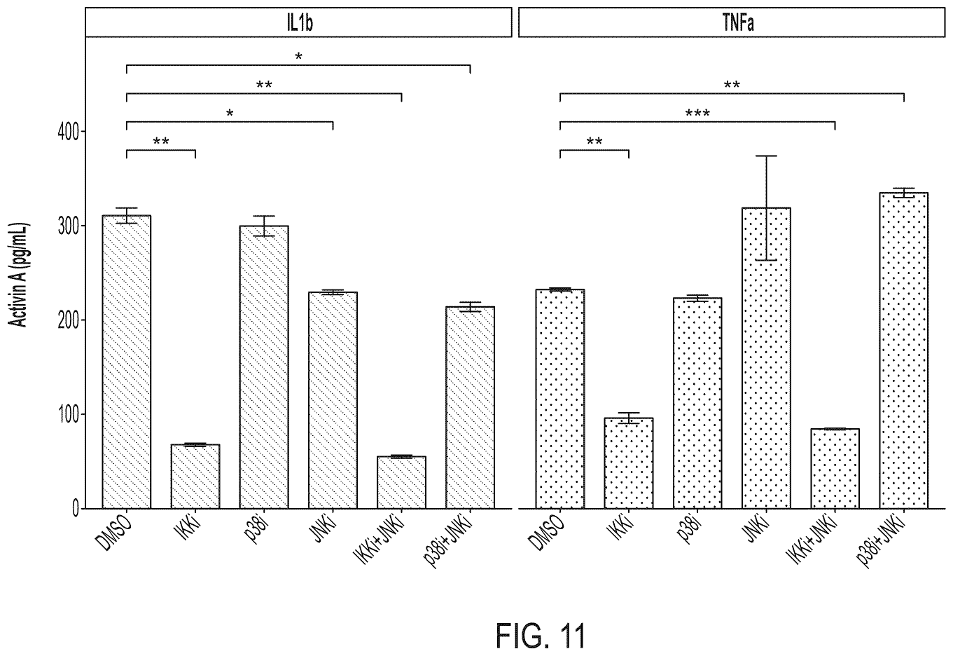

Cook Myosite Human Skeletal Muscle derived cells (SKMDC) were differentiated for 5 days, and co-treated with 100 ng/ml IL1b or TNFa and Activin A induction was followed, with each of the following additional treatments: DMSO (as a negative control, containing vehicle alone), IKKi (an inhibitor of I Kappa Kinase, which is downstream of cytokine stimulation; 3 uM Withaferin A), p38i (an inhibitor of p38; 0.3 uM SB203580), JNKi (an inhibitor of Jnk; 30 uM SP600125), a combination of IKKi+JNKi, or a combination of p38i+JNKi for 24 hours. Activin A concentration in conditioned media were quantified by ELISA. Within each main treatment condition (IL1b, TNFa), Activin A induction for each cotreatment was compared to DMSO cotreatment using pairwise t-tests. Results are shown in FIG. 11. Only significant (p<0.05) Bonferroni corrected comparisons are shown. Within a group, each point is a technical replicate. Relative to DMSO treatment, Activin A induction after either IL-1 or TNFalpha treatment was significantly lower in IKKi treated cells (t(2)=41.4, p=0.0006). In contrast, cells treated with either the JNKi. and p38i+JNKi conditions exhibited much less inhibition of Activin A induction, relative to DMSO treatment. Combination treatment using IKKi resulted in a similar inhibition as treatment with IKKi alone. Thus, IL1 and TNF induced Activin A via the IKK/NF-kappaB pathway, independent of p38 or Jnk, and increased cytokine levels (e.g., IL-1 and TNFα) are associated with an increased need for supplemental oxygen and risk of death in COVID-19 patients.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

TABLE 16
Sequences Excluded from ST.26-Formatted
Sequence Listing

| SEQ ID NO: | Sequence |
|---|---|
| 13 | tgggcatct |
| 14 | WAS |
| 29 | gctgcatcc |
| 30 | AAS |
| 45 | Gagacatcc |
| 46 | ETS |
| 61 | Gatgcttcc |
| 62 | DAS |
| 77 | Gatgcatcc |
| 78 | DAS |
| 93 | Gctgcatcc |
| 94 | AAS |
| 149 | Ggggcaagt |
| 150 | GAS |
| 213 | Ggtgcatcc |
| 214 | GAS |
| 223 | TTS |

What is claimed is:

1. A method of preventing or treating cardiac dysfunction or heart failure in a subject in need thereof, the method comprising administering an Activin A specific antagonist to the subject.

2. The method of claim 1, wherein the Activin A specific antagonist is an anti-Activin A antibody or antigen-binding fragment thereof.

3. The method of claim 2, wherein the antibody or antigen-binding fragment thereof specifically binds Activin A with a binding dissociation equilibrium constant (K_{D}) of less than about 5 pM as measured in a surface plasmon resonance assay at 25° C.

4. The method of claim 2, wherein the antibody or antigen-binding fragment thereof specifically binds Activin A with a K_{D} of less than about 4 pM as measured in a surface plasmon resonance assay at 25° C.

5. The method of claim 2, wherein the antibody or antigen-binding fragment thereof specifically binds Activin A with a binding association equilibrium constant (Kₐ) of less than about 500 nM.

6. The method of any one of claims 1-5, wherein the antibody or antigen-binding fragment thereof blocks binding of at least one Activin A receptor to Activin A.

7. The method of any one of claims 1-5, wherein the antibody or antigen-binding fragment thereof blocks activation of at least one Activin A receptor by Activin A.

8. The method of claim 7, wherein the antibody or antigen-binding fragment thereof does not significantly block binding of Activin A to an Activin Type II receptor.

9. The method of claim 6, wherein the antibody or antigen-binding fragment thereof blocks Activin A binding to an Activin A receptor with an IC₅₀ value of less than about 80 pM as measured in an in vivo receptor/ligand binding bioassay at 25° C.

10. The method of claim 9, wherein the antibody or antigen-binding fragment thereof blocks Activin A binding to an Activin A receptor with an IC₅₀ value of less than about 60 pM as measured in an in vivo receptor/ligand binding bioassay at 25° C.

11. The method of claim 2, wherein the antibody or antigen-binding fragment thereof inhibits binding of Activin A to an Activin A receptor selected from the group consisting of Activin Type IIA receptor (ActRIIA), Activin Type IIB receptor (ActRIIB), and Activin Type I receptor.

12. The method of claim 2, wherein the antibody or antigen-binding fragment thereof inhibits Activin A-mediated activation of SMAD complex signaling.

13. The method of any one of claims 1-12, wherein the antibody or antigen-binding fragment comprises: (a) the complementarity determining regions (CDRs) of a heavy chain variable region (HCVR) comprising an amino acid sequence selected from the group consisting of SEQ ID NOs: 2, 18, 34, 50, 66, 82, 98, 106, 114, 122, 130, 138, 154, 162, 170, 178, 186, 194, and 202; and (b) the CDRs of a light chain variable region (LCVR) comprising an amino acid sequence selected from the group consisting of SEQ ID NOs: 10, 26, 42, 58, 74, 90, 146, and 210.

14. The method of claim 13, wherein the antibody or antigen-binding fragment comprises the heavy and light chain CDRs of a HCVR/LCVR amino acid sequence pair selected from the group consisting of: SEQ ID NOs: 2/10, 18/26, 34/42, 50/58, 66/74, 82/90, 98/90, 106/90, 114/90, 122/90, 130/90, 138/146, 154/146, 162/146, 170/146, 178/146, 186/146, 194/146, and 202/210.

15. The method of claim 14, wherein the antibody or antigen-binding fragment thereof comprises HCDR1-HCDR2-HCDR3-LCDR1-LCDR2-LCDR3 domains comprising the amino acid sequences, respectively, selected from the group consisting of: SEQ ID NOs: 4-6-8-12-14-16; 20-22-24-28-30-32; 36-38-40-44-46-48; 52-54-56-60-62-64; 68-70-72-76-78-80; 84-86-88-92-94-96; 100-102-104-92-94-96; 108-110-112-92-94-96; 116-118-120-92-94-96; 124-126-128-92-94-96; 132-134-136-92-94-96; 140-142-144-148-150-152; 156-158-160-148-150-152; 164-166-168-148-150-152; 172-174-176-148-150-152; 180-182-184-148-150-152; 188-190-192-148-150-152; 196-198-200-148-150-152; and 204-206-208-212-214-216.

16. The method of any one of claims 1-15, wherein the antibody or antigen-binding fragment comprises: (a) a HCVR comprising an amino acid sequence selected from the group consisting of SEQ ID NOs: 2, 18, 34, 50, 66, 82, 98, 106, 114, 122, 130, 138, 154, 162, 170, 178, 186, 194, and 202; and (b) a LCVR comprising an amino acid sequence selected from the group consisting of SEQ ID NOs: 10, 26, 42, 58, 74, 90, 146, and 210.

17. The method of claim 16, wherein the antibody or antigen-binding fragment comprises a HCVR/LCVR amino acid sequence pair selected from the group consisting of: SEQ ID NOs: 2/10, 18/26, 34/42, 50/58, 66/74, 82/90, 98/90, 106/90, 114/90, 122/90, 130/90, 138/146, 154/146, 162/146, 170/146, 178/146, 186/146, 194/146, and 202/210.

18. A method of preventing or treating cardiac dysfunction or heart failure in a subject in need thereof, the method comprising administering an antibody that specifically binds Activin A or an antigen-binding fragment thereof, wherein the antibody or antigen-binding fragment thereof comprises HCDR1-HCDR2-HCDR3-LCDR1-LCDR2-LCDR3 domains comprising the amino acid sequences, respectively, of SEQ ID NOs: 68-70-72-76-78-80.

19. The method of claim 18, wherein the antibody or antigen-binding fragment comprises a HCVR comprising the amino acid sequence of SEQ ID NO: 66, and a LCVR comprising the amino acid sequence of SEQ ID NO: 74.

20. A method of preventing or treating cardiac dysfunction or heart failure in a subject in need thereof, the method comprising administering an antibody that specifically binds Activin A or an antigen-binding fragment thereof, wherein the antibody or antigen-binding fragment thereof comprises HCDR1-HCDR2-HCDR3-LCDR1-LCDR2-LCDR3 domains comprising the amino acid sequences, respectively, of SEQ ID NOs: 164-166-168-148-150-152.

21. The method of claim 18, wherein the antibody or antigen-binding fragment comprises a HCVR comprising the amino acid sequence of SEQ ID NO: 162, and a LCVR comprising the amino acid sequence of SEQ ID NO: 146.

22. The method of any one of claims 1-21, wherein the antibody or antigen-binding fragment is a human antibody comprising an IgG heavy chain constant region.

23. The method of claim 22, wherein the IgG heavy chain constant region is of IgG1 isotype.

24. The method of claim 22, wherein the IgG heavy chain constant region is of IgG4 isotype.

25. The method of any one of claims 1-24, wherein the antibody or antigen-binding fragment is administered in combination with a GDF8 antagonist.

26. The method of claim 25, wherein GDF8 antagonist is selected from the group consisting of a GDF8-inhibiting fusion protein, an anti-GDF8 antibody, and an antigen-binding fragment of an anti-GDF8 antibody.

27. The method of claim 26, wherein the GDF8 antagonist is an anti-GDF8 antibody or antigen-binding fragment thereof.

28. The method of claim 27, wherein the anti-GDF8 antibody or antigen-binding fragment thereof comprises the CDRs of a HCVR comprising the amino acid sequence of SEQ ID NO:217, and the CDRs of a LCVR comprising the amino acid sequences of SEQ ID NO:221.

29. The method of claim 28, wherein the anti-GDF8 antibody or antigen-binding fragment thereof comprises HCDR1-HCDR2-HCDR3-LCDR1-LCDR2-LCDR3 domains comprising the amino acid sequences, respectively, of SEQ ID NOs: 218-219-220-222-223-224.

30. The method of claim 29, wherein the anti-GDF8 antibody or antigen-binding fragment thereof comprises a HCVR comprising the amino acid sequence of SEQ ID NO: 217, and a LCVR comprising the amino acid sequence of SEQ ID NO: 221.

31. The method of any one of claims 1-30, wherein the subject has been diagnosed with a viral infection.

32. The method of claim 31, wherein the viral infection is a coronavirus infection.

33. The method of claim 32, wherein the coronavirus is severe acute respiratory syndrome coronavirus 2 (SARS-COV-2).

34. The method of claim 33, wherein the subject has severe COVID-19 symptoms.

35. The method of claim 33, wherein the subject has critical COVID-19 symptoms.

36. A method of treating COVID-19 in a subject that has tested positive for a severe acute respiratory syndrome coronavirus 2 (SARS-COV-2) infection, the method comprising administering an Activin A specific antagonist to the subject.

37. The method of claim 36, wherein the Activin A specific antagonist is an anti-Activin A antibody or antigen-binding fragment thereof.

38. The method of claim 37, wherein the antibody or antigen-binding fragment comprises the heavy and light chain CDRs of a HCVR/LCVR amino acid sequence pair selected from the group consisting of: SEQ ID NOs: 2/10, 18/26, 34/42, 50/58, 66/74, 82/90, 98/90, 106/90, 114/90, 122/90, 130/90, 138/146, 154/146, 162/146, 170/146, 178/146, 186/146, 194/146, and 202/210.

39. The method of claim 38, wherein the antibody or antigen-binding fragment thereof comprises HCDR1-HCDR2-HCDR3-LCDR1-LCDR2-LCDR3 domains comprising the amino acid sequences, respectively, selected from the group consisting of: SEQ ID NOs: 4-6-8-12-14-16; 20-22-24-28-30-32; 36-38-40-44-46-48; 52-54-56-60-62-64; 68-70-72-76-78-80; 84-86-88-92-94-96; 100-102-104-92-94-96; 108-110-112-92-94-96; 116-118-120-92-94-96; 124-126-128-92-94-96; 132-134-136-92-94-96; 140-142-144-148-150-152; 156-158-160-148-150-152; 164-166-168-148-150-152; 172-174-176-148-150-152; 180-182-184-148-150-152; 188-190-192-148-150-152; 196-198-200-148-150-152; and 204-206-208-212-214-216.

40. The method of any one of claims 37-39, wherein the antibody or antigen-binding fragment comprises: (a) a HCVR comprising an amino acid sequence selected from the group consisting of SEQ ID NOs: 2, 18, 34, 50, 66, 82, 98, 106, 114, 122, 130, 138, 154, 162, 170, 178, 186, 194, and 202; and (b) a LCVR comprising an amino acid sequence selected from the group consisting of SEQ ID NOs: 10, 26, 42, 58, 74, 90, 146, and 210.

41. The method of claim 40, wherein the antibody or antigen-binding fragment comprises a HCVR/LCVR amino acid sequence pair selected from the group consisting of: SEQ ID NOs: 2/10, 18/26, 34/42, 50/58, 66/74, 82/90, 98/90, 106/90, 114/90, 122/90, 130/90, 138/146, 154/146, 162/146, 170/146, 178/146, 186/146, 194/146, and 202/210.

42. The method of claim 37, wherein the antibody or antigen-binding fragment thereof comprises HCDR1-HCDR2-HCDR3-LCDR1-LCDR2-LCDR3 domains comprising the amino acid sequences, respectively, of SEQ ID NOs: 68-70-72-76-78-80.

43. The method of claim 42, wherein the antibody or antigen-binding fragment comprises a HCVR comprising the amino acid sequence of SEQ ID NO: 66, and a LCVR comprising the amino acid sequence of SEQ ID NO: 74.

44. The method of claim 37, wherein the antibody or antigen-binding fragment thereof comprises HCDR1-HCDR2-HCDR3-LCDR1-LCDR2-LCDR3 domains comprising the amino acid sequences, respectively, of SEQ ID NOs: 164-166-168-148-150-152.

45. The method of claim 44, wherein the antibody or antigen-binding fragment comprises a HCVR comprising the amino acid sequence of SEQ ID NO: 162, and a LCVR comprising the amino acid sequence of SEQ ID NO: 146.

46. The method of any one of claims 37-45, wherein the antibody or antigen-binding fragment is a human antibody comprising an IgG heavy chain constant region.

47. The method of claim 46, wherein the IgG heavy chain constant region is of IgG1 isotype.

48. The method of claim 46, wherein the IgG heavy chain constant region is of IgG4 isotype.

49. The method of any one of claims 36-48. wherein the subject has severe COVID-19 symptoms requiring supplemental oxygen administration.

50. The method of any one of claims 36-48, wherein the subject has critical COVID-19 symptoms requiring mechanical ventilation or treatment in an intensive care unit.
